(19) United States
(12) Patent Application Publication
(10) Pub. No.: US 2023/0242672 A1
(43) Pub. Date: Aug. 3, 2023

(54) ACE2-FC FUSION PROTEINS AND METHODS OF USE

(71) Applicant: Gliknik Inc., Baltimore, MD (US)

(72) Inventors: David S. BLOCK, Baltimore, MD (US); Henrik OLSEN, Baltimore, MD (US)

(21) Appl. No.: 18/002,935

(22) PCT Filed: Jun. 25, 2021
(86) PCT No.: PCT/US2021/039131

§ 371 Date: Dec. 22, 2022

Related U.S. Application Data
(60) Provisional application No. 63/176,959, filed on Apr. 20, 2021.
(60) Provisional application No. 63/175,851, filed on Apr. 16, 2021.
(60) Provisional application No. 63/153,592, filed on Feb. 25, 2021.
(60) Provisional application No. 63/068,534, filed on Aug. 21, 2020.
(60) Provisional application No. 63/054,127, filed on Jul. 20, 2020.
(60) Provisional application No. 63/044,281, filed on Jun. 25, 2020.

(51) Int. Cl. C07K 16/40; C07K 16/28; C12N 9/48; C12N 15/63; A61P 31/14

(52) U.S. Cl. CPC C07K 16/40; C07K 16/283; C12Y 304/17023; C12N 9/48; C12N 15/63; A61P 31/14; C07K 2317/52; C07K 2317/92; C07K 2319/30

(57) ABSTRACT

The present disclosure provides ACE2-Fc fusion proteins comprising an ACE2 extracellular domain or fragment thereof and one or more Fc domains and methods of use thereof.

### CROSS-REFERENCE TO RELATED APPLICATIONS

This application claims priority to U.S. Provisional No. 63/044,281, filed Jun. 25, 2020; U.S. Provisional No. 63/054,127, filed Jul. 20, 2020; U.S. Provisional No. 63/068,534, filed Aug. 21, 2020; U.S. Provisional No. 63/153,592, filed Feb. 25, 2021; U.S. Provisional No. 63/175,851, filed Apr. 16, 2021; and U.S. Provisional No. 63/176,959, filed Apr. 20, 2021, each of which is incorporated herein by reference in its entirety.

### TECHNICAL FIELD

The present disclosure generally relates to angiotensin-converting enzyme 2 (ACE2) fusion proteins and methods of use thereof. Specifically, the present disclosure provides ACE2-Fc fusion proteins comprising an ACE2 extracellular domain and one or more Fc domains and methods of use thereof.

### SEQUENCE LISTING

The sequence listing associated with this application is provided in text format in lieu of a paper copy and is hereby incorporated by reference into the specification. The name of the text file containing the sequence listing is GLIK-022_06WO_ST25.txt. The text file is 263 kb, was created on Jun. 23, 2021 and is being submitted electronically via EFS-Web.

### BACKGROUND

ACE2 is a critical regulator of the body's balance between pro- and anti-inflammatory states, including through modulation of the RAAS and bradykinin pathways. Certain viruses, such as severe acute respiratory syndrome coronavirus 1 (SARS-CoV-1) and SARS-CoV-2 are responsible for the SARS epidemic in 2002 to 2004 and for the more recent coronavirus disease 2019 (COVID-19) pandemic. SARS-CoV-1 and SARS-CoV-2 bind and gain entry into host cells via several receptors with the primary receptor being the angiotensin-converting enzyme 2 (ACE2) receptor, a type I transmembrane protein highly expressed in the lungs, heart, kidneys and gastrointestinal tract of humans. The interaction between SARS viruses and the ACE2 receptor has been proposed as a potential factor in both infectivity and inflammation, influencing the rate of viral replication and disease severity. Other disease states are characterized by decreased ACE2 or increased Ang II and can be effectively treated with delivery to peripheral tissues of long-acting, enzymatically active ACE2.

### SUMMARY

Although ACE2 is bound and destroyed by some coronaviruses, it primarily functions as a cell surface carboxypeptidase enzyme that cleaves a number of mammalian substrates including components of the renin-angiotensin-aldosterone system (RAAS), angiotensin I (Ang I) and angiotensin II (Ang II), as well as apelin, pro-dynorphin, des-arg⁹-bradykinin, and others. ACE2 plays a central role in RAAS by functioning as a counter-regulator of the ACE-Ang II-AT1 receptor axis, activation of which mediates vasoconstriction, inflammation, and fibrosis. ACE2-mediated cleavage of Ang II generates Ang-(1-7), which binds and activates the G-protein coupled receptor Mas (Chung et al., EbioMedicine 58 (2020) 102907). ACE2-Ang-(1-7)-Mas signaling mediates vasodilation, anti-inflammation, anti-fibrosis, and anti-apoptosis and thus has protective effects in many end-organ tissues. These pathways are normally kept in tight regulatory balance. Thus, ACE2 protects against RAAS-mediated pathogenesis by limiting Ang II substrate availability for the inflammatory ACE-Ang II-AT1R axis and increasing Ang-(1-7) substrate availability for the protective ACE2-Ang-(1-7)-Mas receptor axis.

In an analogous fashion, ACE2 is also the enzyme that cleaves bradykinin and thus is the primary regulator of the balance between pro-inflammatory des-arg⁹-bradykinin binding to kinin B1 receptor (B1R) and cleavage to “inactive peptides” such as bradykinin 1-5 (known also as [1-5]BK and as RPPGF). The heptapeptide Angiotensin (1-7) also potentiates bradykinin action on B2 receptors (Fernandes L., Hypertension. 2001 February; 37(2 Pt 2):703-9). By analogy, it is likely that the supposedly inactive des-arg⁹-bradykinin breakdown products such as bradykinin 1-5 bind to B2R and thus have anti-inflammatory effect.

If any perturbation of this balance occurs then inflammation, proliferation, and cell destruction may ensue, such as via SARS-CoV-2 occupancy and internalization of host cell surface ACE2 with resultant diminution of cell surface ACE2 and consequent unbalanced activation of the inflammatory ACE-Ang II-AT1R pathway. Thus, an absolute or relative deficiency of ACE2 will result in a hyper-inflammatory immune response by a variety of mechanisms, including at least: excess binding of Ang II to AT1R, excess binding of des-arg⁹-bradykinin to BR1, deficient binding of Ang 1-7 to Mas, and potentially deficient binding of Ang 1-7 and possibly des-arg⁹-bradykinin cleavage products to BR2.

There remains a need in the art for therapies to treat or prevent infections caused by coronaviruses, particularly SARS-CoV-2, which has already caused about four million deaths worldwide. Further, there remains a need in the art to treat or prevent diseases and disorders associated with chronic activation of the inflammatory Ang II-AT1R pathway or the des-arg⁹-bradykinin-B1R pathways, both of which are modulated by ACE2 and which may be characterized by either absolute ACE2 deficiency or relative deficiency as measured by increased Angiotensin II or increased des-arg⁹-bradykinin levels.

The present disclosure provides ACE2-Fc fusion proteins. The ACE2-Fc fusion proteins described herein can bind coronavirus viral spike protein and reduce viral entry and replication in host cells while allowing endogenous ACE2 to remain functional. In some embodiments, the ACE2-Fc fusion proteins described herein are beneficial for the treatment of RAAS-mediated diseases (e.g., hypertension) by promoting cleavage of Ang II and other ACE2 ligands.

In some embodiments, the present disclosure provides an angiotensin converting enzyme 2 (ACE2) Fc fusion protein comprising: an ACE2 extracellular domain or fragment thereof and one or more Fc domains.

In some embodiments, the present disclosure provides a homodimeric angiotensin converting enzyme 2 (ACE2) Fc fusion protein comprising a first and a second polypeptide monomer, wherein each monomer comprises an ACE2 extracellular domain or fragment thereof and one or more Fc domain monomers, wherein the one or more Fc domain monomers in the first polypeptide monomer associate with the one or more Fc domain monomers in the second polypeptide monomer to form one or more Fc domains.

In some embodiments, the present disclosure provides a heterodimeric angiotensin converting enzyme 2 (ACE2) Fc fusion protein comprising a first and a second polypeptide monomer, wherein each monomer comprises an ACE2 extracellular domain or fragment thereof and one or more Fc domain monomers, wherein the one or more Fc domain monomers in the first polypeptide monomer associate with the one or more Fc domain monomers in the second polypeptide monomer to form one or more Fc domains.

In some embodiments, the present disclosure provides an angiotensin converting enzyme 2 (ACE2) Fc fusion protein comprising: an ACE2 extracellular domain fragment and one or more Fc domains, wherein the one or more Fc domains demonstrate reduced binding to one or more low affinity Fcγ receptors compared to a wild type IgG1 Fc domain.

In some embodiments, the present disclosure provides an angiotensin converting enzyme 2 (ACE2) Fc fusion protein comprising: an ACE2 extracellular domain fragment and one or more Fc domains, wherein the ACE2 extracellular domain fragment demonstrates increased peripheral tissue penetration relative to the full length ACE2 extracellular domain, such as may be assessed in urine or bronchoalveolar lavage fluid. In some embodiments, the one or more Fc domains of this ACE2 Fc fusion protein provides additive or even synergistic peripheral tissue penetration to the ACE2 ECD fragment comprised herein.

In some embodiments, the present disclosure provides a dimeric angiotensin converting enzyme 2 (ACE2) Fc fusion protein comprising a first and a second polypeptide monomer, wherein each monomer comprises: an ACE2 extracellular domain fragment and one or more Fc domain monomers, wherein the one or more Fc domain monomers in the first polypeptide monomer associate with the one or more Fc domain monomers in the second polypeptide monomer to form one or more Fc domains, and wherein the one or more Fc domains demonstrate reduced binding to one or more low affinity Fcγ receptors compared to a wild type IgG1 Fc domain.

In some embodiments, the present disclosure provides a dimeric angiotensin converting enzyme 2 (ACE2) Fc fusion protein comprising a first and a second polypeptide chain, wherein the first polypeptide chain comprises an ACE2 extracellular domain or ligand-binding fragment thereof, and a first Fc domain monomer polypeptide chain; and the second polypeptide chain comprises an Fc domain monomer polypeptide chain. In some embodiments, the second polypeptide chain further comprises an ACE2 extracellular domain or ligand-binding fragment thereof. In some embodiments, the first Fc domain monomer polypeptide chain and the second Fc domain monomer polypeptide chain of the second polypeptide chain form an Fc domain. In some embodiments, the ACE2 Fc fusion protein is a homodimer.

In some embodiments, the one or more Fc domains demonstrate reduced binding to one or more low affinity Fcγ receptors compared to a wild type IgG1 Fc domain. In some embodiments, the one or more Fc domains are IgG4. In some embodiments, the one or more Fc domains are IgG1 or IgG3 Fc domains that have been mutated to reduce binding to one or more low affinity Fcγ receptors.

In some embodiments, the ACE2 extracellular domain comprises an amino acid sequence that is at least 90%, 91%, 92%, 93%, 94%, or 95% identical to SEQ ID NO: 6. In some embodiments, the ACE2 extracellular domain fragment comprises an amino acid sequence that is at least 90%, 91%, 92%, 93%, 94%, or 95% identical to of SEQ ID NO: 8. In some embodiments, the ACE2 extracellular domain is a ligand-binding fragment thereof. In some embodiments, the ACE2 extracellular domain fragment comprises an amino acid sequence that is at least 90%, 91%, 92%, 93%, 94%, or 95% identical to of SEQ ID NO: 8. In some embodiments, the ACE2 extracellular domain or fragment thereof further comprises a signal peptide of SEQ ID NO: 2. In some embodiments, the signal peptide is cleaved from the mature protein.

In some embodiments, the ACE2 extracellular domain or fragment thereof comprises one or more point mutations. In some embodiments, the one or more point mutations in the ACE2 extracellular domain or fragment thereof decreases the formation of higher-order multimers or aggregates. In some embodiments, the one or more point mutations in the ACE2 extracellular domain or fragment thereof decreases binding to angiotensin II and/or decreases enzymatic activity when bound to angiotensin II. In some embodiments, the one or more point mutations in the ACE2 extracellular domain or fragment thereof increases binding to viral spike protein.

In some embodiments, the ACE2 extracellular domain or fragment thereof comprises a point mutation at position 82 of SEQ ID NO: 5 or SEQ ID NO: 7. In some embodiments, the ACE2 extracellular domain or fragment thereof comprises the point mutation M82A, M82D, M82N, or M82S.

In some embodiments, the ACE2 extracellular domain or fragment thereof comprises a point mutation at position 30 of SEQ ID NO: 5 or SEQ ID NO: 7. In some embodiments, the ACE2 extracellular domain or fragment thereof comprises the point mutation D30E or D30Q.

In some embodiments, the ACE2 extracellular domain or fragment thereof comprises a point mutation at position 31, 34, and/or 38 of SEQ ID NO: 5 or SEQ ID NO: 7. In some embodiments, the ACE2 extracellular domain or fragment thereof comprises a K31T point mutation. In some embodiments, the ACE2 extracellular domain or fragment thereof comprises a H34Q point mutation. In some embodiments, the ACE2 extracellular domain or fragment thereof comprises a D38E point mutation. In some embodiments, the ACE2 extracellular domain or fragment thereof comprises one or more point mutations selected from the group consisting of D30E, K31T, H34Q, and D38E.

In some embodiments, the ACE2 extracellular domain or fragment thereof comprises a point mutation at position 139 of SEQ ID NO: 5 or SEQ ID NO: 7. In some embodiments, the ACE2 extracellular domain or fragment thereof comprises the point mutation Q139A, Q139S, or Q139V.

In some embodiments, the ACE2 extracellular domain or fragment thereof comprises a point mutation at position 175 of SEQ ID NO: 5 or SEQ ID NO: 7. In some embodiments, the ACE2 extracellular domain or fragment thereof comprises the point mutation Q175A, Q175S, or Q175V.

In some embodiments, the ACE2 extracellular domain or fragment thereof comprises a point mutation at position 374 and/or position 378 of SEQ ID NO: 5 or SEQ ID NO: 7. In some embodiments, the ACE2 extracellular domain or fragment thereof comprises the point mutation H374S, H374A, or H374V and/or H378S, H378A, or H378V. In some embodiments, the ACE2 extracellular domain or fragment thereof comprises the point mutations M82N, Q139A, H374S, and H378S.

In some embodiments, the Fc domain is an IgG1 Fc domain. In some embodiments, the IgG1 Fc domain comprises an IgG1 hinge, an IgG1 CH2 domain, and an IgG1 CH3 domain. In some embodiments, the IgG1 Fc domain comprises an amino acid sequence of SEQ ID NO: 39. In some embodiments, the Fc domain is an IgG4 Fc domain. In some embodiments, IgG4 Fc domain comprises an IgG4 hinge, an IgG4 CH2 domain, and an IgG4 CH3 domain. In some embodiments, the IgG4 Fc domain comprises an amino acid sequence of SEQ ID NO: 42.

In some embodiments, the ACE2-Fc fusion protein further comprises a signal peptide, wherein the signal peptide comprises an amino acid sequence of SEQ ID NO: 2. In some embodiments, the signal peptide is cleaved from the ACE2-Fc fusion protein. In some embodiments, the signal peptide is cleaved between amino acid positions 17 and 18 of the ACE2-Fc fusion protein comprising the amino acid sequence selected from the group consisting of SEQ ID NO: 43-50. In some embodiments, the signal peptide is cleaved between amino acid positions 19 and 20 of the ACE2-Fc fusion protein comprising the amino acid sequence selected from the group consisting of SEQ ID NO: 43-50.

In some embodiments, the ACE2 extracellular domain or fragment thereof comprises an amino acid sequence selected from the group consisting of SEQ ID NOs: 6, 8-38, and 51. In some embodiments, the ACE2-Fc fusion protein comprises an amino acid sequence selected from the group consisting of SEQ ID NOs: 52-59. In some embodiments, the ACE2-Fc fusion protein comprises an amino acid sequence of SEQ ID NO: 59. In some embodiments, the ACE2-Fc fusion protein comprises an amino acid sequence of SEQ ID NO: 50. In some embodiments, the signal peptide of SEQ ID NO:2 is cleaved from the mature protein.

In some embodiments, the ACE2-Fc fusion protein forms a homodimer.

In some embodiments, the ACE2-Fc fusion protein binds to a coronavirus spike protein. In some embodiments, the ACE2-Fc fusion protein binds to the coronavirus spike protein with Kd of about 1 nM to about 100 nM. In some embodiments, the coronavirus is SARS-CoV-1 or SARS-CoV-2. In some embodiments, the coronavirus is SARS-CoV-1 variant or SARS-CoV-2 variant.

In some embodiments, the ACE2-Fc fusion protein binds and cleaves an ACE2 ligand. In some embodiments, the ACE2 ligand is angiotensin I, angiotensin II, apelin, pro-dynorphin, or des-arg⁹-bradykinin.

In some embodiments, the ACE2-Fc fusion protein demonstrates one or more of the following characteristics: (i) transport into the extracellular space via interaction with the FcRn; (ii) prolonged circulating half-life in human (e.g., greater than 24, 48, 72, or 96 hours); (iii) providing replacement ACE2 enzymatic activity in subjects with increased Angiotensin II; and (iv) reduced likelihood of Antibody Dependent Enhancement (ADE) compared to a fusion protein with an Fc domain that binds to low affinity Fc receptors.

In some embodiments, the present disclosure provides a recombinant polynucleotide encoding a monomer of the ACE2-Fc fusion protein described herein. In some embodiments, the recombinant polynucleotide further comprises a nucleic acid sequence encoding a signal peptide.

In some embodiments, the present disclosure provides an expression vector comprising the recombinant polynucleotide described herein. In some embodiments, the present disclosure provides a host cell comprising the expression vector described herein.

In some embodiments, the present disclosure provides method of treating or preventing one or more diseases or disorders, the method comprising administering the ACE2-Fc fusion protein described herein to a subject in need thereof. In some embodiments, the subject is human.

In some embodiments, the ACE2-Fc fusion protein is administered once per day, once per week, or multiple times per day or per week. In some embodiments, the ACE2-Fc fusion protein is administered at dose of about 0.001 mg/kg to about 1000 mg/kg of body weight per day. In some embodiments, the ACE2-Fc fusion protein is administered intravenously, subcutaneously, orally, intraperitoneally, or intramuscularly.

In some embodiments, the one or more diseases or disorders is caused by a coronavirus. In some embodiments, the coronavirus is SARS-CoV-1 or SARS-CoV-2. In some embodiments, the coronavirus is a SARS-CoV-1 variant or a SARS-CoV-2 variant.

In some embodiments, the one or more diseases or disorders is selected from the group consisting of cardiovascular disease, hypertension, cardiopulmonary disease, acute lung injury, acute respiratory distress syndrome, pulmonary fibrosis, diabetes-related micro- and macro-vascular diseases, metabolic syndrome, stress-related disorders, liver disease, kidney disease, ocular disorders, endometriosis, a neurodegenerative disease, an endocrine disorder, a granulomatous disease, a non-granulomatous disease, arthritis, cancer, sepsis, a mood or anxiety disorder, inflammation and autoimmunity.

In some embodiments, the ACE2-Fc fusion protein has an EC50 value of less than about 10 μM, less than about 1 μM, less than about 0.1 μM, less than about 0.01 μM, or less than about 0.001 μM when assaying binding of ACE2 to viral spike proteins.

In some embodiments, the present disclosure provides a composition comprising a plurality of ACE2-Fc fusion proteins described herein, wherein the composition comprises at least 80% homodimers w/w. In some embodiments, the composition comprises at least 85% w/w, at least 90% w/w at least 95% w/w at least 96% w/w, at least 97% w/w, at least 98% w/w, or at least 99% w/w homodimers.

In some embodiments, the present disclosure provides a method of treating a disease or disorder in a subject in need thereof comprising detecting a level of angiotensin II (Ang II) in the subject and administering the ACE2-Fc fusion protein described herein to the subject if an elevated level of Ang II is detected.

In some embodiments, the present disclosure provides a method of treating a disease or disorder in a subject in need thereof comprising detecting a level of des-arg-9-bradykinin in the subject and administering the ACE2-Fc fusion protein described herein to the subject if an elevated level of des-arg-9-bradykinin is detected.

In some embodiments, the present disclosure provides a method of treating a disease or disorder in a subject in need thereof comprising detecting a level of Ang 1-7 in the subject and administering the ACE2-Fc fusion protein described herein to the subject if a diminished level of Ang 1-7 is detected.

In some embodiments, the present disclosure provides a method of treating a disease or disorder in a subject in need thereof comprising detecting a ratio of Ang II to Ang 1-7 administering the ACE2-Fc fusion protein described herein to the subject if an elevated level Ang II/Ang 1-7 ratio is detected.

In some embodiments, the detected level of Ang II, elevated level of des-arg-9-bradykinin, or elevated ratio of Ang II to Ang 1-7 is elevated relative to the subject's historical level or ratio. In some embodiments, the detected level of Ang II, elevated level of des-arg-9-bradykinin, or elevated ratio of Ang II to Ang 1-7 is elevated relative to the level or ratio detected in a healthy control population.

### BRIEF DESCRIPTION OF THE DRAWINGS

FIGS. 1A and 1B illustrates the general nature of the therapeutics described herein comprising primarily two independently functional ACE2 extracellular domains or fragments thereof and a homodimeric Fc domain. The depiction of the ACE2 extracellular domains specifically includes fragments thereof, including ligand-binding fragments thereof.

FIG. 2 illustrates the role of ACE2 in the renin-angiotensin-aldosterone system (RAAS). ACE2 promotes the anti-inflammatory ACE2/Ang-(1-7)/Mas receptor axis (right panel) while loss of ACE2 increases activity of the pro-inflammatory ACE/Ang II/AT1 receptor axis (left panel).

FIG. 3 illustrates the interaction between SARS-CoV-2 and the Renin-Angiotensin-Aldosterone System (RAAS) in host cells. The SARS-CoV-2 spike protein binds to the ACE2 receptor, resulting in membrane fusion, entry and replication in host cells. As a result of viral entry through endocytosis, surface ACE2 is down-regulated, resulting in unopposed angiotensin II accumulation which binds the angiotensin II type 1 receptor and activates the inflammatory RAAS pathway. Activation of the inflammatory RAAS pathway further mediates pro-inflammatory lung injury responses. ACE denotes the angiotensin-converting enzyme and ARB denotes the angiotensin-receptor blocker.

FIG. 4A shows non-reduced and reduced SD S-PAGE analyses of the ACE2-IgG4 fusion protein GL-4316. Non-reduced SDS-PAGE shows an upper band below 260 kD that corresponds to the homodimeric form of GL-4316 and a lower band at approximately 120 kD that represents the monomeric form of GL-4316. Reduced SDS-PAGE shows a band at approximately 120 kD that corresponds to the monomeric form of GL-4316. FIG. 4B shows size-exclusion chromatography (SEC) of the ACE2-Fc fusion protein GL-4316. SEC revealed one major peak representing the homodimeric form of GL-4316. The right shoulder of the major peak likely represents the monomeric or other partial form of GL-4316 such as a homodimeric Fc with a single ACE2 domain.

FIG. 5 shows the ACE2 enzymatic activity of GL-4316 and an ACE2 positive control at dilutions of 50×, 100×, or 200×.

FIG. 6 shows the concentration of GL-4316 in the serum of rats following intravenous or subcutaneous treatment with 20 mg/kg or 60 mg/kg of GL-4316.

FIG. 7A shows GL-4316 levels (left panel) and ACE2 enzymatic activity (right panel) in the bronchoalveolar lavage fluid (BALF) of rats at 24 hours post-treatment with 100 mg/kg of GL-4316. FIG. 7B shows GL-4316 levels in urine of rats 24 hours post-treatment with 100 mg/kg of GL-4316.

FIG. 8 demonstrates the pharmacokinetic profile of GL-4316 injected subcutaneously and intravenously in cynomolgus monkeys.

FIG. 9A shows binding of the ACE2-IgG4 fusion protein, GL-4316, to SARS-CoV-2 S1 spike protein by ELISA. FIG. 9B shows binding of GL-4316 supernatant comprised primarily of homodimers and purified protein comprised of multimers to SARS-CoV-2 S1 spike protein by ELISA. The SDS-PAGE gel shows that GL-4316 presents as a homodimer at approximately 260 kD in cell-culture supernatant and can be manufactured to multimerize and to aggregate. FIG. 9C shows binding of GL-4316 to wild-type (D614) or mutant (D613G) SARS-CoV-2 S1 spike protein by ELISA.

FIG. 10A shows binding curves of human IgG1 Fc (rFc) and ACE2-IgG4 fusion protein (GL-4316) to SARS-CoV-2 S1 protein as determined by biolayer interferometry (Octet). FIG. 10B shows binding curves of GL-4316 by Octet to the original Wuhan SARS-CoV-2 and to several pathogenic mutants.

FIG. 11 shows a schematic of the Focus Reduction Neutralization Assay. The left panel (“A”) shows that in the absence of a neutralizing compound, primary anti-SARS antibodies bind to the virus' protein. A secondary antibody (horseradish peroxidase anti-human IgG) binds to the primary antibody and a substrate (TrueBlue) is then added, which binds to the secondary antibody and generates blue spots that are visualized and imaged on an ELISpot reader (control sample). The right panel (“B”) shows that a neutralizing compound will bind to SARS-CoV-2 resulting in a virus/antibody complex that prevents the virus from infecting Vero cells, and an absence of blue spots.

FIG. 12A shows representative panels of the Focus Reduction Neutralization Assay in Vero cells infected with SARS CoV-2 virions. SARS-CoV-2 infected cells were either left untreated (positive control) or treated with ACE2-IgG4 fusion protein (GL-4316) or the convalescent serum control.

FIG. 12B shows the percent inhibition of SARS-CoV-2 fusion/entry in Vero cells with treatment of increasing concentrations of ACE2-IgG4 fusion protein (GL-4316). The 50 and 90% effective concentration (EC_{50/90}) of GL-4316 required to inhibit viral protein expression was calculated by nonlinear regression analysis. The Focus Reduction Neutralization Assay was repeated in two independent experiments and the percent inhibition values of SARS-CoV2 (relative to untreated control) were plotted in the graph (mean±SD). Treatment with GL-4316 resulted in complete inhibition of viral entry into cells (EC₁₀₀).

FIG. 13 shows the gross lung pathology of Golden Syrian hamsters treated with GL-4316 or PBS in a semi-therapeutic model where drug is administered on the same day as SARS-CoV-2 infection compared both to an uninfected control and to an infected PBS control.

FIG. 14A shows the diminished weight loss of Syrian hamsters treated with GL-4316 in a prophylactic model where drug administration begins 1 day before SARS-CoV-2 inoculation compared both to an uninfected control and to an infected PBS control. Uninfected hamsters exhibited normal weight gain. FIG. 14B is the same data as FIG. 14A re-graphed to focus on the treatment arms and treatment days only. FIG. 14C shows representative sections of lung tissue from the 70 mg/Kg and PBS cohorts of the prophylactic Syrian hamster SARS-CoV-2 model. FIG. 14D shows that in this Syrian hamster prophylactic model which demonstrates preservation from weight loss, the percent of lung with proliferation and/or inflammation is significantly reduced in a dose-responsive manner when assessed by a histopathologist masked to treatment cohort. FIG. 14E shows representative sections of lung vascular tissue sections from the prophylactic Syrian hamster SARS-CoV-2 model identifying both normal lung vasculature and six attributes of diseased lung vasculature. FIG. 14F shows that in this prophylactic model which demonstrates preservation from weight loss, the scoring of lung vasculature damage on 6 criteria significantly suppresses SARS-CoV-2 induced lung intravascular and perivascular damage and vasculitis in a dose-responsive manner when assessed by a histopathologist masked to treatment cohort. FIG. 14G shows that in this prophylactic model which demonstrates preservation from weight loss, the scoring of lung intramural vascular damage on 3 criteria significantly suppresses SARS-CoV-2 induced lung intramural vascular damage in a dose-responsive manner when assessed by a histopathologist masked to treatment cohort.

FIG. 15 shows the binding curves of ACE2-IgG1 Fc fusion proteins comprising an ACE2 extracellular domain fragment and mutational variants thereof and an IgG1 Fc domain. GL-ACE2-IgG1 is the reference (parental) ACE2-IgG1 fusion protein. The figure demonstrates that a number of mutational ACE2-IgG1 Fc fusion protein variants as tested have a lower EC50 than the IgG1 parental ACE2-IgG1 fusion protein.

FIG. 16A-FIG. 16D show the binding of G001 and GL-4316 to high affinity FcγRT (FIG. 16A) and to low affinity FcγRs, FcγRIIA (FIG. 16B), FcγRIM (FIG. 16C), FcγRIIIA (FIG. 16D). The data demonstrate that GL-4316 binds similarly to G001 to FcγR1 and not at all to low affinity FcγRs.

FIG. 17 shows the binding of G001 and GL-4316 to neonatal receptor FcRn. The data demonstrate that at both neutral pH and pH 6.0, GL-4316 binds similarly to G001 to neonatal receptor.

FIG. 18 shows the binding of GL-4316 to the original SARS-CoV-2 Wuhan spike protein and to three important pathogenic mutant strains—WHO alpha (B1.1.7), beta (B.1351), and gamma (P.1). The binding was assessed on a MesoScale Discovery instrument using MSD commercial plates containing the relevant spike protein with Electrochemiluminescence (ECL) readout. The data reveal no diminution in binding of GL-4316 to any pathogenic mutant SARS-CoV-2 spike protein.

### DETAILED DESCRIPTION

### Definitions

The present disclosure is described herein using several definitions, as set forth below and throughout the description.

As used in this specification and the appended claims, the singular forms “a,” “an” and “the” include plural references unless the context clearly dictates otherwise.

As used in this specification, the term “and/or” is used in this disclosure to either “and” or “or” unless indicated otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage sequence identity is calculated by determining the number of positions at which the identical nucleic acid base or amino acid residue occurs in both sequences to yield the number of identical positions. The number of identical positions is then divided by the total number of positions in the comparison window and multiplied by 100 to yield the percentage of sequence identity. Percentage of sequence identity is determined by comparing two optimally aligned sequences over a comparison window. The comparison window for polynucleotide sequences can be, for instance, at least 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option.

In some embodiments, a “variant” of a particular polypeptide sequence may be defined as a polypeptide sequence having at least 20% sequence identity to the particular polypeptide sequence over a certain length of one of the polypeptide sequences using blastp with the “BLAST 2 Sequences” tool. Such a pair of polypeptides may show, for example, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length of one of the polypeptides, or range of percentage identity bounded by any of these values (e.g., range of percentage identity of 80-99%).

A “fragment” is a portion of an amino acid sequence which is identical in sequence to but shorter in length than a reference sequence (e.g., a fragment of the ACE2 extracellular domain). A fragment may comprise up to the entire length of the reference sequence, minus at least one amino acid residue. For example, a fragment may comprise from 5 to 1000 contiguous amino acid residues of a reference polypeptide. In some embodiments, a fragment may comprise at least 5, 10, 15, 20, 25, 30, 40, 50, 60, 70, 80, 90, 100, 150, 250, or 500 contiguous amino acid residues of a reference polypeptide; or a fragment may comprise no more than 5, 10, 15, 20, 25, 30, 40, 50, 60, 70, 80, 90, 100, 150, 250, or 500 contiguous amino acid residues of a reference polypeptide; or a fragment may comprise a range of contiguous amino acid residues of a reference polypeptide bounded by any of these values (e.g., 40-80 contiguous amino acid residues). Fragments may be preferentially selected from certain regions of a molecule. For example, the ACE2 extracellular domain or fragment thereof comprising the amino acid sequence of SEQ ID NO: 8 is a fragment of ACE2 extracellular domain reference sequence SEQ ID NO: 6. A “variant” of a reference polypeptide sequence may include a fragment of the reference polypeptide sequence.

As used herein, the term “optimized” refers to an ACE2-Fc fusion protein that has been improved over the parental ACE2-Fc fusion protein. In some embodiments, the ACE2-Fc fusion protein is optimized through one or more point mutations in the ACE2 extracellular domain and/or Fc domain.

As used herein, the term “Fc domain” refers to a polypeptide sequence corresponding to or derived from the portion of an antibody that is capable of binding to Fc receptors on cells and/or the Clq component of complement, thereby mediating the effector function of an antibody. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc domain is a homodimeric protein comprising two polypeptides that are associated by disulfide bonds, and each comprising a hinge region, a CH2 domain, and a CH3 domain. However, more recently the term has been applied to the single chain monomer component consisting of CH3, CH₂, and at least a portion of the hinge sufficient to form a disulfide-linked dimer with a second such chain which may be a homodimer or a heterodimer. Herein, the term “Fc domain” refers to the dimeric form of the Fc domain. The term “Fc domain monomer” refers to the individual monomers that associate to form the dimeric protein. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc domain includes variants of naturally occurring sequences.

The term “immunoglobulin constant region” or “constant region” refers to a peptide or polypeptide sequence that corresponds to or is derived from part or all of one or more constant domains of an immunoglobulin (e.g., CH1, CH2, CH3). Depending on the context, use of the term “immunoglobulin constant region” may refer to either the dimeric form of the protein or the individual monomers that associate to form the dimeric protein. The term “immunoglobulin heavy chain constant region” (also referred to as “heavy chain constant region” or “CH”) refers to the constant region from the antibody heavy chain. The CH is further divisible into CH1, CH2, and CH3 domains (e.g., IgA, IgD, or IgG isotypes), or CH1, CH2, CH3, and CH4 domains (e.g., IgE or IgM isotypes). In some embodiments, the heavy chain constant domains are part or all of IgG1 constant domains. In some embodiments, the constant domains are part or all of IgG4 constant domains. In some embodiments, the constant domains making up the constant region are human.

The term “bispecific antibody” or “bispecific molecule” refers to a compound that can bind to two different antigens at the same time. In some embodiments, the ACE2 fusion protein is a bispecific molecule comprising an ACE2 extracellular domain that binds an ACE2 ligand and an antigen binding-arm that binds a different antigen (e.g., an antigen that increases delivery of the ACE2-Fc fusion protein to sites of infection and/or inflammation).

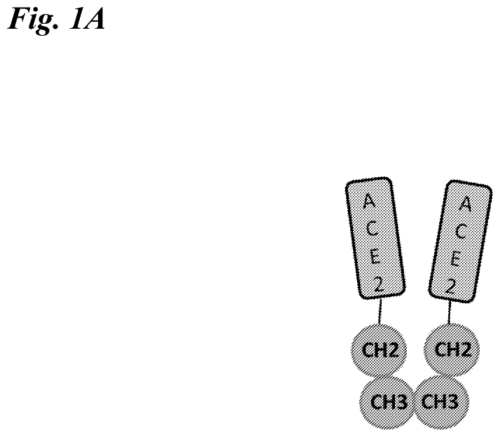

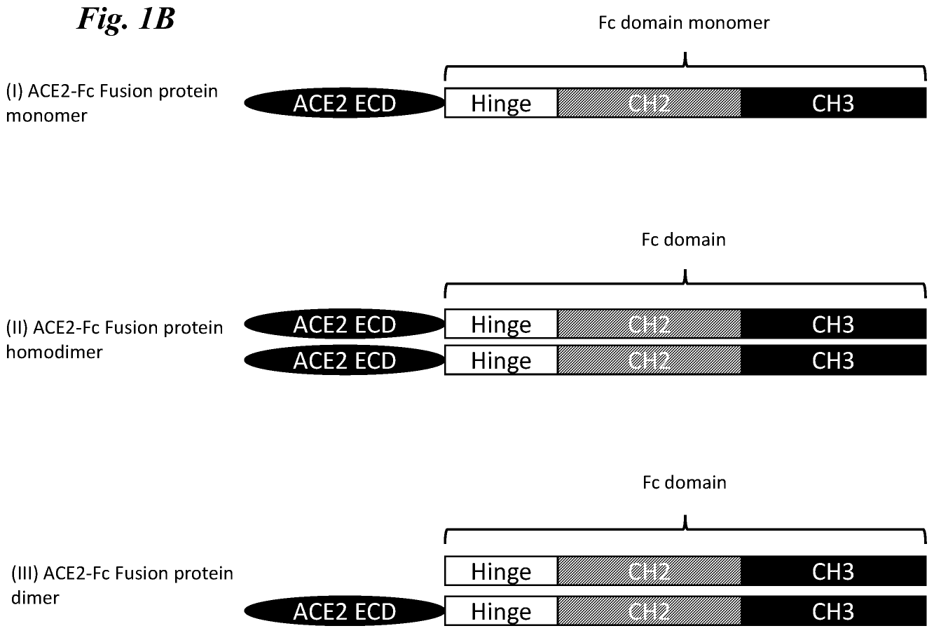

As used herein, a “polypeptide” or “protein” refers to a single, linear, and contiguous arrangement of covalently linked amino acids. Polypeptides can form one or more intrachain disulfide bonds. The terms polypeptide and protein also encompass embodiments where two polypeptide chains link together in a non-linear fashion, i.e. dimerize, such as via an interchain disulfide bond. For example, a single-chain polypeptide or ACE2-Fc fusion protein monomer comprises an ACE2 extracellular domain or fragment thereof and one or more Fc domain monomers. Two ACE2-Fc fusion protein monomers linked together, i.e., two polypeptide chains, form an ACE2-Fc fusion protein dimer. The ACE2-Fc fusion protein dimer can be a homodimer comprising two ACE2 extracellular domains or fragments thereof and one functional Fc domain (See FIG. 1A and FIG. 1B(II)), or a heterodimer comprising one ACE2 extracellular domain and a functional Fc domain (See FIG. 1B(III)) or fragments thereof and one functional Fc domain. As used herein the term “dimer” refers to a protein comprised of two monomers wherein the monomers are either the same (“homodimer”) or different (“heterodimer”).

The term “higher-order multimer” refers to an ACE2-Fc fusion protein comprising more than three ACE2-Fc fusion protein dimers associated or linked together. Higher-order ACE2-Fc fusion proteins may be trimers of a homodimer, tetramers of a homodimer, pentamers of a homodimer, hexamers of a homodimer, and above.

The amino acid sequences contemplated herein may include one or more amino acid substitutions relative to a reference amino acid sequence. For example, the ACE2 extracellular domain or fragment thereof comprising the native human amino acid sequence of SEQ ID NO: 8 may comprise one or more amino acid substitutions or point mutations.

The term “dissociation constant” or “Kd” refers to a dissociation equilibrium constant of a particular interaction between a first protein or peptide and a second protein or peptide (e.g., an ACE2-Fc fusion protein and a viral spike protein).

The disclosed methods and compositions described herein may be used to treat or prevent a disease of disorder in a subject in need thereof. A “subject in need thereof” includes a subject having or at risk of being infected by a microorganism that binds to ACE2, in particular a virus, and especially a coronavirus, e.g., SARS-CoV-2. A “subject in need thereof” includes a subject having or at risk for developing diseases and disorders such as diabetic and non-diabetic chronic kidney disease, acute renal failure, glomerulonephritis, hypertension, scleroderma, pulmonary hypertension, acute lung injury, renovascular hypertension secondary to renal artery stenosis, idiopathic pulmonary fibrosis, liver fibrosis such as in liver cirrhosis, an aortic aneurysm, cardiac fibrosis and remodeling, left ventricular hypertrophy, autoimmune or inflammatory disease, endometriosis, and an acute stroke. A “subject in need thereof” includes a subject that has elevated expression of angiotensin II, decreased expression of ACE2, and/or chronic activation of the inflammatory AT1R pathway.

The term “half-life” or “T1/2” refers to the time taken for half the initial dose of an ACE2-Fc fusion protein administered to a subject to be eliminated from the body.

The term “IC50” or “IC₅₀” refers to the half-maximal inhibitory concentration of an ACE2-Fc fusion protein as measured using in an in vitro assay. The term “EC50” or “EC₅₀” is refers to the half-maximal cytotoxicity concentration of the ACE2-Fc fusion protein in an in vitro cytotoxicity assay or an in-vitro assay.

### Overview

The present disclosure provides ACE2-Fc fusion proteins, compositions thereof, and methods of use in the treatment of a variety of diseases. In some embodiments, the disease are associated with chronic activation of the inflammatory Ang II-AT1R pathway or the des-arg⁹-bradykinin-B1R pathway, both of which are modulated by ACE2 and which may be characterized by either absolute ACE2 deficiency or relative deficiency as measured by increased Angiotensin II levels. In some embodiments, the present disease is an infectious by a coronavirus, particularly SARS-CoV-2.

Monoclonal antibody combinations have been rapidly brought to market based on an ability to bind SARS-CoV-2 spike protein and thereby neutralize the virus (See, J. Hansen et al., Science 10.1126/science.abd0827; 2020; P. Chen et al. N Engl J Med. 2021 Jan. 21; 384(3):229-237). Such monoclonal antibodies include sotrovimab (GlaxoSmithKline), casirivimab (Regeneron), imdevimab (Regeneron), bamlanivimab (Eli Lilly and Company), etesevimab (Eli Lilly and Company), administered alone or in combination. However, selective pressure and consequent viral mutations have already decreased or in some cases eliminated the antiviral activity of these monoclonal antibodies, resulting in regulators already withdrawing marketing authorization for bamlanivimab monotherapy.

The ACE2-Fc fusion proteins described herein provide advantages over the existing monoclonal antibody treatments as they are not dependent on binding to viral protein epitopes that are capable of mutation and are therefore effective against multiple viral variants. Therefore, the ACE2-Fc fusion proteins described herein are capable of binding to and neutralizing virus that is not effectively bound and neutralized by available monoclonal antibodies and antibody combinations. In this way, the ACE2-Fc fusion proteins described herein are capable of binding and neutralizing, and thereby treating infection by, all pathogenic SARS-CoV-2 strains. In some embodiments, the ACE2-Fc fusion proteins described herein are at least as efficacious as monoclonal antibodies in treating the SARS-CoV-2 infection. In some embodiments, the ACE2-Fc fusion proteins described herein are efficacious against all SARS-CoV-2 variants that bind to ACE2.

Furthermore, in some embodiments, the ACE2-Fc fusion proteins described herein comprise an Fc domain that demonstrates reduced binding to low affinity Fc receptors, and thereby reduces the likelihood of Antibody Dependent Enhancement (ADE). Additional advantages of the ACE2-Fc fusion proteins described herein include: (i) transport into the extracellular space via interaction with the FcRn; (ii) prolonged circulating half-life in human (e.g., greater than 24, 48, 72, or 96 hours); (iii) providing replacement ACE2 enzymatic activity in subjects with increased Angiotensin II. Therefore, in some embodiments, the ACE2-Fc fusion proteins described herein demonstrate one or more of (i) transport into the extracellular space via interaction with the FcRn; (ii) prolonged circulating half-life in human (e.g., greater than 24, 48, 72, or 96 hours); (iii) providing replacement ACE2 enzymatic activity in subjects with increased Angiotensin II; and (iv) reduced likelihood of ADE compared to a fusion protein with an Fc domain that binds to low affinity Fc receptors.

Components and domains of the ACE2-Fc fusion proteins are described below, and exemplary formats of the ACE2-Fc fusion proteins are illustrated in FIG. 1B. The smallest functional unit of the ACE2-Fc fusion proteins described herein is an ACE2-Fc fusion protein monomer exemplified in (I) of FIG. 1B (i.e., a single chain polypeptide comprising an Fc domain monomer and an ACE2 extracellular domain or fragments thereof). The ACE2-Fc fusion protein monomers are biologically active as the ACE2 domain is enzymatically active, but these monomers do not comprise a dimeric Fc domain. In particular embodiments, the ACE2-Fc fusion protein monomers associate with a second ACE2-Fc fusion protein monomers to form a homodimer comprising a functional dimeric Fc domain and two ACE2 extracellular domains. This homodimer is exemplified in (II) of FIG. 1B. These homodimers are capable of binding to ACE2 ligands with avidity by virtue of the two ACE2 ECDs. In some embodiments, the ACE2-Fc fusion protein monomers associate with an Fc domain monomer to form a dimer comprising a functional dimeric Fc domain and one ACE2 extracellular domain. This dimer is exemplified in (III) of FIG. 1B. In some embodiments, compositions of the ACE2-Fc fusion proteins described herein comprise less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1% of these ACE2-Fc fusion protein dimers exemplified in FIG. 1B (III) and/or of the ACE2-Fc fusion protein monomers exemplified in FIG. 1B (I). See, Handlogten et al., mAbs, 2020; 12, Article No. 1779974. In some embodiments, the ACE2-Fc fusion proteins multimerize to form ACE2-Fc fusion protein multimers comprising two or more ACE2-Fc fusion proteins (e.g., dimers of dimers, trimers of dimers, tetramers of dimers, pentamers of dimers, hexamers of dimers, and above). Unless otherwise indicated, reference to an “ACE2-Fc fusion protein” throughout this specification refers to a dimeric form of the fusion protein (i.e., an ACE2-Fc fusion protein comprising a dimeric Fc domain as illustrated in FIG. 1B (II) and (III)).

In some embodiments, the present disclosure provides an angiotensin converting enzyme 2 (ACE2) Fc fusion protein comprising a dimer that comprises a first and a second polypeptide chain, wherein the first polypeptide chain comprises an ACE2 extracellular domain or ligand-binding fragment thereof, and a first Fc domain monomer polypeptide chain (FIG. 1B (I)); and the second polypeptide chain comprises a second Fc polypeptide domain monomer polypeptide chain (FIG. 1B (III)). In some embodiments, the second polypeptide chain further comprises an ACE2 extracellular domain or ligand-binding fragment thereof (FIG. 1B (II)). In such embodiments, the first and second Fc domain monomer polypeptide chains form an Fc domain. In some embodiments, the ACE2-Fc fusion protein is a homodimer (FIG. 1B (II)). In some embodiments, the ACE2-Fc fusion protein is a heterodimer (FIG. 1B (III)). In some embodiments, the present disclosure provides a multimer comprising at least two ACE2-Fc fusion proteins described herein. In some embodiments, the ACE2 Fc fusion protein is a single chain monomer polypeptide comprising an ACE2 extracellular domain or ligand-binding fragment thereof and an Fc domain monomer (FIG. 1B (I)). In some embodiments, the ACE2 Fc fusion protein monomer retains ACE2 enzymatic activity.

### Signal Peptides

In some embodiments, the ACE2-Fc fusion proteins of the present disclosure comprise a signal peptide. As used herein, the term “signal peptide” refers to the leader sequence ensuring entry into the secretory pathway. In some embodiments, the signal peptide is directly linked to the ACE2 domain or fragment or variant thereof. In some embodiments, the signal peptide is cleaved from the mature ACE2-Fc fusion protein. In some embodiments, the signal peptide is cleaved from the mature ACE2-Fc fusion protein at a point different from the native human signal peptide and may result in a mature protein of a different amino acid length (e.g., 1, 2, 3, 4, 5, 6, 7, 8, 9, or 10 amino acid difference). In some embodiments, the mature protein with a different amino acid length due to the alternative signal peptide cleavage site exhibits increased or decreased binding to ACE2 ligands.

Secreted proteins are expressed initially inside the cell in a precursor form containing a leader sequence ensuring entry into the secretory pathway. Such leader sequences, named signal peptides, direct the expressed product across the membrane of the endoplasmic reticulum (ER). Signal peptides are generally cleaved by signal peptidases during translocation to the ER. Once in the ER, the mature protein is transported to the Golgi apparatus and routed out of the cell to be secreted to the external medium (Pfeffer and Rothman (1987) Ann. Rev. Biochem. 56:829-852).

Exemplary signal peptides are show in Table 1 below.

TABLE1
Exemplary Signal Peptides

| Name | Amino Acid Sequence | SEQ ID NO: |
|---|---|---|
| IgG kappa SP | METDTLLLWVLLLWVPGSTG | 1 |
| ACE2 SP | MSSSSWLLLSLVAVTAA | 2 |

In some embodiments, the signal peptide is cleaved from the ACE2-Fc fusion proteins described herein. In some embodiments, the ACE2-Fc fusion protein comprises a signal peptide with the amino acid sequence of SEQ ID NO: 1. In some embodiments, the ACE2-Fc fusion protein comprises a signal peptide with the amino acid sequence of SEQ ID NO: 2.

In some embodiments, the ACE2-Fc fusion protein comprises a signal peptide and the signal peptide is cleaved between amino acid positions 17 and 18 of the ACE2-Fc fusion protein comprising the amino acid sequence selected from the group consisting of SEQ ID NO: 43-50.

In some embodiments, the ACE2-Fc fusion protein comprises a signal peptide and the signal peptide is cleaved between amino acid positions 19 and 20 of the ACE2-Fc fusion protein comprising the amino acid sequence selected from the group consisting of SEQ ID NO: 43-50.

### Angiotensin Converting Enzyme 2 (ACE2)

The ACE2-Fc fusion proteins of the present disclosure comprise angiotensin converting enzyme 2 (ACE2) or fragments or variants thereof, including ligand-binding fragments thereof.

ACE2 belongs to the membrane-bound carboxydipeptidase family and has a multitude of critical functions. ACE2 cleaves the C-terminal residue of a number of peptide substrates, including angiotensin I (Ang I), angiotensin II (Ang II), des-arg⁹-bradykinin, (Danilczyk and Penninger, Circulation Research 2006, (98)4:463-471) and neurotensin 1-13 and kinetensin (Donoghue M. Circ. Res. 2000; 87:E1-E9). In addition, ACE2 hydrolyzes apelin-13 and dynorphin A 1-13 with as high a catalytic efficiency as Ang II (Vickers C. J. Biol. Chem. 2002; 277:14838-14843). Other molecular functions of ACE2 include virus receptor binding activity, endopeptidase activity, glycoprotein binding activity, metallocarboxypeptidase activity, and zinc ion binding activity (See, Batlle and Wysocki, U.S. Pub. No. US2018/0230447).

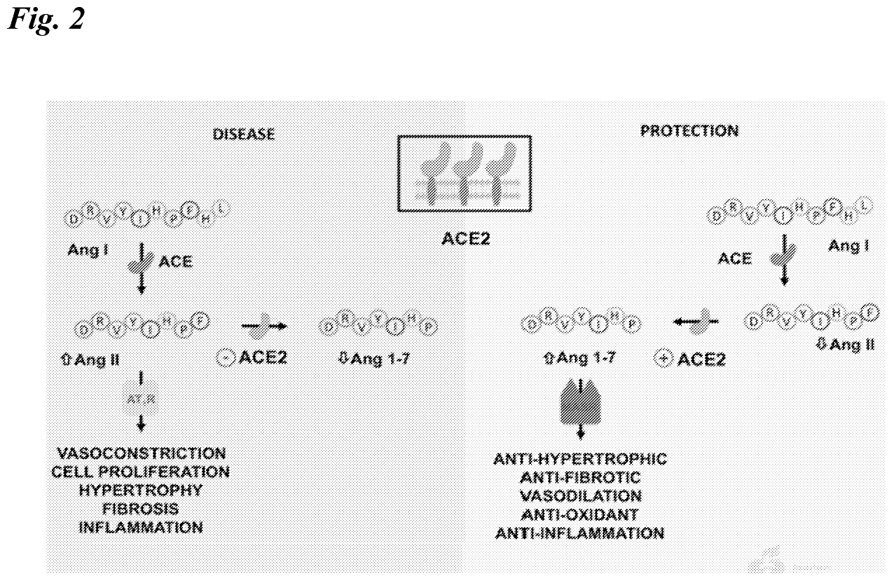

ACE2 plays an important role in regulation of the renin-angiotensin-aldosterone system (RAAS, See FIG. 2). ACE2-mediated cleavage of angiotensin II (Ang II) generates Ang-(1-7), which activates the mas oncogene pathway (P. Verdecchia et al., Eur J IM, 76 (2020) 14-20; P. Domingo et al./EBioMedicine 58 (2020) 102887). The Ang II-Ang-(1-7)-mas pathway mediates vasodilation and anti-inflammatory cascades. Activation of the ACE2/Ang-(1-7)/mas receptor axis has counteracting effects against the ACE/angiotensin II/AT1R pathway, activation of which contributes to hypertension, cardiac hypertrophy, heart failure, and other cardiovascular diseases. Therefore, ACE2 protects against RAAS-mediated pathologies by: (1) degrading Ang II to limit substrate availability in the adverse ACE/Ang II/AT1 receptor axis, and (2) generating Ang-(1-7) to increase substrate availability in the protective ACE2/Ang-(1-7)/mas receptor axis (Wang et al., Circulation 2020; March 26 (Epub ahead of print)).

ACE2 plays a very similar role in modulating the bradykinin pathway whereby des-arg-bradykinin binds and activates the inflammatory B1 receptor (McLean P G et al., J Exp Med 192 (3): 367-80) that is induced in tissue injury and ACE2 modulates the processing of des-arg⁹-bradykinin (Sodhi et al., Am J Physiol, 314: L17-L31, 2018) with likely binding of des-arg⁹-bradykinin breakdown products to the constitutively expressed anti-inflammatory B2 receptor.

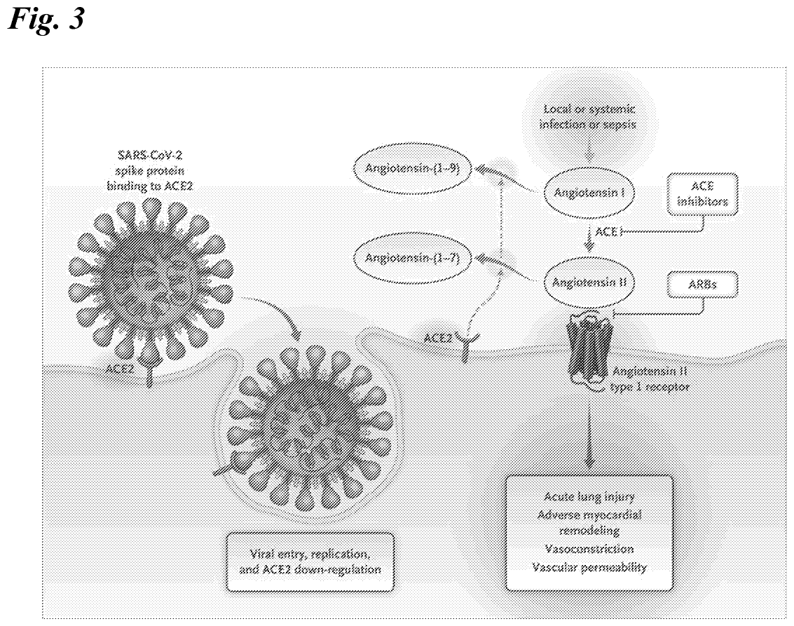

ACE2 is a functional receptor for certain types of viruses, especially coronaviruses, such as SARS-CoV-1 and SARS-CoV-2 (Moore et al., Nature 2003; 426:450-453). The trimeric spike glycoprotein (S protein) on the surface of the coronavirus binds primarily to the cellular receptor ACE2 on the surface of the host cell. The SARS-CoV-S protein is then primed by cellular surface proteases, such as transmembrane protease serine 2 (TMPRSS2), resulting in fusion of viral and cellular membranes and SARS-CoV entry and replication in host cells (See, FIG. 3 and Hoffman et al., Cell 2020; 181:271-280 and Li et al., eLife 2019; 8:e51230). As an enzyme, ACE2 is not normally consumed as it performs its many functions. In the context of binding to ACE2 as a receptor to gain cell entry, ACE2 is internalized upon binding to SARS-CoV-1 or SARS-CoV-2 (Zhang H., Intensive Care Med, 2020; Kuba K., Nature Medicine, 2005; Imai Y., Nature, 2005), thereby decreasing ACE2 expression on the cell-surface, likely below critical levels needed to maintain anti-inflammatory effect. Acquired ACE2 deficiency greatly reduces viral infection and replication in mice after experimental SARS-CoV infection (Kuba et al., Nat Med 2005; 11:875-879), suggesting that binding of the SARS-CoV-S protein to ACE2 is crucial for SARS-CoV infection. Examples of viruses that bind and enter host cells through the ACE2 receptor include, but are not limited to, SARS-CoV-1, SARS-CoV-2, and NL63/HCoV-NL63.

ACE2 is ubiquitously expressed on the cell-surface and can be shed from cells through proteolytic cleavage (Jia et al., Am J Physiol Lung Cell Mol Physiol, 2009, 297(1):L84-L96). ACE2 mRNA is detected in virtually all organs in humans and thus infection by SARS-CoV-2, for example, would be expected to cause systemic disease. Affected tissues include, but are not limited to, the oral mucosa, nasal mucosa, nasopharynx, heart, kidney, stomach, small intestine, colon, skin, lymph nodes, thymus, bone marrow, spleen, liver, brain, vasculature, and the lungs. In the lung, ACE2 expression is concentrated mainly in type II alveolar cells and macrophages and modestly in bronchial and tracheal epithelial cells (Hamming et al., J Pathol 2004; 203:631-7).

ACE2 regulates biological processes that may include angiotensin catabolism processes in blood, angiotensin maturation processes, angiotensin-mediated drinking behavior processes, positive regulation of cardiac muscle contraction processes, positive regulation of gap junction assembly processes, positive regulation of reactive oxygen species metabolism processes, receptor biosynthesis processes, receptor-mediated virion attachment processes (e.g., coronaviruses), regulation of cardiac conduction processes, regulation of cell proliferation processes, regulation of cytokine production processes, regulation of inflammatory response processes, regulation of systemic arterial blood pressure by renin-angiotensin processes, regulation of vasoconstriction processes, regulation of vasodilation processes, tryptophan transport processes, and viral entry into host cell processes (e.g., coronaviruses). The ACE2-Fc fusion proteins described herein may alter one or more of these biological processes. See, Batlle and Wysocki, U.S. Pub. No. US2018/0230447.

### Human ACE2 Sequences and Fragments and Variants Thereof.

The nucleotide sequence of the human ACE2 gene is available from the National Center for Biotechnology Information of the National Institutes of Health. The location of the human ACE2 gene is provided as NC 000023.11 (15494525 . . . 15602069, complement).

Human ACE2, isoform 1, is a transmembrane protein which is expressed first as a precursor polypeptide having the amino acid sequence of SEQ ID NO: 3. The human ACE2 protein comprises a signal peptide (amino acids 1-17), an extracellular domain (18-740), a helical transmembrane domain (741-761), and a cytoplasmic domain (762-805). All references to amino acid positions of the ACE2 protein are made in reference to SEQ ID NO: 3.

ACE2 naturally forms dimers which then bind ACE2 ligands with increased affinity and some degree of avidity to trigger additional biological functions (Yan et al 2020 Science 367 1444-1448). In some embodiments, the ACE2-Fc fusion protein comprises a natural ACE2 dimerization domain that is associated with dimerization of the ACE2-Fc fusion protein. In some embodiments, compositions comprising the ACE2-Fc fusion proteins described herein comprise about 20%, about 30%, about 40%, about 50%, about 60%, about 70%, about 80%, about 90%, or more of homodimers.

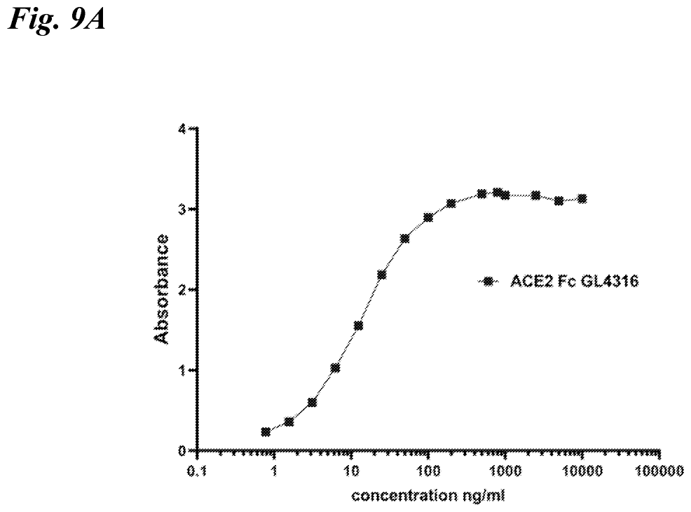

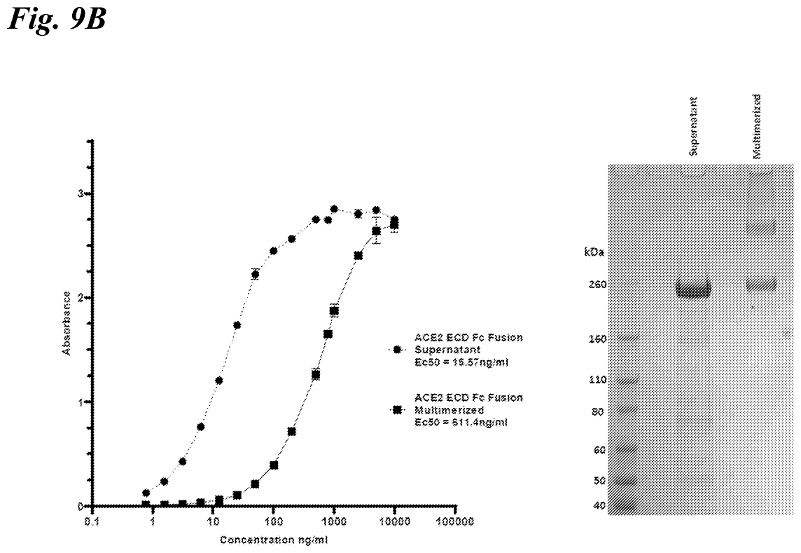

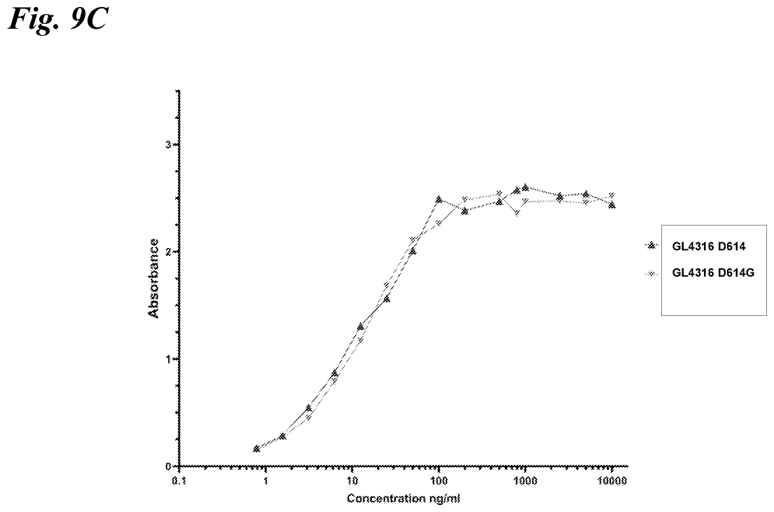

It is known in the art that ACE2 contains a dimerization domain (Clin Sci (Lond) (2020) 134 (23): 3229-3232, R, Yan et al., Science 367, 1444-1448 (2020)). It has further been reported that additional increases in apparent affinity can be achieved through inclusion of the ACE2 dimerization domain for avid binding and protein stabilization (W. Jing and E. Procko, Authorea November 2020). FIG. 9B demonstrates the unexpected and surprising finding that when ACE2-Fc fusion protein multimerizes (e.g. forms dimers of the homodimer, higher-order multimers, or aggregates) the ability of ACE2 to bind viral spike protein is diminished. In some embodiments, compositions comprising the ACE2-Fc fusion proteins described herein comprise a small amount (e.g., less than 20%) of dimers of the homodimer, higher-order multimers, or aggregates. In some embodiments, the present disclosure provides compositions comprising a plurality of the ACE2-Fc fusion proteins described herein and comprise no more than 20% w/w, 15% w/w, 10% w/w, 5% w/w, 4% w/w, 3% w/w, 2% w/w, 1% w/w, 0.9% w/w, 0.8% w/w, 0.7% w/w, 0.6% w/w, 0.5% w/w, 0.4% w/w, 0.3% w/w, 0.2% w/w, or 0.1% w/w of dimers of the ACE2-Fc fusion protein homodimer, higher-order multimers, or aggregates thereof. Unless otherwise specified, all references to a % protein in a particular composition are provided in weight/weight (w/w) units of measure determined based on optical density from SDS-PAGE or, more preferably, from UV spectroscopy from SEC HPLC. Herein, “higher order multimers” refer to multimers comprising 3, 4, 5, 6, or more homodimeric ACE2-Fc fusion proteins. “Aggregates” refer to unordered protein aggregates of the ACE2-Fc fusion proteins.

In some embodiments, the ACE2-Fc fusion protein comprises one or more mutations that decreases the formation of dimers of the homodimer, higher-order multimers, or aggregates. In some embodiments, the ACE2-Fc fusion protein comprises one or more mutations that decreases the formation of dimers of the homodimer, higher-order multimers, or aggregates by about 5%, about 10%, about 15%, about 20%, or about 25% compared to the parental ACE2-Fc fusion protein.

In some embodiments, the present disclosure provides compositions comprising a plurality of the ACE2-Fc fusion proteins described herein and comprise at least 80% ACE2-Fc fusion protein homodimers. In some embodiments, the compositions comprising the ACE2-Fc fusion proteins described herein comprise at least 85% w/w, at least 90% w/w, at least 95% w/w, at least 96% w/w, at least 97% w/w, at least 98% w/w, or at least 99% w/w ACE2-Fc fusion protein homodimers. In some embodiments, the compositions comprising the ACE2-Fc fusion proteins described herein comprise at least 99.1% w/w, at least 99.2% w/w, at least 99.3% w/w, at least 99.4% w/w, at least 99.5% w/w, at least 99.6% w/w, at least 99.7% w/w, at least 99.8% w/w, or at least 99.9% w/w ACE2-Fc fusion protein homodimers.

The large number of spike proteins expressed on the surface of coronaviruses can interact with multiple ACE2 receptors of a host cell that are in close proximity. The skilled artisan will surmise that multiple simultaneous binding events in close proximity between viral spike protein and host cell-bound ACE2 may lead to dimerization of ACE2 and that such dimerization is likely to trigger host cell signaling (See, Chen et al. J. Virol; August 2010, p. 7703-7712). Thus, in some embodiments, the ACE2-Fc fusion protein will bind a viral spike protein and prevent the viral spike protein from binding to host-cell surface ACE2, thus inhibiting ACE2 dimerization and cell signaling. In some embodiments, the ACE2-Fc fusion protein is optimized to decrease spike protein binding to host-cell surface ACE2 and thus prevent ACE2 dimerization and signaling. In some embodiments, the ACE2-Fc fusion protein is optimized to decrease spike protein binding to host-cell surface receptors other than ACE2, including but not limited to CD147 and NRP1, or to decrease binding to host cell membranes in the absence of receptor binding, in each case diminishing ACE2 dimerization and cell signaling.

In some embodiments, the ACE2-Fc fusion proteins described herein are optimized for use as a treatment for a disease or disorder, such as a non-infectious disease. A skilled artisan will appreciate that, under these circumstances, additional ACE2 functions may be beneficial. In some embodiments, the ACE2-Fc fusion protein is optimized to bind human ACE2 ligands, including but not limited to angiotensin (Ang) I, Ang II, apelin, pro-dynorphin, and des-arg⁹-bradykinin. In some embodiments, the optimized ACE2-Fc fusion protein binds human ACE2 ligands. In some embodiments, the optimized ACE2-Fc fusion protein forms homodimers. In some embodiments, the optimized ACE2-Fc fusion protein forms dimers of homodimers, higher-order multimers, and/or aggregates. In some embodiments, the optimized ACE2-Fc fusion protein comprises an ACE2 extracellular domain or fragment thereof that is at least 95%, 96%, 97%, 98%, 99% or 100% identical to an amino acid sequence selected from SEQ ID NOs: 8-38 and 51.

In some embodiments, the ACE2 Fc fusion protein described herein comprise two single-chain polypeptides or monomers, each comprising one or more Fc domain monomers and wherein at least one monomer comprises an ACE2 extracellular domain or fragment thereof. In some embodiments, the ACE2 Fc fusion protein described herein comprise two single-chain polypeptides or monomers, each comprising an ACE2 extracellular domain or fragment thereof and one or more Fc domain monomers. The association of the two ACE2-Fc fusion protein monomers forms ACE2-Fc fusion protein dimers or homodimers comprising at least one functional Fc domain and at least one ACE2 extracellular domain or fragment thereof and (See e.g., FIG. 1A and FIG. 1B). In particular embodiments, the ACE2-Fc fusion protein comprises at least one functional Fc domain and two ACE2 extracellular domains or fragments thereof.

In some embodiments, the ACE2 extracellular domains are variants of human ACE2. The disclosed ACE2 variants may comprise one or more amino acid mutations, deletions, additions or substitutions compared to the naturally occurring human ACE2 protein. Such amino acid modification may comprise introduction of modified or non-natural amino acids (nnAA). For example, the ACE2 variants of the present disclosure comprise one or more point mutations, or amino acid substitutions, in the extracellular domain of human ACE2.

The disclosed ACE2 variants may be modified to replace a natural amino acid residue by an nnAA. nnAAs may include, but are not limited to, an amino acid having a D-configuration, an N-methyl-α-amino acid, a non-proteogenic constrained amino acid, or a (3-amino acid.

Fragments of human ACE2 are also contemplated herein. Fragments of human ACE2 have been previously shown to dramatically increase cell and tissue penetration compared to full-length ACE2 (Wysocki et al., Biomolecules, 17; 9(12):886). As mentioned above, the extracellular domain of human ACE2 comprises amino acid residues 18-740 of SEQ ID NO: 3 following cleavage of the 17 amino acid signal peptide (SEQ ID NO: 2). In particular embodiments, the fragments of ACE2 are ligand-binding fragments. Any of the ACE2 fragments disclosed herein may be a ligand-binding fragment. In some embodiments, the ACE2-Fc fusion protein comprises an ACE2 extracellular domain or fragment thereof comprising amino acids 18-615 (SEQ ID NO: 8) of the ACE2 extracellular domain. In some embodiments, the ACE2-Fc fusion protein comprising a fragment of the ACE2 extracellular domain (e.g., SEQ ID NO: 8) exhibits enhanced cell and tissue penetration relative to an ACE2-Fc fusion protein comprising a full length ACE2 extracellular domain (e.g., SEQ ID NO: 6). In some embodiments, the ACE2-Fc fusion protein comprising a fragment of the ACE2 extracellular domain (e.g., SEQ ID NO: 8) retains the ability to bind to viral spike protein. In some embodiments, the ACE2-Fc fusion protein comprising a fragment of the ACE2 extracellular domain (e.g., SEQ ID NO: 8) retains the ability to bind human angiotensin II and/or other ACE2 ligands, such as des-arg⁹-bradykinin.

In some embodiments, the disclosed ACE2 variants are modified and the modification is selected from the group consisting of acylation, acetylation, formylation, lipolylation, myristoylation, palmitoylation, alkylation, isoprenylation, prenylation, and amidation. The modifications may be present at the N-terminus and/or C-terminus of the polypeptides (e.g., N-terminal acylation or acetylation, and/or C-terminal amidation). Modifications in the ACE2 polypeptide sequence may enhance the stability of the polypeptides, make the polypeptides resistant to proteolysis, or modulate functionality.

Table 2 provides amino acid sequences of human ACE2, including fragments and variants thereof. Signal peptide sequences are underlined and point mutations are bolded.

TABLE2
Exemplary Human ACE2 Sequences

| Name | Amino Acid Sequence | SEQ ID NO: |
|---|---|---|
| HuACE2, | MSSSSWLLLSLVAVTAAQSTIEEQAKTFLDKFNHEAEDLFYQSSLASWNYNTNIT | 3 |
| full length, | EENVQNMNNAGDKWSAFLKEQSTLAQMYPLQEIQNLTVKLQLQALQQNGSSVLSE | |
| isoform 1 | DKSKRLNTILNTMSTIYSTGKVCNPDNPQECLLLEPGLNEIMANSLDYNERLWAW | |
| | ESWRSEVGKQLRPLYEEYVVLKNEMARANHYEDYGDYWRGDYEVNGVDGYDYSRG | |
| | QLIEDVEHTFEEIKPLYEHLHAYVRAKLMNAYPSYISPIGCLPAHLLGDMWGRFW | |
| | TNLYSLTVPFGQKPNIDVTDAMVDQAWDAQRIFKEAEKFFVSVGLPNMTQGFWEN | |
| | SMLTDPGNVQKAVCHPTAWDLGKGDFRILMCTKVTMDDFLTAHHEMGHIQYDMAY | |
| | AAQPFLLRNGANEGFHEAVGEIMSLSAATPKHLKSIGLLSPDFQEDNETEINFLL | |
| | KQALTIVGTLPFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLHKCDISNSTEA | |
| | GQKLFNMLRLGKSEPWTLALENVVGAKNMNVRPLLNYFEPLFTWLKDQNKNSFVG | |
| | WSTDWSPYADQSIKVRISLKSALGDKAYEWNDNEMYLFRSSVAYAMRQYFLKVKN | |
| | QMILFGEEDVRVANLKPRISFNFFVTAPKNVSDIIPRTEVEKAIRMSRSRINDAF | |
| | RLNDNSLEFLGIQPTLGPPNQPPVSIWLIVFGVVMGVIVVGIVILIFTGIRDRKK | |
| | KNKARSGENPYASIDISKGENNPGFQNTDDVQTSF | |
| HuACE2, | MSSSSWLLLSLVAVTAAQSTIEEQAKTFLDKFNHEAEDLFYQSSLASWNYNTNIT | 4 |
| full length, | EENVQNMNNAGDKWSAFLKEQSTLAQMYPLQEIQNLTVKLQLQALQQNGSSVLSE | |
| isoform 2 | DKSKRLNTILNTMSTIYSTGKVCNPDNPQECLLLEPGLNEIMANSLDYNERLWAW | |
| | ESWRSEVGKQLRPLYEEYVVLKNEMARANHYEDYGDYWRGDYEVNGVDGYDYSRG | |
| | QLIEDVEHTFEEIKPLYEHLHAYVRAKLMNAYPSYISPIGCLPAHLLGDMWGRFW | |
| | TNLYSLTVPFGQKPNIDVTDAMVDQAWDAQRIFKEAEKFFVSVGLPNMTQGFWEN | |
| | SMLTDPGNVQKAVCHPTAWDLGKGDFRILMCTKVTMDDFLTAHHEMGHIQYDMAY | |
| | AAQPFLLRNGANEGFHEAVGEIMSLSAATPKHLKSIGLLSPDFQEDNETEINFLL | |
| | KQALTIVGTLPFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLHKCDISNSTEA | |
| | GQKLL | |
| HuACE2- | MSSSSWLLLSLVAVTAAQSTIEEQAKTFLDKFNHEAEDLFYQSSLASWNYNTNIT | 5 |
| SP-ECD | EENVQNMNNAGDKWSAFLKEQSTLAQMYPLQEIQNLTVKLQLQALQQNGSSVLSE | |
| (native SP) | DKSKRLNTILNTMSTIYSTGKVCNPDNPQECLLLEPGLNEIMANSLDYNERLWAW | |
| | ESWRSEVGKQLRPLYEEYVVLKNEMARANHYEDYGDYWRGDYEVNGVDGYDYSRG | |
| | QLIEDVEHTFEEIKPLYEHLHAYVRAKLMNAYPSYISPIGCLPAHLLGDMWGRFW | |
| | TNLYSLTVPFGQKPNIDVTDAMVDQAWDAQRIFKEAEKFFVSVGLPNMTQGFWEN | |
| | SMLTDPGNVQKAVCHPTAWDLGKGDFRILMCTKVTMDDFLTAHHEMGHIQYDMAY | |
| | AAQPFLLRNGANEGFHEAVGEIMSLSAATPKHLKSIGLLSPDFQEDNETEINFLL | |
| | KQALTIVGTLPFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLHKCDISNSTEA | |
| | GQKLFNMLRLGKSEPWTLALENVVGAKNMNVRPLLNYFEPLFTWLKDQNKNSFVG | |
| | WSTDWSPYADQSIKVRISLKSALGDKAYEWNDNEMYLFRSSVAYAMRQYFLKVKN | |
| | QMILFGEEDVRVANLKPRISFNFFVTAPKNVSDIIPRTEVEKAIRMSRSRINDAF | |
| | RLNDNSLEFLGIQPTLGPPNQPPVS | |
| HuACE2- | QSTIEEQAKTFLDKFNHEAEDLFYQSSLASWNYNTNITEENVQNMNNAGDKWSAF | 6 |
| ECD | LKEQSTLAQMYPLQEIQNLTVKLQLQALQQNGSSVLSEDKSKRLNTILNTMSTIY | |
| | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYADQSIKVRI | |
| | SLKSALGDKAYEWNDNEMYLFRSSVAYAMRQYFLKVKNQMILFGEEDVRVANLKP | |
| | RISFNFFVTAPKNVSDIIPRTEVEKAIRMSRSRINDAFRLNDNSLEFLGIQPTLG | |
| | PPNQPPVS | |
| HuACE2- | MSSSSWLLLSLVAVTAAQSTIEEQAKTFLDKFNHEAEDLFYQSSLASWNYNTNIT | 7 |
| SP-ECD615 | EENVQNMNNAGDKWSAFLKEQSTLAQMYPLQEIQNLTVKLQLQALQQNGSSVLSE | |
| (native SP) | DKSKRLNTILNTMSTIYSTGKVCNPDNPQECLLLEPGLNEIMANSLDYNERLWAW | |
| | ESWRSEVGKQLRPLYEEYVVLKNEMARANHYEDYGDYWRGDYEVNGVDGYDYSRG | |
| | QLIEDVEHTFEEIKPLYEHLHAYVRAKLMNAYPSYISPIGCLPAHLLGDMWGRFW | |
| | TNLYSLTVPFGQKPNIDVTDAMVDQAWDAQRIFKEAEKFFVSVGLPNMTQGFWEN | |
| | SMLTDPGNVQKAVCHPTAWDLGKGDFRILMCTKVTMDDFLTAHHEMGHIQYDMAY | |
| | AAQPFLLRNGANEGFHEAVGEIMSLSAATPKHLKSIGLLSPDFQEDNETEINFLL | |
| | KQALTIVGTLPFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLHKCDISNSTEA | |
| | GQKLFNMLRLGKSEPWTLALENVVGAKNMNVRPLLNYFEPLFTWLKDQNKNSFVG | |
| | WSTDWSPYAD | |
| HuACE2- | QSTIEEQAKTFLDKFNHEAEDLFYQSSLASWNYNTNITEENVQNMNNAGDKWSAF | 8 |
| ECD615 | LKEQSTLAQMYPLQEIQNLTVKLQLQALQQNGSSVLSEDKSKRLNTILNTMSTIY | |
| | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 9 |
| ECD615 | LKEQSTLAQNYPLQEIQNLTVKLQLQALQQNGSSVLSEDKSKRLNTILNTMSTIY | |
| (M82N) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 10 |
| ECD615 | LKEQSTLAQAYPLQEIQNLTVKLQLQALQQNGSSVLSEDKSKRLNTILNTMSTIY | |
| (M82A) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 11 |
| ECD615 | LKEQSTLAQDYPLQEIQNLTVKLQLQALQQNGSSVLSEDKSKRLNTILNTMSTIY | |
| (M82D) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 12 |
| ECD615 | LKEQSTLAQSYPLQEIQNLTVKLQLQALQQNGSSVLSEDKSKRLNTILNTMSTIY | |
| (M82S) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLEKFNHEAEDLFYQSSLASWNYNTNITEENVQNMNNAGDKWSAF | 13 |
| ECD615 | LKEQSTLAQMYPLQEIQNLTVKLQLQALQQNGSSVLSEDKSKRLNTILNTMSTIY | |
| (D30E) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDTFNHEAEDLFYQSSLASWNYNTNITEENVQNMNNAGDKWSAF | 14 |
| ECD615 | LKEQSTLAQMYPLQEIQNLTVKLQLQALQQNGSSVLSEDKSKRLNTILNTMSTIY | |
| (K31T) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNQEAEDLFYQSSLASWNYNTNITEENVQNMNNAGDKWSAF | 15 |
| ECD615 | LKEQSTLAQMYPLQEIQNLTVKLQLQALQQNGSSVLSEDKSKRLNTILNTMSTIY | |
| (H34Q) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEELFYQSSLASWNYNTNITEENVQNMNNAGDKWSAF | 16 |
| ECD615 | LKEQSTLAQMYPLQEIQNLTVKLQLQALQQNGSSVLSEDKSKRLNTILNTMSTIY | |
| (D38E) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 17 |
| ECD615 | LKEQSTLAQMYPLQEIQNLTVKLQLQALQQNGSSVLSEDKSKRLNTILNTMSTIY | |
| (Q139S) | STGKVCNPDNPSECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 18 |
| ECD615 | LKEQSTLAQMYPLQEIQNLTVKLQLQALQQNGSSVLSEDKSKRLNTILNTMSTIY | |
| (Q139A) | STGKVCNPDNPAECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 19 |
| ECD615 | LKEQSTLAQMYPLQEIQNLTVKLQLQALQQNGSSVLSEDKSKRLNTILNTMSTIY | |
| (Q139V) | STGKVCNPDNPVECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 20 |
| ECD615 | LKEQSTLAQMYPLQEIQNLTVKLQLQALQQNGSSVLSEDKSKRLNTILNTMSTIY | |
| (Q175S) | STGKVCNPDNPQECLLLEPGLNEIMANSLDYNERLWAWESWRSEVGKSLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 21 |
| ECD615 | LKEQSTLAQMYPLQEIQNLTVKLQLQALQQNGSSVLSEDKSKRLNTILNTMSTIY | |
| (Q175A) | STGKVCNPDNPQECLLLEPGLNEIMANSLDYNERLWAWESWRSEVGKALRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 22 |
| ECD615 | LKEQSTLAQMYPLQEIQNLTVKLQLQALQQNGSSVLSEDKSKRLNTILNTMSTIY | |
| (Q175V) | STGKVCNPDNPQECLLLEPGLNEIMANSLDYNERLWAWESWRSEVGKVLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 23 |
| ECD615 | LKEQSTLAQMYPLQEIQNLTVKLQLQALQQNGSSVLSEDKSKRLNTILNTMSTIY | |
| (H374S) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHSEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 24 |
| ECD615 | LKEQSTLAQMYPLQEIQNLTVKLQLQALQQNGSSVLSEDKSKRLNTILNTMSTIY | |
| (H374A) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHAEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 25 |
| ECD615 | LKEQSTLAQMYPLQEIQNLTVKLQLQALQQNGSSVLSEDKSKRLNTILNTMSTIY | |
| (H374V) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHVEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 26 |
| ECD615 | LKEQSTLAQMYPLQEIQNLTVKLQLQALQQNGSSVLSEDKSKRLNTILNTMSTIY | |
| (H378S) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGSIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 27 |
| ECD615 | LKEQSTLAQMYPLQEIQNLTVKLQLQALQQNGSSVLSEDKSKRLNTILNTMSTIY | |
| (H378A) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGAIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 28 |
| ECD615 | LKEQSTLAQMYPLQEIQNLTVKLQLQALQQNGSSVLSEDKSKRLNTILNTMSTIY | |
| (H378V) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGVIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEELFYQSSLASWNYNTNITEENVQNMNNAGDKWSAF | 29 |
| ECD615 | LKEQSTLAQNYPLQEIQNLTVKLQLQALQQNGSSVLSEDKSKRLNTILNTMSTIY | |
| (M82N/D38E) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEELFYQSSLASWNYNTNITEENVQNMNNAGDKWSAF | 30 |
| ECD615 | LKEQSTLAQAYPLQEIQNLTVKLQLQALQQNGSSVLSEDKSKRLNTILNTMSTIY | |
| (M82A/D38E) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLEKFNHEAEDLFYQSSLASWNYNTNITEENVQNMNNAGDKWSAF | 31 |
| ECD615 | LKEQSTLAQTYPLQEIQNLTVKLQLQALQQNGSSVLSEDKSKRLNTILNTMSTIY | |
| (M82T/D30E) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 32 |
| ECD615 | LKEQSTLAQNYPLQEIQNLTVKLQLQALQQNGSSVLSEDKSKRLNTILNTMSTIY | |
| (M82N/Q139A) | STGKVCNPDNPAECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 33 |
| ECD615 | LKEQSTLAQNYPLQEIQNLTVKLQLQALQQNGSSVLSEDKSKRLNTILNTMSTIY | |
| (M82N/Q175A) | STGKVCNPDNPQECLLLEPGLNEIMANSLDYNERLWAWESWRSEVGKALRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 34 |
| ECD615 | LKEQSTLAQNYPLQEIQNLTVKLQLQALQQNGSSVLSEDKSKRLNTILNTMSTIY | |
| (M82N/H374S) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHSEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 35 |
| ECD615 | LKEQSTLAQNYPLQEIQNLTVKLQLQALQQNGSSVLSEDKSKRLNTILNTMSTIY | |
| (M82N/H378S) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGSIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLQKFNHKAEDLFYQSSLASWNYNTNITEENVQNMNNAGDKWSAF | 36 |
| ECD615 | LKEQSTLAQMYPLQEIQNLTVKLQLQALQQNGSSVLSEDKSKRLNTILNTMSTIY | |
| (D30Q/E35K) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 37 |
| ECD615 | LKEQSTLAQNYPLQEIQNLTVKLQLQALQQNGSSVLSEDKSKRLNTILNTMSTIY | |
| (M82N/Q175A/ | STGKVCNPDNPQECLLLEPGLNEIMANSLDYNERLWAWESWRSEVGKALRPLYEE | |
| H374S/H378S) | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHSEMGSIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFYQSSLASWNYNTNITEENVQNMNNAGDKWSAF | 38 |
| ECD615 | LKEQSTLAQNYPLQEIQNLTVKLQLQALQQNGSSVLSEDKSKRLNTILNTMSTIY | |
| (M82N/Q139A/ | STGKVCNPDNPAECLLLEPGLNEIMANSLDYNERLWAWESWRSEVGKQLRPLYEE | |
| H374S/ | YVVLKNEMARANHYEDYGDYWRGDYEVNGVDGYDYSRGQLIEDVEHTFEEIKPLY | |
| H378S) | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHSEMGSIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |
| HuACE2- | QSTIEEQAKTFLDKFNHEAEDLFHESSLASWNYNTNITEENVQNMNNAGDKWSAF | 51 |
| ECD615 | LKEQSTLAQMYPLQEIQNLTVKLQLQALQQNGSSVLSEDKSKRLNTILNTMSTIY | |
| (Y41H/Q42E) | STGKVCNPDNPQECLLLEPGLNEIMANSLDYNERLWAWESWRSEVGKQLRPLYEE | |
| | YVVLKNEMARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPFGQKPNID | |
| | VTDAMVDQAWDAQRIFKEAEKFFVSVGLPNMTQGFWENSMLTDPGNVQKAVCHPT | |
| | AWDLGKGDFRILMCTKVTMDDFLTAHHEMGHIQYDMAYAAQPFLLRNGANEGFHE | |
| | AVGEIMSLSAATPKHLKSIGLLSPDFQEDNETEINFLLKQALTIVGTLPFTYMLE | |
| | KWRWMVFKGEIPKDQWMKKWWEMKREIVGVVEPVPHDETYCDPASLFHVSNDYSF | |
| | IRYYTRTLYQFQFQEALCQAAKHEGPLHKCDISNSTEAGQKLFNMLRLGKSEPWT | |
| | LALENVVGAKNMNVRPLLNYFEPLFTWLKDQNKNSFVGWSTDWSPYAD | |

The fragment of the ACE2 extracellular domain may be a ligand-binding fragment. In some embodiments, the ligand-binding fragment may bind to alphacoronavirus or a betacoronavirus. In some embodiments, the ACE2 extracellular domain or ligand-binding fragment thereof may bind to alphacoronavirus or a betacoronavirus.

In some embodiments, the ACE2 extracellular domain or ligand-binding fragment thereof specifically binds to an alphacoronavirus (e.g., HCoV-NL63). In some embodiments, the ACE2 extracellular domain or ligand-binding fragment thereof specifically binds to a betacoronavirus (e.g., SARS-CoV-1, SARS-CoV-2). In some embodiments, the ACE2 extracellular domain or ligand-binding fragment thereof specifically binds to SARS-CoV-1. In some embodiments, the ACE2 extracellular domain or ligand-binding fragment thereof specifically binds to SARS-CoV-2. In some embodiments, the ACE2 extracellular domain or ligand-binding fragment thereof specifically binds to viral spike protein, e.g., SARS-CoV-2 spike protein. In some embodiments, the ACE2 extracellular domain or ligand-binding fragment thereof specifically binds to angiotensin II. In some embodiments, the ACE2 extracellular domain or ligand-binding fragment thereof cleaves angiotensin II to generate angiotensin-(1-7). In a particular embodiment, the ACE2 extracellular domain or ligand-binding fragment thereof specifically binds to a SARS-CoV-2 spike protein.

In some embodiments, the ACE2 extracellular domain or ligand-binding fragment thereof comprises a signal peptide on the N-terminus. In some embodiments, the ACE2 extracellular domain or ligand-binding fragment thereof comprises one or more Fc domains on the C-terminus.

In some embodiments, the ACE2 extracellular domain or fragment thereof specifically binds to an alphacoronavirus (e.g., HCoV-NL63) and comprises an amino acid sequence that is at least 80%, 85%, 90%, 95%, 96%, 97%, 98%, or 99% identical to SEQ ID NO: 6. In some embodiments, the ACE2 extracellular domain or fragment thereof specifically binds to a betacoronavirus (e.g., SARS-CoV-1, SARS-CoV-2) and comprises an amino acid sequence that is at least 80%, 85%, 90%, 95%, 96%, 97%, 98%, or 99% identical to SEQ ID NO: 6. In some embodiments, the ACE2 extracellular domain or fragment thereof specifically binds to SARS-CoV-1 and comprises an amino acid sequence that is at least 80%, 85%, 90%, 95%, 96%, 97%, 98%, or 99% identical to SEQ ID NO: 6. In some embodiments, the ACE2 extracellular domain or fragment thereof specifically binds to SARS-CoV-2 and comprises an amino acid sequence that is at least 80%, 85%, 90%, 95%, 96%, 97%, 98%, or 99% identical to SEQ ID NO: 6. In some embodiments, the ACE2 extracellular domain or fragment thereof specifically binds to viral spike protein, e.g., SARS-CoV-2 spike protein, and comprises an amino acid sequence that is at least 80%, 85%, 90%, 95%, 96%, 97%, 98%, or 99% identical to SEQ ID NO: 6. In some embodiments, the ACE2 extracellular domain or fragment thereof specifically binds to angiotensin II, and comprises an amino acid sequence that is at least 80%, 85%, 90%, 95%, 96%, 97%, 98%, or 99% identical to SEQ ID NO: 6. In some embodiments, the ACE2 extracellular domain or fragment thereof cleaves angiotensin II to generate angiotensin-(1-7), and comprises an amino acid sequence that is at least 80%, 85%, 90%, 95%, 96%, 97%, 98%, or 99% identical to SEQ ID NO: 6. In some embodiments, the ACE2 extracellular domain or fragment thereof comprises a signal peptide on the N-terminus. In some embodiments, the signal peptide comprises the amino acid sequence of SEQ ID NO: 1. In other embodiments, the signal peptide comprises the amino acid sequence of SEQ ID NO: 2. In some embodiments, the ACE2 extracellular domain or fragment thereof comprises one or more Fc domains on the C-terminus. In some embodiments, the one or more Fc domains comprises an amino acid sequence selected from the group consisting of SEQ ID NOs: 39-42.

In some embodiments, the ACE2 extracellular domain fragment specifically binds to an alphacoronavirus (e.g., 229E, NL62, OC43, HKU1) and comprises an amino acid sequence that is at least 80%, 85%, 90%, 95%, 96%, 97%, 98%, or 99% identical to SEQ ID NO: 8. In some embodiments, the ACE2 extracellular domain fragment specifically binds to a betacoronavirus (e.g., MERS-CoV, SARS-CoV-1, SARS-CoV-2) and comprises an amino acid sequence that is at least 80%, 85%, 90%, 95%, 96%, 97%, 98%, or 99% identical to SEQ ID NO: 8. In some embodiments, the ACE2 extracellular domain fragment specifically binds to SARS-CoV-1 and comprises an amino acid sequence that is at least 80%, 85%, 90%, 95%, 96%, 97%, 98%, or 99% identical to SEQ ID NO: 8. In some embodiments, the ACE2 extracellular domain fragment specifically binds to SARS-CoV-2 and comprises an amino acid sequence that is at least 80%, 85%, 90%, 95%, 96%, 97%, 98%, or 99% identical to SEQ ID NO: 8. In some embodiments, the ACE2 extracellular domain fragment specifically binds to viral spike protein, e.g., SARS-CoV-2 spike protein, and comprises an amino acid sequence that is at least 80%, 85%, 90%, 95%, 96%, 97%, 98%, or 99% identical to SEQ ID NO: 8. In some embodiments, the ACE2 extracellular domain fragment specifically binds to angiotensin II, and comprises an amino acid sequence that is at least 80%, 85%, 90%, 95%, 96%, 97%, 98%, or 99% identical to SEQ ID NO: 8. In some embodiments, the ACE2 extracellular domain or fragment thereof cleaves angiotensin II to generate angiotensin-(1-7), and comprises an amino acid sequence that is at least 80%, 85%, 90%, 95%, 96%, 97%, 98%, or 99% identical to SEQ ID NO: 8. In some embodiments, the ACE2 extracellular domain or fragment thereof binds viral spike protein and has diminished ability to cleave angiotensin II to generate angiotensin-(1-7), and comprises an amino acid sequence that is at least 80%, 85%, 90%, 95%, 96%, 97%, 98%, or 99% identical to SEQ ID NO: 8. In some embodiments, the ACE2 extracellular domain or fragment thereof comprises a signal peptide on the N-terminus. In some embodiments, the signal peptide comprises the amino acid sequence of SEQ ID NO: 1. In other embodiments, the signal peptide comprises the amino acid sequence of SEQ ID NO: 2. In some embodiments, the ACE2 extracellular domain or fragment thereof comprises one or more Fc domains on the C-terminus. In some embodiments, the one or more Fc domains comprises an amino acid sequence selected from the group consisting of SEQ ID NOs: 39-42. In some embodiments, the ACE2 extracellular domain or fragment thereof with at least one Fc domain on the C-terminus is an IgG4 Fc domain. In some embodiments, the IgG4 Fc domain comprises an amino acid sequence that is 95%, 96%, 97%, 98%, or 99% or greater identical to SEQ ID NO: 42.

In some embodiments, the ACE2 extracellular domain or fragment thereof comprises one or more point mutations. In some embodiments, the one or more point mutations in the ACE2 extracellular domain or fragment thereof increases binding to viral spike protein. In some embodiments, the one or more point mutations in the ACE2 extracellular domain or fragment thereof increases binding to the SARS-CoV-2 viral spike protein. In some embodiments, the one or more point mutations in the ACE2 extracellular domain or fragment thereof increases binding to SARS-CoV-2 viral spike protein that has evolved. In some embodiments, the evolved SARS-CoV-2 viral spike protein to which the ACE2 extracellular domain or fragment thereof binds comprises viral D614G. In some embodiments, the one or more point mutations in the ACE2 extracellular domain or fragment thereof decreases the formation of dimers of a homodimer, higher-order multimers, or aggregates. In some embodiments, the one or more point mutations in the ACE2 extracellular domain or fragment thereof decreases the binding to angiotensin II or decreases enzymatic activity when bound to angiotensin II. In some embodiments, the one or more point mutations are located at any of positions 24, 27, 28, 30, 31, 34, 35, 37, 38, 41, 42, 45, 82, 83, 139, 175, 330, 353, 354, 355, 357, 374, 378, and 393. In some embodiments, the one or more point mutations are located at any of positions 24, 27, 28, 30, 31, 34, 35, 37, 38, 41, 42, 45, 82, 83, 139, 175, 330, 353, 354, 355, 357, 374, 378, and 393 of SEQ ID NO: 5 or SEQ ID NO: 7.

In some embodiments, the ACE2 extracellular domain or fragment thereof comprises a point mutation at position 82. In some embodiments, the ACE2 extracellular domain or fragment thereof comprises a point mutation at position 82 of SEQ ID NO: 5 or SEQ ID NO: 7. In some embodiments, the ACE2 extracellular domain or fragment thereof comprises the M82N, M82A, M82D, M82S, M82T, M82K, or M82I point mutation. In some embodiments, the ACE2 extracellular domain or fragment thereof comprises the M82N point mutation. In some embodiments, the ACE2 extracellular domain or fragment thereof comprises the M82A point mutation. In some embodiments, the ACE2 extracellular domain or fragment thereof comprises the M82D point mutation. In some embodiments, the ACE2 extracellular domain or fragment thereof comprises the M82S point mutation. In some embodiments, a point mutation at position 82 of the ACE2 extracellular domain or fragment thereof increases binding to viral spike protein, e.g., SARS-CoV-2. In some embodiments, the point mutation M82N, M82A, M82D, or M82S in the ACE2 extracellular domain or fragment thereof increases binding to viral spike protein, e.g., SARS-CoV-2.

In some embodiments, the ACE2 extracellular domain or fragment thereof comprises a point mutation at position 30. In some embodiments, the ACE2 extracellular domain or fragment thereof comprises a point mutation at position 30 of SEQ ID NO: 5 or SEQ ID NO: 7. In some embodiments, the ACE2 extracellular domain or fragment thereof comprises the D30E, D30T, D30A, D30S, D30Q, or D30V point mutation. In some embodiments, the ACE2 extracellular domain or fragment thereof comprises the D30E point mutation. In some embodiments, the ACE2 extracellular domain or fragment thereof comprises the D30Q point mutation. In some embodiments, a point mutation at position 30 of the ACE2 extracellular domain or fragment thereof increases binding to viral spike protein, e.g., SARS-CoV-2. In some embodiments, the point mutation D30E or D30Q in the ACE2 extracellular domain or fragment thereof increases binding to viral spike protein, e.g., SARS-CoV-2.

In some embodiments, the ACE2 extracellular domain or fragment thereof comprises a point mutation at position 31. In some embodiments, the ACE2 extracellular domain or fragment thereof comprises a point mutation at position 31 of SEQ ID NO: 5 or SEQ ID NO: 7. In some embodiments, the ACE2 extracellular domain or fragment thereof comprises the K31T, K31D, K31E, K31N, or K31Q point mutation. In some embodiments, the ACE2 extracellular domain or fragment thereof comprises the K31T point mutation. In some embodiments, a point mutation at position 31 of the ACE2 extracellular domain or fragment thereof increases binding to viral spike protein, e.g., SARS-CoV-2. In some embodiments, the point mutation K31T in the ACE2 extracellular domain or fragment thereof increases binding to viral spike protein, e.g., SARS-CoV-2.

In some embodiments, the ACE2 extracellular domain or fragment thereof comprises a point mutation at position 34. In some embodiments, the ACE2 extracellular domain or fragment thereof comprises a point mutation at position 34 of SEQ ID NO: 5 or SEQ ID NO: 7. In some embodiments, the ACE2 extracellular domain or fragment thereof comprises the H34A, H34T, H34S, H34K, H34V, H34P, or H34R point mutation. In some embodiments, the ACE2 extracellular domain or fragment thereof comprises the H34Q point mutation. In some embodiments, a point mutation at position 34 of the ACE2 extracellular domain or fragment thereof increases binding to viral spike protein, e.g., SARS-CoV-2. In some embodiments, the point mutation H34Q in the ACE2 extracellular domain or fragment thereof increases binding to viral spike protein, e.g., SARS-CoV-2.

In some embodiments, the ACE2 extracellular domain or fragment thereof comprises a point mutation at position 35. In some embodiments, the ACE2 extracellular domain or fragment thereof comprises a point mutation at position 35 of SEQ ID NO: 5 or SEQ ID NO: 7. In some embodiments, the ACE2 extracellular domain or fragment thereof comprises the E35K or E35Q point mutation. In some embodiments, a point mutation at position 35 of the ACE2 extracellular domain or fragment thereof increases binding to viral spike protein, e.g., SARS-CoV-2. In some embodiments, the point mutation E35K or E35Q in the ACE2 extracellular domain or fragment thereof increases binding to viral spike protein, e.g., SARS-CoV-2.

In some embodiments, the ACE2 extracellular domain or fragment thereof comprises a point mutation at position 38. In some embodiments, the ACE2 extracellular domain or fragment thereof comprises a point mutation at position 38 of SEQ ID NO: 5 or SEQ ID NO: 7. In some embodiments, the ACE2 extracellular domain or fragment thereof comprises the D38E or D38N point mutation. In some embodiments, the ACE2 extracellular domain or fragment thereof comprises the D38E point mutation. In some embodiments, a point mutation at position 38 of the ACE2 extracellular domain or fragment thereof increases binding to viral spike protein, e.g., SARS-CoV-2. In some embodiments, the point mutation D38E in the ACE2 extracellular domain or fragment thereof increases binding to viral spike protein, e.g., SARS-CoV-2.

In some embodiments, the ACE2 extracellular domain or fragment thereof comprises a point mutation at position 139. In some embodiments, the ACE2 extracellular domain or fragment thereof comprises a point mutation at position 139 of SEQ ID NO: 5 or SEQ ID NO: 7. In some embodiments, the ACE2 extracellular domain or fragment thereof comprises the Q139A point mutation. In some embodiments, the ACE2 extracellular domain or fragment thereof comprises the Q139S point mutation. In some embodiments, the ACE2 extracellular domain or fragment thereof comprises the Q139V point mutation. In some embodiments, a point mutation at position 139 of the ACE2 extracellular domain or fragment thereof decreases the formation of dimers of a homodimer, higher-order multimers, or aggregates. In some embodiments, the point mutation Q139A, Q139S, or Q139V in the ACE2 extracellular domain or fragment thereof decreases the formation of dimers of a homodimer, higher-order multimers, or aggregates.

In some embodiments, the ACE2 extracellular domain or fragment thereof comprises a point mutation at position 175. In some embodiments, the ACE2 extracellular domain or fragment thereof comprises a point mutation at position 175 of SEQ ID NO: 5 or SEQ ID NO: 7. In some embodiments, the ACE2 extracellular domain or fragment thereof comprises the Q175A point mutation. In some embodiments, the ACE2 extracellular domain or fragment thereof comprises the Q175S point mutation. In some embodiments, the ACE2 extracellular domain or fragment thereof comprises the Q175V point mutation. In some embodiments, the point mutation Q175A, Q175S, or Q175V in the ACE2 extracellular domain or fragment thereof decreases the formation of dimers of a homodimer, higher-order multimers, or aggregates.

In some embodiments, the ACE2 extracellular domain or fragment thereof comprises a point mutation at position 353. In some embodiments, the ACE2 extracellular domain or fragment thereof comprises a point mutation at position 353 of SEQ ID NO: 5 or SEQ ID NO: 7. In some embodiments, the ACE2 extracellular domain or fragment thereof comprises the K353H, K353N, or K353R point mutation. In some embodiments, a point mutation at position 353 of the ACE2 extracellular domain or fragment thereof increases binding to viral spike protein, e.g., SARS-CoV-2. In some embodiments, the point mutation K353H, K353N, or K353R in the ACE2 extracellular domain or fragment thereof increases binding to viral spike protein, e.g., SARS-CoV-2.

In some embodiments, the ACE2 extracellular domain or fragment thereof comprises a point mutation at position 374. In some embodiments, the ACE2 extracellular domain or fragment thereof comprises a point mutation at position 374 of SEQ ID NO: 5 or SEQ ID NO: 7. In some embodiments, the ACE2 extracellular domain or fragment thereof comprises the H374S point mutation. In some embodiments, the ACE2 extracellular domain or fragment thereof comprises the H374A point mutation. In some embodiments, the ACE2 extracellular domain or fragment thereof comprises the H374V point mutation. In some embodiments, a point mutation at position 374 of the ACE2 extracellular domain or fragment thereof decreases binding to angiotensin II and/or decreases enzymatic activity when bound to angiotensin II. In some embodiments, the point mutation H374S, H374A, or H374V in the ACE2 extracellular domain or fragment thereof decreases binding to angiotensin II and/or decreases enzymatic activity when bound to angiotensin II.

In some embodiments, the ACE2 extracellular domain or fragment thereof comprises a point mutation at position 378. In some embodiments, the ACE2 extracellular domain or fragment thereof comprises a point mutation at position 378 of SEQ ID NO: 5 or SEQ ID NO: 7. In some embodiments, the ACE2 extracellular domain or fragment thereof comprises the H378S point mutation. In some embodiments, the ACE2 extracellular domain or fragment thereof comprises the H378A point mutation. In some embodiments, the ACE2 extracellular domain or fragment thereof comprises the H378V point mutation. In some embodiments, a point mutation at position 378 of the ACE2 extracellular domain or fragment thereof decreases binding to angiotensin II and/or decreases enzymatic activity when bound to angiotensin II. In some embodiments, the point mutation H378S, H378A, or H378V in the ACE2 extracellular domain or fragment thereof decreases binding to angiotensin II and/or decreases enzymatic activity when bound to angiotensin II.

In some embodiments, the ACE2 extracellular domain or fragment thereof comprises a point mutation at positions 41 and 42. In some embodiments, the ACE2 extracellular domain or fragment thereof comprises a point mutation at positions 41 and 42 of SEQ ID NO: 5 or SEQ ID NO: 7. In some embodiments, the ACE2 extracellular domain or fragment thereof comprises the Y41H and Q42E point mutations.

In some embodiments, the ACE2-Fc fusion proteins described herein comprise an ACE2 extracellular domain or fragment thereof and the ACE2 extracellular domain or fragment thereof comprises one or more point mutations. In some embodiments, the ACE2 extracellular domain or fragment thereof comprises one point mutation. In some embodiments, the ACE2 extracellular domain or fragment thereof comprises two point mutations. In some embodiments, the ACE2 extracellular domain or fragment thereof comprises three point mutations. In some embodiments, the ACE2 extracellular domain or fragment thereof comprises four point mutations. In some embodiments, the ACE2 extracellular domain or fragment thereof comprises five point mutations. In some embodiments, the ACE2 extracellular domain or fragment thereof comprises six, seven, eight, nine, or ten point mutations.

In some embodiments, the ACE2 extracellular domain or fragment thereof comprises two or more point mutations at any of positions 24, 27, 28, 30, 31, 34, 35, 37, 38, 41, 42, 45, 82, 83, 139, 175, 330, 353, 354, 355, 357, 374, 378, and 393. In some embodiments, the ACE2 extracellular domain or fragment thereof comprises two or more point mutations at any of positions 24, 27, 28, 30, 31, 34, 35, 37, 38, 41, 42, 45, 82, 83, 139, 175, 330, 353, 354, 355, 357, 374, 378, and 393 of SEQ ID NO: 5 or SEQ ID NO: 7. In some embodiments, the ACE2 extracellular domain or fragment thereof comprises two or more point mutations selected from the group consisting of D30E, K31T, H34Q, and D38E. In some embodiments, the ACE2 extracellular domain or fragment thereof comprises a point mutation selected from the group consisting of M82N, M82A, M82D, and M82S, and one or more point mutations selected from the group consisting of D30E, K31T, H34Q, and D38E. In some embodiments, the ACE2 extracellular domain or fragment thereof comprises a point mutation selected from the group consisting of M82N, M82A, M82D, and M82S and a point mutation selected from the group consisting of Q139S, Q139A, and Q139V. In some embodiments, the ACE2 extracellular domain or fragment thereof comprises a point mutation selected from the group consisting of M82N, M82A, M82D, and M82S and a point mutation selected from the group consisting of Q139S, Q139A, and Q139V. In some embodiments, the ACE2 extracellular domain or fragment thereof comprises a point mutation selected from the group consisting of M82N, M82A, M82D, and M82S and a point mutation selected from the group consisting of Q175S, Q175A, and Q175V. In some embodiments, the ACE2 extracellular domain or fragment thereof comprises a point mutation selected from the group consisting of M82N, M82A, M82D, and M82S and a point mutation selected from the group consisting of H374S, H374A, and H374V. In some embodiments, the ACE2 extracellular domain or fragment thereof comprises a point mutation selected from the group consisting of M82N, M82A, M82D, and M82S and a point mutation selected from the group consisting of H378S, H378A, and H378V. In some embodiments, the ACE2 extracellular domain or fragment thereof comprises a point mutation selected from the group consisting of Q139S, Q139A, and Q139V and a point mutation selected from the group consisting of H374S, H374A, and H374V. In some embodiments, the ACE2 extracellular domain or fragment thereof comprises a point mutation selected from the group consisting of Q175S, Q175A, and Q175V and a point mutation selected from the group consisting of H374S, H374A, and H374V. In some embodiments, the ACE2 extracellular domain or fragment thereof comprises a point mutation selected from the group consisting of Q139S, Q139A, and Q139V and a point mutation selected from the group consisting of H378S, H378A, and H378V. In some embodiments, the ACE2 extracellular domain or fragment thereof comprises a point mutation selected from the group consisting of Q175S, Q175A, and Q175V and a point mutation selected from the group consisting of H378S, H378A, and H378V.

In some embodiments, the ACE2 extracellular domain or fragment thereof comprises three or more point mutations at any of positions 24, 27, 28, 30, 31, 34, 35, 37, 38, 41, 42, 45, 82, 83, 139, 175, 330, 353, 354, 355, 357, 374, 378, and 393. In some embodiments, the ACE2 extracellular domain or fragment thereof comprises three or more point mutations at any of positions 24, 27, 28, 30, 31, 34, 35, 37, 38, 41, 42, 45, 82, 83, 139, 175, 330, 353, 354, 355, 357, 374, 378, and 393 of SEQ ID NO: 5 or SEQ ID NO: 7. In some embodiments, the ACE2 extracellular domain or fragment thereof comprises: (i) a point mutation selected from the group consisting of M82N, M82A, M82D, and M82S; (ii) a point mutation selected from the group consisting of Q139S, Q139A, and Q139V; and (iii) a point mutation selected from the group consisting of H374S, H374A, and H374V. In some embodiments, the ACE2 extracellular domain or fragment thereof comprises: (i) a point mutation selected from the group consisting of M82N, M82A, M82D, and M82S; (ii) a point mutation selected from the group consisting of Q175S, Q175A, and Q175V; and (iii) a point mutation selected from the group consisting of H378S, H378A, and H378V. In some embodiments, the ACE2 extracellular domain or fragment thereof comprises: (i) a point mutation selected from the group consisting of M82N, M82A, M82D, and M82S; (ii) a point mutation selected from the group consisting of Q175S, Q175A, and Q175V; and (iii) a point mutation selected from the group consisting of H378S, H378A, and H378V.

In some embodiments, the ACE2 extracellular domain or fragment thereof comprises four or more point mutations at any of positions 24, 27, 28, 30, 31, 34, 35, 37, 38, 41, 42, 45, 82, 83, 139, 175, 330, 353, 354, 355, 357, 374, 378, and 393. In some embodiments, the ACE2 extracellular domain or fragment thereof comprises four or more point mutations at any of positions 24, 27, 28, 30, 31, 34, 35, 37, 38, 41, 42, 45, 82, 83, 139, 175, 330, 353, 354, 355, 357, 374, 378, and 393 of SEQ ID NO: 5 or SEQ ID NO: 7. In some embodiments, the ACE2 extracellular domain or fragment thereof comprises: (i) a point mutation selected from the group consisting of M82N, M82A, M82D, and M82S; (ii) a point mutation selected from the group consisting of Q139S, Q139A, and Q139V; (iii) a point mutation selected from the group consisting of H374S, H374A, and H374V; and (iv) a point mutation selected from the group consisting of H378S, H378A, and H378V. In some embodiments, the ACE2 extracellular domain or fragment thereof comprises: (i) a point mutation selected from the group consisting of M82N, M82A, M82D, and M82S; (ii) a point mutation selected from the group consisting of Q175S, Q175A, and Q175V; (iii) a point mutation selected from the group consisting of H374S, H374A, and H374V; and (iv) a point mutation selected from the group consisting of H378S, H378A, and H378V. In some embodiments, ACE2 extracellular domain comprises the point mutations M82N, Q139A, H374S, and H378S. In some embodiments, the ACE2 extracellular domain or fragment thereof comprises the amino acid sequence of SEQ ID NO: 5 or SEQ ID NO: 7 with the point mutations M82N, Q139A, H374S, and H378S.

In some embodiments, the ACE2 extracellular domain or fragment thereof comprises five or more point mutations at any of positions 24, 27, 28, 30, 31, 34, 35, 37, 38, 41, 42, 45, 82, 83, 139, 175, 330, 353, 354, 355, 357, 374, 378, and 393. In some embodiments, the ACE2 extracellular domain or fragment thereof comprises five or more point mutations at any of positions 24, 27, 28, 30, 31, 34, 35, 37, 38, 41, 42, 45, 82, 83, 139, 175, 330, 353, 354, 355, 357, 374, 378, and 393 of SEQ ID NO: 5 or SEQ ID NO: 7. In some embodiments, the ACE2 extracellular domain or fragment thereof comprises: (i) a point mutation selected from the group consisting of M82N, M82A, M82D, and M82S; (ii) a point mutation selected from the group consisting of Q139S, Q139A, and Q139V; (iii) a point mutation selected from the group consisting of Q175S, Q175A, and Q175V; (iv) a point mutation selected from the group consisting of H374S, H374A, and H374V; and (v) a point mutation selected from the group consisting of H378S, H378A, and H378V.

Others have demonstrated that fragments of the ACE2 Extracellular Domain, hereinafter referred to as ACE2 ECD fragments, as isolated non-Fc molecules can retain as great or greater ACE2 enzymatic activity compared with native ACE2 and because of the smaller size can improve transit to and through peripheral tissues such as the kidney. For example, Wysocki et al. generated two short recombinant ACE2 variants, 1-605AA and 1-619AA, that had a molecular size of ˜70 kDa compared with the molecular size of ˜100 kDa for native ACE2 (Wysocki, Biomolecules 2019, 9, 886). Wysocki et al. demonstrated that ACE2 activity was recovered in harvested kidneys from ACE2-deficient mice infused with the truncated ACE2 ECD fragment 1-619, but not in controls, and that the kidneys of ACE2-null mice infused with ACE2 ECD fragment 1-619 studied ex vivo formed more Ang (1-7) from exogenous Ang II than those infused with vehicle. In some embodiments, the ACE2-Fc fusion proteins of the present invention comprise truncated ACE2 ECD fragments. In some embodiments, the truncated ACE2 ECD fragments are associated with the same, nearly the same, or even greater ACE2 enzymatic activity compared with native ACE2. In some embodiments, the truncated ACE2 ECD fragments are associated with increased delivery to peripheral tissues compared with larger, intact native ACE2 or ACE2 ECD. In some embodiments, the truncated ACE2 ECD fragments comprised in the current invention are between 601 and 619 amino acids.

Studies have shown that the ACE2 amino acids 18-615 appear to be sufficient for SARS S protein binding, which also covers the peptidase domain necessary for ACE2 enzymatic function (Kruse, F1000Research, 2020). In some embodiments, the truncated ACE2 ECD fragments comprised in the current invention are amino acids 18-615 (SEQ ID NO: 8).

### Fc Domains and Fragments Thereof

In some embodiments, the ACE2-Fc fusion proteins of the present disclosure comprise one or more Fc domains. In some embodiments, the Fc domains may comprise Fc fragments or Fc partial fragments

### Fc Fragments

The term “Fc fragment” refers to the protein region or protein folded structure that is routinely found at the carboxy terminus of immunoglobulins. The Fc fragment can be isolated from the Fab fragment of a monoclonal antibody through the use of enzymatic digestion, for example papain digestion, which is an incomplete and imperfect process (See Mihaesco C et al., Journal of Experimental Medicine, Vol 127, 431-453 (1968)). In conjunction with the Fab fragment (containing the antigen binding domain), the Fc fragment constitutes the holo-antibody, meaning here the complete antibody. The Fc fragment consists of the carboxy terminal portions of the antibody heavy chains. Each of the chains in an Fc fragment is between about 220-265 amino acids in length and the chains are often linked via a disulfide bond. The Fc fragment often contains one or more independent structural folds or functional subdomains. In particular, the Fc fragment encompasses an Fc domain, defined herein as the minimum structure that binds an Fc receptor. An isolated Fc fragment is comprised of two Fc fragment monomers (e.g., the two carboxy terminal portions of the antibody heavy chains; further defined herein) that are dimerized. When two Fc fragment monomers associate, the resulting Fc fragment has complement and/or Fc receptor binding activity.

### Fc Partial Fragment

An “Fc partial fragment” is a domain comprising less than the entire Fc fragment of an antibody, yet which retains sufficient structure to have the same activity as the Fc fragment, including Fc receptor binding activity and/or complement binding activity. An Fc partial fragment may therefore lack part or all of a hinge region, part or all of a CH2 domain, part or all of a CH3 domain, and/or part or all of a CH4 domain, depending on the isotype of the antibody from which the Fc partial domain is derived. For example, an Fc partial fragment includes a molecule comprising the CH2 and CH3 domains of IgG1. In this example, the Fc partial fragment lacks the hinge domain present in IgG1.

Fc partial fragments are comprised of two Fc partial fragment monomers. When two such Fc partial fragment monomers associate, the resulting Fc partial fragment has Fc receptor binding activity and/or complement binding activity.

### Fc Domain

As used herein, “Fe domain” describes the minimum region (in the context of a larger polypeptide) or smallest protein folded structure (in the context of an isolated protein) that can bind to or be bound by an Fc receptor (FcR). In both an Fc fragment and an Fc partial fragment, the Fc domain is the minimum binding region that allows binding of the molecule to an Fc receptor. While an Fc domain can be limited to a discrete homodimeric polypeptide that is bound by an Fc receptor, it will also be clear that an Fc domain can be a part or all of an Fc fragment, as well as part or all of an Fc partial fragment. When the term “Fe domains” is used in this invention it will be recognized by a skilled artisan as meaning more than one Fc domain. An Fc domain is comprised of two Fc domain monomers. As further defined herein, when two such Fc domain monomers associate, the resulting Fc domain has Fc receptor binding activity and/or complement binding activity. Thus, an Fc domain is a dimeric structure that can bind complement and/or an Fc receptor.

### Fc Partial Domain

As used herein, “Fc partial domain” describes a portion of an Fc domain. Fc partial domains include the individual heavy chain constant region domains (e.g., CH1, CH2, CH3 and CH4 domains) and hinge regions of the different immunoglobulin classes and subclasses. Thus, human Fc partial domains of the present invention include the CH1 domain of IgG1, the CH2 domain of IgG1, the CH3 domain of IgG1, and the hinge regions of IgG1 and IgG2. The corresponding Fc partial domains in other species will depend on the immunoglobulins present in that species and the naming thereof. Preferably, the Fc partial domains of the current invention include CH1, CH2 and hinge domains of IgG1 and the hinge domain of IgG2. The Fc partial domain of the present invention may further comprise a combination of more than one of these domains and hinges. However, the individual Fc partial domains of the present invention and combinations thereof lack the ability to bind an FcR. Therefore, the Fc partial domains and combinations thereof comprise less than an Fc domain. Fc partial domains may be linked together to form a peptide that has complement and/or Fc receptor binding activity, thus forming an Fc domain. In the present invention, Fc partial domains are used with Fc domains as the building blocks to create the multi-Fc therapeutics used in accordance with the methods of the present invention, as described herein. Each Fc partial domain is comprised of two Fc partial domain monomers. When two such Fc partial domain monomers associate, an Fc partial domain is formed.

As indicated above, each of Fc fragments, Fc partial fragments, Fc domains and Fc partial domains are dimeric proteins or domains. Thus, each of these molecules is comprised of two monomers that associate to form the dimeric protein or domain. While the characteristics and activity of the homodimeric forms was discussed above the monomeric peptides are discussed as follows.

### Fc Fragment Monomer

As used herein, an “Fc fragment monomer” is a single chain protein that, when associated with another Fc fragment monomer, comprises an Fc fragment. The Fc fragment monomer is thus the carboxy-terminal portion of one of the antibody heavy chains that make up the Fc fragment of a holo-antibody (e.g., the contiguous portion of the heavy chain that includes the hinge region, CH2 domain and CH3 domain of IgG). In one embodiment, the Fc fragment monomer comprises, at a minimum, one chain of a hinge region (a hinge monomer), one chain of a CH2 domain (a CH2 domain monomer) and one chain of a CH3 domain (a CH3 domain monomer), contiguously linked to form a peptide. In some embodiments, the CH2, CH3 and hinge domains are from different isotypes.

### Fc Domain Monomers

In some embodiments, the ACE2-Fc fusion protein comprises an Fc domain monomer. As used herein, “Fc domain monomer” describes the single chain protein that, when associated with another Fc domain monomer, comprises an Fc domain that can bind to complement and/or canonical Fc receptors. The association of two Fc domain monomers creates one Fc domain.

In some embodiments, the ACE2-Fc fusion proteins comprise an IgG1 Fc domain monomer. In some embodiments, the IgG1 Fc domain monomer comprises, from amino to carboxy-terminus, an IgG1 hinge, IgG1 CH2 domain, and IgG1 CH3 domain. In some embodiments, the IgG1 Fc domain monomer comprises an amino acid sequence that is at least 80%, at least 85%, at least 90%, at least 95%, at least 96%, at least 97%, at least 98%, or at least 99% identical to SEQ ID NO: 39. In some embodiments, the IgG1 Fc domain monomer comprises or consists of SEQ ID NO: 39.

In some embodiments, the ACE2-Fc fusion protein comprises an IgG2 Fc domain monomer. In some embodiments, the IgG2 Fc domain monomer comprises, from amino to carboxy-terminus, an IgG2 hinge, IgG2 CH2 domain, and IgG2 CH3 domain. In some embodiments, the IgG2 Fc domain monomer comprises an amino acid sequence that is at least 80%, at least 85%, at least 90%, at least 95%, at least 96%, at least 97%, at least 98%, or at least 99% identical to SEQ ID NO: 40. In some embodiments, the IgG2 Fc domain monomer comprises or consists of SEQ ID NO: 40.

In some embodiments, the ACE2-Fc fusion protein comprises an IgG3 Fc domain monomer. In some embodiments, the IgG3 Fc domain monomer comprises, from amino to carboxy-terminus, an IgG3 hinge, IgG3 CH2 domain, and IgG3 CH3 domain. In some embodiments, the IgG3 Fc domain monomer comprises an amino acid sequence that is at least 80%, at least 85%, at least 90%, at least 95%, at least 96%, at least 97%, at least 98%, or at least 99% identical to SEQ ID NO: 41. In some embodiments, the IgG3 Fc domain monomer comprises or consists of SEQ ID NO: 41.

In some embodiments, the ACE2-Fc fusion protein comprises an IgG4 Fc domain monomer. In some embodiments, the IgG4 Fc domain monomer comprises, from amino to carboxy-terminus, an IgG4 hinge, IgG4 CH2 domain, and IgG4 CH3 domain. In some embodiments, the IgG4 Fc domain monomer comprises an amino acid sequence that is at least 80%, at least 85%, at least 90%, at least 95%, at least 96%, at least 97%, at least 98%, or at least 99% identical to SEQ ID NO: 42. In some embodiments, the IgG4 Fc domain monomer comprises or consists of SEQ ID NO: 42.

In some embodiments, the Fc domains in the ACE2-Fc fusion proteins of the present disclosure demonstrate reduced binding to one or more low affinity Fcγ receptors (e.g., FcγRIIA, FcγRIIB, FcγRIIC, FcγRIIIA, or FcγRIIIB) compared to a wild type IgG1 Fc domain. In some embodiments, the Fc domain is an IgG4 Fc domain. In some embodiments, the Fc domain is an IgG1 or IgG3 Fc domain that has been mutated to reduce binding to one or more low affinity Fcγ receptors. One skilled in the art will know that there are many described techniques for decreasing IgG Fc binding to FcγRs, including the commonly employed combination of Leu234Ala and Leu235Ala (commonly called LALA mutations). Some of these known techniques are summarized by Saunders, Front. Immunol., 2019.

Exemplary Fc domains of the present disclosure are shown in Table 3 below.

TABLE3
Exemplary Fc Domains

| | | SEQ |
|---|---|---|
| Fc domain | Amino Acid Sequence | ID NO: |
| Human IgG1 | EPKSCDKTHTCPPCPAPELLGGPSVFLFPPK | 39 |
| | PKDTLMISRTPEVTCVVVDVSHEDPEVKFNW | |
| | YVDGVEVHNAKTKPREEQYNSTYRVVSVLTV | |
| | LHQDWLNGKEYKCKVSNKALPAPIEKTISKA | |
| | KGQPREPQVYTLPPSREEMTKNQVSLTCLVK | |
| | GFYPSDIAVEWESNGQPENNYKTTPPVLDSD | |
| | GSFFLYSKLTVDKSRWQQGNVFSCSVMHEAL | |
| | HNHYTQKSLSLSPGK | |
| Human IgG2 | ERKCCVECPPCPAPPVAGPSVFLFPPKPKDT | 40 |
| | LMISRTPEVTCVVVDVSHEDPEVQFNWYVDG | |
| | VEVHNAKTKPREEQFNSTFRVVSVLTVVHQD | |
| | WLNGKEYKCKVSNKGLPAPIEKTISKTKGQP | |
| | REPQVYTLPPSREEMTKNQVSLTCLVKGFYP | |
| | SDISVEWESNGQPENNYKTTPPMLDSDGSFF | |
| | LYSKLTVDKSRWQQGNVFSCSVMHEALHNHY | |
| | TQKSLSLSPGK | |
| Human IgG3 | ELKTPLGDTTHTCPRCPEPKSCDTPPPCPRC | 41 |
| | PEPKSCDTPPPCPRCPEPKSCDTPPPCPRCP | |
| | APELLGGPSVFLFPPKPKDTLMISRTPEVTC | |
| | VVVDVSHEDPEVQFKWYVDGVEVHNAKTKPR | |
| | EEQYNSTFRVVSVLTVLHQDWLNGKEYKCKV | |
| | SNKALPAPIEKTISKTKGQPREPQVYTLPPS | |
| | REEMTKNQVSLTCLVKGFYPSDIAVEWESSG | |
| | QPENNYNTTPPMLDSDGSFFLYSKLTVDKSR | |
| | WQQGNIFSCSVMHEALHNRFTQKSLSLSPGK | |
| Human IgG4 | ESKYGPPCPSCPAPEFLGGPSVFLFPPKPKD | 42 |
| | TLMISRTPEVTCVVVDVSQEDPEVQFNWYVD | |
| | GVEVHNAKTKPREEQFNSTYRVVSVLTVLHQ | |
| | DWLNGKEYKCKVSNKGLPSSIEKTISKAKGQ | |
| | PREPQVYTLPPSQEEMTKNQVSLTCLVKGFY | |
| | PSDIAVEWESNGQPENNYKTTPPVLDSDGSF | |
| | FLYSRLTVDKSRWQEGNVFSCSVMHELAHNH | |
| | YTQKSLSLSLGK | |

### ACE2-Fc Fusion Proteins

In some embodiments, the ACE2-Fc fusion proteins disclosed herein include the amino acid sequence of ACE2 or a fragment or variant thereof fused to the amino acid sequence of an antibody fragment, e.g., the Fc portion of an antibody.

In some embodiments, the ACE2-Fc fusion proteins or polypeptides disclosed herein may include an amino acid tag sequence, which may be utilized for purifying and or identifying the fusion protein. Suitable amino acid tag sequences may include, but are not limited to, histidine tag sequences, FLAG tag sequences, GST tag sequences, and the like.

The ACE2-Fc fusion proteins disclosed herein may comprise a linker sequence. As used herein, the term “linker” refers to a polypeptide sequence that joins two protein domains together. Suitable linker sequences may include amino acid sequences of 5, 6, 7, 8, 9, 10, 11, 12, 13, 14, or 15 amino acids or more, or a range bounded by any of these values (e.g., a linker of 5-15 amino acids). In some embodiments, the linker sequence comprises only glycine and serine residues.

In some embodiments, the present disclosure provides an ACE2-Fc fusion protein comprising an ACE2 domain or fragment thereof; and one or more Fc domains. In some embodiments, ACE2-Fc fusion protein further comprises a signal peptide, wherein the signal peptide comprises an amino acid sequence that is at least 80%, 85%, 90%, 95%, 96%, 97%, 98%, or 99% identical to SEQ ID NO: 1. In some embodiments, the signal peptide comprises or consists of SEQ ID NO: 1. In some embodiments, the signal peptide is cleaved from the ACE2-Fc fusion protein. In some embodiments, the ACE2 domain is an ACE2 extracellular domain or fragment thereof. In some embodiments, the ACE2 extracellular domain or fragment thereof comprises an amino acid sequence that is at least 80%, 85%, 90%, 95%, 96%, 97%, 98%, or 99% identical to SEQ ID NO: 6. In some embodiments the ACE2 extracellular domain or fragment thereof comprises or consists of SEQ ID NO: 6. In some embodiments, the one or more Fc domains is an IgG1 Fc domain. In some embodiments, the IgG1 Fc domain comprises an amino acid sequence that is at least 80%, 85%, 90%, 95%, 96%, 97%, 98%, or 99% identical to SEQ ID NO: 39. In some embodiments, the IgG1 Fc domain comprises or consists of SEQ ID NO: 39.

In some embodiments, the present disclosure provides an ACE2-Fc fusion protein comprising an ACE2 domain or fragment thereof; and one or more Fc domains. In some embodiments, ACE2-Fc fusion protein further comprises a signal peptide, wherein the signal peptide comprises an amino acid sequence that is at least 80%, 85%, 90%, 95%, 96%, 97%, 98%, or 99% identical to SEQ ID NO: 1. In some embodiments, the signal peptide comprises or consists of SEQ ID NO: 1. In some embodiments, the signal peptide is cleaved from the ACE2-Fc fusion protein. In some embodiments, the ACE2 domain is an ACE2 extracellular domain or fragment thereof. In some embodiments, the ACE2 extracellular domain or fragment thereof comprises an amino acid sequence that is at least 80%, 85%, 90%, 95%, 96%, 97%, 98%, or 99% identical to SEQ ID NO: 6. In some embodiments the ACE2 extracellular domain or fragment thereof comprises or consists of SEQ ID NO: 6. In some embodiments, the one or more Fc domains is an IgG2 Fc domain. In some embodiments, the IgG2 Fc domain comprises an amino acid sequence that is at least 80%, 85%, 90%, 95%, 96%, 97%, 98%, or 99% identical to SEQ ID NO: 40. In some embodiments, the IgG2 Fc domain comprises or consists of SEQ ID NO: 40.

In some embodiments, the present disclosure provides an ACE2-Fc fusion protein comprising an ACE2 domain or fragment thereof; and one or more Fc domains. In some embodiments, ACE2-Fc fusion protein further comprises a signal peptide, wherein the signal peptide comprises an amino acid sequence that is at least 80%, 85%, 90%, 95%, 96%, 97%, 98%, or 99% identical to SEQ ID NO: 1. In some embodiments, the signal peptide comprises or consists of SEQ ID NO: 1. In some embodiments, the signal peptide is cleaved from the ACE2-Fc fusion protein. In some embodiments, the ACE2 domain is an ACE2 extracellular domain or fragment thereof. In some embodiments, the ACE2 extracellular domain or fragment thereof comprises an amino acid sequence that is at least 80%, 85%, 90%, 95%, 96%, 97%, 98%, or 99% identical to SEQ ID NO: 6. In some embodiments the ACE2 extracellular domain or fragment thereof comprises or consists of SEQ ID NO: 6. In some embodiments, the one or more Fc domains is an IgG3 Fc domain. In some embodiments, the IgG3 Fc domain comprises an amino acid sequence that is at least 80%, 85%, 90%, 95%, 96%, 97%, 98%, or 99% identical to SEQ ID NO: 41. In some embodiments, the IgG3 Fc domain comprises or consists of SEQ ID NO: 41.

In some embodiments, the present disclosure provides an ACE2-Fc fusion protein comprising an ACE2 domain or fragment thereof; and one or more Fc domains. In some embodiments, ACE2-Fc fusion protein further comprises a signal peptide, wherein the signal peptide comprises an amino acid sequence that is at least 80%, 85%, 90%, 95%, 96%, 97%, 98%, or 99% identical to SEQ ID NO: 1. In some embodiments, the signal peptide comprises or consists of SEQ ID NO: 1. In some embodiments, the signal peptide is cleaved from the ACE2-Fc fusion protein. In some embodiments, the ACE2 domain is an ACE2 extracellular domain or fragment thereof. In some embodiments, the ACE2 extracellular domain or fragment thereof comprises an amino acid sequence that is at least 80%, 85%, 90%, 95%, 96%, 97%, 98%, or 99% identical to SEQ ID NO: 6. In some embodiments the ACE2 extracellular domain or fragment thereof comprises or consists of SEQ ID NO: 6. In some embodiments, the one or more Fc domains is an IgG4 Fc domain. In some embodiments, the IgG4 Fc domain comprises an amino acid sequence that is at least 80%, 85%, 90%, 95%, 96%, 97%, 98%, or 99% identical to SEQ ID NO: 42. In some embodiments, the IgG4 Fc domain comprises or consists of SEQ ID NO: 42.

In some embodiments, the present disclosure provides an ACE2-Fc fusion protein comprising an ACE2 domain or fragment thereof; and one or more Fc domains. In some embodiments, ACE2-Fc fusion protein further comprises a signal peptide, wherein the signal peptide comprises an amino acid sequence that is at least 80%, 85%, 90%, 95%, 96%, 97%, 98%, or 99% identical to SEQ ID NO: 1. In some embodiments, the signal peptide comprises or consists of SEQ ID NO: 1. In some embodiments, the signal peptide is cleaved from the ACE2-Fc fusion protein. In some embodiments, the ACE2 domain is an ACE2 extracellular domain or fragment thereof. In some embodiments, the ACE2 extracellular domain or fragment thereof comprises an amino acid sequence that is at least 80%, 85%, 90%, 95%, 96%, 97%, 98%, or 99% identical to SEQ ID NO: 8. In some embodiments the ACE2 extracellular domain or fragment thereof comprises or consists of SEQ ID NO: 8. In some embodiments, the one or more Fc domains is an IgG1 Fc domain. In some embodiments, the IgG1 Fc domain comprises an amino acid sequence that is at least 80%, 85%, 90%, 95%, 96%, 97%, 98%, or 99% identical to SEQ ID NO: 39. In some embodiments, the IgG1 Fc domain comprises or consists of SEQ ID NO: 39.

In some embodiments, the present disclosure provides an ACE2-Fc fusion protein comprising an ACE2 domain or fragment thereof; and one or more Fc domains. In some embodiments, ACE2-Fc fusion protein further comprises a signal peptide, wherein the signal peptide comprises an amino acid sequence that is at least 80%, 85%, 90%, 95%, 96%, 97%, 98%, or 99% identical to SEQ ID NO: 1. In some embodiments, the signal peptide comprises or consists of SEQ ID NO: 1. In some embodiments, the signal peptide is cleaved from the ACE2-Fc fusion protein. In some embodiments, the ACE2 domain is an ACE2 extracellular domain or fragment thereof. In some embodiments, the ACE2 extracellular domain or fragment thereof comprises an amino acid sequence that is at least 80%, 85%, 90%, 95%, 96%, 97%, 98%, or 99% identical to SEQ ID NO: 8. In some embodiments the ACE2 extracellular domain or fragment thereof comprises or consists of SEQ ID NO: 8. In some embodiments, the one or more Fc domains is an IgG2 Fc domain. In some embodiments, the IgG2 Fc domain comprises an amino acid sequence that is at least 80%, 85%, 90%, 95%, 96%, 97%, 98%, or 99% identical to SEQ ID NO: 40. In some embodiments, the IgG1 Fc domain comprises or consists of SEQ ID NO: 40.

In some embodiments, the present disclosure provides an ACE2-Fc fusion protein comprising an ACE2 domain or fragment thereof; and one or more Fc domains. In some embodiments, ACE2-Fc fusion protein further comprises a signal peptide, wherein the signal peptide comprises an amino acid sequence that is at least 80%, 85%, 90%, 95%, 96%, 97%, 98%, or 99% identical to SEQ ID NO: 1. In some embodiments, the signal peptide comprises or consists of SEQ ID NO: 1. In some embodiments, the signal peptide is cleaved from the ACE2-Fc fusion protein. In some embodiments, the ACE2 domain is an ACE2 extracellular domain or fragment thereof. In some embodiments, the ACE2 extracellular domain or fragment thereof comprises an amino acid sequence that is at least 80%, 85%, 90%, 95%, 96%, 97%, 98%, or 99% identical to SEQ ID NO: 8. In some embodiments the ACE2 extracellular domain or fragment thereof comprises or consists of SEQ ID NO: 8. In some embodiments, the one or more Fc domains is an IgG3 Fc domain. In some embodiments, the IgG3 Fc domain comprises an amino acid sequence that is at least 80%, 85%, 90%, 95%, 96%, 97%, 98%, or 99% identical to SEQ ID NO: 41. In some embodiments, the IgG3 Fc domain comprises or consists of SEQ ID NO: 41.

In some embodiments, the present disclosure provides an ACE2-Fc fusion protein comprising an ACE2 domain or fragment thereof; and one or more Fc domains. In some embodiments, ACE2-Fc fusion protein further comprises a signal peptide, wherein the signal peptide comprises an amino acid sequence that is at least 80%, 85%, 90%, 95%, 96%, 97%, 98%, or 99% identical to SEQ ID NO: 1. In some embodiments, the signal peptide comprises or consists of SEQ ID NO: 1. In some embodiments, the signal peptide is cleaved from the ACE2-Fc fusion protein. In some embodiments, the ACE2 domain is an ACE2 extracellular domain or fragment thereof. In some embodiments, the ACE2 extracellular domain or fragment thereof comprises an amino acid sequence that is at least 80%, 85%, 90%, 95%, 96%, 97%, 98%, or 99% identical to SEQ ID NO: 8. In some embodiments the ACE2 extracellular domain or fragment thereof comprises or consists of SEQ ID NO: 8. In some embodiments, the one or more Fc domains is an IgG4 Fc domain. In some embodiments, the IgG4 Fc domain comprises an amino acid sequence that is at least 80%, 85%, 90%, 95%, 96%, 97%, 98%, or 99% identical to SEQ ID NO: 42. In some embodiments, the IgG4 Fc domain comprises or consists of SEQ ID NO: 42.

Exemplary ACE2-Fc fusion proteins are shown in Table 4 below. The signal peptide sequence is underlined.

TABLE4
ACE2-Fc Fusion Proteins

| Fc domain | Amino Acid Sequence | SEQ ID: |
|---|---|---|
| ACE2(ECD)-IgG1 | METDTLLLWVLLLWVPGSTGQSTIEEQAKTFLDKFNHEAEDLFYQS | 43 |
| | SLASWNYNTNITEENVQNMNNAGDKWSAFLKEQSTLAQMYPLQEIQ | |
| | NLTVKLQLQALQQNGSSVLSEDKSKRLNTILNTMSTIYSTGKVCNP | |
| | DNPQECLLLEPGLNEIMANSLDYNERLWAWESWRSEVGKQLRPLYE | |
| | EYVVLKNEMARANHYEDYGDYWRGDYEVNGVDGYDYSRGQLIEDVE | |
| | HTFEEIKPLYEHLHAYVRAKLMNAYPSYISPIGCLPAHLLGDMWGR | |
| | FWTNLYSLTVPFGQKPNIDVTDAMVDQAWDAQRIFKEAEKFFVSVG | |
| | LPNMTQGFWENSMLTDPGNVQKAVCHPTAWDLGKGDFRILMCTKVT | |
| | MDDFLTAHHEMGHIQYDMAYAAQPFLLRNGANEGFHEAVGEIMSLS | |
| | AATPKHLKSIGLLSPDFQEDNETEINFLLKQALTIVGTLPFTYMLE | |
| | KWRWMVFKGEIPKDQWMKKWWEMKREIVGVVEPVPHDETYCDPASL | |
| | FHVSNDYSFIRYYTRTLYQFQFQEALCQAAKHEGPLHKCDISNSTE | |
| | AGQKLFNMLRLGKSEPWTLALENVVGAKNMNVRPLLNYFEPLFTWL | |
| | KDQNKNSFVGWSTDWSPYADQSIKVRISLKSALGDKAYEWNDNEMY | |
| | LFRSSVAYAMRQYFLKVKNQMILFGEEDVRVANLKPRISFNFFVTA | |
| | PKNVSDIIPRTEVEKAIRMSRSRINDAFRLNDNSLEFLGIQPTLGP | |
| | PNQPPVSEPKSCDKTHTCPPCPAPELLGGPSVFLFPPKPKDTLMIS | |
| | RTPEVTCVVVDVSHEDPEVKFNWYVDGVEVHNAKTKPREEQYNSTY | |
| | RVVSVLTVLHQDWLNGKEYKCKVSNKALPAPIEKTISKAKGQPREP | |
| | QVYTLPPSREEMTKNQVSLTCLVKGFYPSDIAVEWESNGQPENNYK | |
| | TTPPVLDSDGSFFLYSKLTVDKSRWQQGNVFSCSVMHEALHNHYTQ | |
| | KSLSLSPGK | |
| ACE2(ECD)-IgG2 | METDTLLLWVLLLWVPGSTGQSTIEEQAKTFLDKFNHEAEDLFYQS | 44 |
| | SLASWNYNTNITEENVQNMNNAGDKWSAFLKEQSTLAQMYPLQEIQ | |
| | NLTVKLQLQALQQNGSSVLSEDKSKRLNTILNTMSTIYSTGKVCNP | |
| | DNPQECLLLEPGLNEIMANSLDYNERLWAWESWRSEVGKQLRPLYE | |
| | EYVVLKNEMARANHYEDYGDYWRGDYEVNGVDGYDYSRGQLIEDVE | |
| | HTFEEIKPLYEHLHAYVRAKLMNAYPSYISPIGCLPAHLLGDMWGR | |
| | FWTNLYSLTVPFGQKPNIDVTDAMVDQAWDAQRIFKEAEKFFVSVG | |
| | LPNMTQGFWENSMLTDPGNVQKAVCHPTAWDLGKGDFRILMCTKVT | |
| | MDDFLTAHHEMGHIQYDMAYAAQPFLLRNGANEGFHEAVGEIMSLS | |
| | AATPKHLKSIGLLSPDFQEDNETEINFLLKQALTIVGTLPFTYMLE | |
| | KWRWMVFKGEIPKDQWMKKWWEMKREIVGVVEPVPHDETYCDPASL | |
| | FHVSNDYSFIRYYTRTLYQFQFQEALCQAAKHEGPLHKCDISNSTE | |
| | AGQKLFNMLRLGKSEPWTLALENVVGAKNMNVRPLLNYFEPLFTWL | |
| | KDQNKNSFVGWSTDWSPYADQSIKVRISLKSALGDKAYEWNDNEMY | |
| | LFRSSVAYAMRQYFLKVKNQMILFGEEDVRVANLKPRISFNFFVTA | |
| | PKNVSDIIPRTEVEKAIRMSRSRINDAFRLNDNSLEFLGIQPTLGP | |
| | PNQPPVSERKCCVECPPCPAPPVAGPSVFLFPPKPKDTLMISRTPE | |
| | VTCVVVDVSHEDPEVQFNWYVDGVEVHNAKTKPREEQFNSTFRVVS | |
| | VLTVVHQDWLNGKEYKCKVSNKGLPAPIEKTISKTKGQPREPQVYT | |
| | LPPSREEMTKNQVSLTCLVKGFYPSDISVEWESNGQPENNYKTTPP | |
| | MLDSDGSFFLYSKLTVDKSRWQQGNVFSCSVMHEALHNHYTQKSLS | |
| | LSPGK | |
| ACE2(ECD)-IgG3 | METDTLLLWVLLLWVPGSTGQSTIEEQAKTFLDKFNHEAEDLFYQS | 45 |
| | SLASWNYNTNITEENVQNMNNAGDKWSAFLKEQSTLAQMYPLQEIQ | |
| | NLTVKLQLQALQQNGSSVLSEDKSKRLNTILNTMSTIYSTGKVCNP | |
| | DNPQECLLLEPGLNEIMANSLDYNERLWAWESWRSEVGKQLRPLYE | |
| | EYVVLKNEMARANHYEDYGDYWRGDYEVNGVDGYDYSRGQLIEDVE | |
| | HTFEEIKPLYEHLHAYVRAKLMNAYPSYISPIGCLPAHLLGDMWGR | |
| | FWTNLYSLTVPFGQKPNIDVTDAMVDQAWDAQRIFKEAEKFFVSVG | |
| | LPNMTQGFWENSMLTDPGNVQKAVCHPTAWDLGKGDFRILMCTKVT | |
| | MDDFLTAHHEMGHIQYDMAYAAQPFLLRNGANEGFHEAVGEIMSLS | |
| | AATPKHLKSIGLLSPDFQEDNETEINFLLKQALTIVGTLPFTYMLE | |
| | KWRWMVFKGEIPKDQWMKKWWEMKREIVGVVEPVPHDETYCDPASL | |
| | FHVSNDYSFIRYYTRTLYQFQFQEALCQAAKHEGPLHKCDISNSTE | |
| | AGQKLFNMLRLGKSEPWTLALENVVGAKNMNVRPLLNYFEPLFTWL | |
| | KDQNKNSFVGWSTDWSPYADQSIKVRISLKSALGDKAYEWNDNEMY | |
| | LFRSSVAYAMRQYFLKVKNQMILFGEEDVRVANLKPRISFNFFVTA | |
| | PKNVSDIIPRTEVEKAIRMSRSRINDAFRLNDNSLEFLGIQPTLGP | |
| | PNQPPVSELKTPLGDTTHTCPRCPEPKSCDTPPPCPRCPEPKSCDT | |
| | PPPCPRCPEPKSCDTPPPCPRCPAPELLGGPSVFLFPPKPKDTLMI | |
| | SRTPEVTCVVVDVSHEDPEVQFKWYVDGVEVHNAKTKPREEQYNST | |
| | FRVVSVLTVLHQDWLNGKEYKCKVSNKALPAPIEKTISKTKGQPRE | |
| | PQVYTLPPSREEMTKNQVSLTCLVKGFYPSDIAVEWESSGQPENNY | |
| | NTTPPMLDSDGSFFLYSKLTVDKSRWQQGNIFSCSVMHEALHNRFT | |
| | QKSLSLSPGK | |
| ACE2(ECD)-IgG4 | METDTLLLWVLLLWVPGSTGQSTIEEQAKTFLDKFNHEAEDLFYQS | 46 |
| | SLASWNYNTNITEENVQNMNNAGDKWSAFLKEQSTLAQMYPLQEIQ | |
| | NLTVKLQLQALQQNGSSVLSEDKSKRLNTILNTMSTIYSTGKVCNP | |
| | DNPQECLLLEPGLNEIMANSLDYNERLWAWESWRSEVGKQLRPLYE | |
| | EYVVLKNEMARANHYEDYGDYWRGDYEVNGVDGYDYSRGQLIEDVE | |
| | HTFEEIKPLYEHLHAYVRAKLMNAYPSYISPIGCLPAHLLGDMWGR | |
| | FWTNLYSLTVPFGQKPNIDVTDAMVDQAWDAQRIFKEAEKFFVSVG | |
| | LPNMTQGFWENSMLTDPGNVQKAVCHPTAWDLGKGDFRILMCTKVT | |
| | MDDFLTAHHEMGHIQYDMAYAAQPFLLRNGANEGFHEAVGEIMSLS | |
| | AATPKHLKSIGLLSPDFQEDNETEINFLLKQALTIVGTLPFTYMLE | |
| | KWRWMVFKGEIPKDQWMKKWWEMKREIVGVVEPVPHDETYCDPASL | |
| | FHVSNDYSFIRYYTRTLYQFQFQEALCQAAKHEGPLHKCDISNSTE | |
| | AGQKLFNMLRLGKSEPWTLALENVVGAKNMNVRPLLNYFEPLFTWL | |
| | KDQNKNSFVGWSTDWSPYADQSIKVRISLKSALGDKAYEWNDNEMY | |
| | LFRSSVAYAMRQYFLKVKNQMILFGEEDVRVANLKPRISFNFFVTA | |
| | PKNVSDIIPRTEVEKAIRMSRSRINDAFRLNDNSLEFLGIQPTLGP | |
| | PNQPPVSESKYGPPCPSCPAPEFLGGPSVFLFPPKPKDTLMISRTP | |
| | EVTCVVVDVSQEDPEVQFNWYVDGVEVHNAKTKPREEQFNSTYRVV | |
| | SVLTVLHQDWLNGKEYKCKVSNKGLPSSIEKTISKAKGQPREPQVY | |
| | TLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSL | |
| | SLSLGK | |
| ACE2(ECD615)-IgG1 | METDTLLLWVLLLWVPGSTGQSTIEEQAKTFLDKFNHEAEDLFYQS | 47 |
| | SLASWNYNTNITEENVQNMNNAGDKWSAFLKEQSTLAQMYPLQEIQ | |
| | NLTVKLQLQALQQNGSSVLSEDKSKRLNTILNTMSTIYSTGKVCNP | |
| | DNPQECLLLEPGLNEIMANSLDYNERLWAWESWRSEVGKQLRPLYE | |
| | EYVVLKNEMARANHYEDYGDYWRGDYEVNGVDGYDYSRGQLIEDVE | |
| | HTFEEIKPLYEHLHAYVRAKLMNAYPSYISPIGCLPAHLLGDMWGR | |
| | FWTNLYSLTVPFGQKPNIDVTDAMVDQAWDAQRIFKEAEKFFVSVG | |
| | LPNMTQGFWENSMLTDPGNVQKAVCHPTAWDLGKGDFRILMCTKVT | |
| | MDDFLTAHHEMGHIQYDMAYAAQPFLLRNGANEGFHEAVGEIMSLS | |
| | AATPKHLKSIGLLSPDFQEDNETEINFLLKQALTIVGTLPFTYMLE | |
| | KWRWMVFKGEIPKDQWMKKWWEMKREIVGVVEPVPHDETYCDPASL | |
| | FHVSNDYSFIRYYTRTLYQFQFQEALCQAAKHEGPLHKCDISNSTE | |
| | AGQKLFNMLRLGKSEPWTLALENVVGAKNMNVRPLLNYFEPLFTWL | |
| | KDQNKNSFVGWSTDWSPYADEPKSCDKTHTCPPCPAPELLGGPSVF | |
| | LFPPKPKDTLMISRTPEVTCVVVDVSHEDPEVKFNWYVDGVEVHNA | |
| | KTKPREEQYNSTYRVVSVLTVLHQDWLNGKEYKCKVSNKALPAPIE | |
| | KTISKAKGQPREPQVYTLPPSREEMTKNQVSLTCLVKGFYPSDIAV | |
| | EWESNGQPENNYKTTPPVLDSDGSFFLYSKLTVDKSRWQQGNVFSC | |
| | SVMHEALHNHYTQKSLSLSPGK | |
| ACE2(ECD615)-IgG2 | METDTLLLWVLLLWVPGSTGQSTIEEQAKTFLDKFNHEAEDLFYQS | 48 |
| | SLASWNYNTNITEENVQNMNNAGDKWSAFLKEQSTLAQMYPLQEIQ | |
| | NLTVKLQLQALQQNGSSVLSEDKSKRLNTILNTMSTIYSTGKVCNP | |
| | DNPQECLLLEPGLNEIMANSLDYNERLWAWESWRSEVGKQLRPLYE | |
| | EYVVLKNEMARANHYEDYGDYWRGDYEVNGVDGYDYSRGQLIEDVE | |
| | HTFEEIKPLYEHLHAYVRAKLMNAYPSYISPIGCLPAHLLGDMWGR | |
| | FWTNLYSLTVPFGQKPNIDVTDAMVDQAWDAQRIFKEAEKFFVSVG | |
| | LPNMTQGFWENSMLTDPGNVQKAVCHPTAWDLGKGDFRILMCTKVT | |
| | MDDFLTAHHEMGHIQYDMAYAAQPFLLRNGANEGFHEAVGEIMSLS | |
| | AATPKHLKSIGLLSPDFQEDNETEINFLLKQALTIVGTLPFTYMLE | |
| | KWRWMVFKGEIPKDQWMKKWWEMKREIVGVVEPVPHDETYCDPASL | |
| | FHVSNDYSFIRYYTRTLYQFQFQEALCQAAKHEGPLHKCDISNSTE | |
| | AGQKLFNMLRLGKSEPWTLALENVVGAKNMNVRPLLNYFEPLFTWL | |
| | KDQNKNSFVGWSTDWSPYADERKCCVECPPCPAPPVAGPSVFLFPP | |
| | KPKDTLMISRTPEVTCVVVDVSHEDPEVQFNWYVDGVEVHNAKTKP | |
| | REEQFNSTFRVVSVLTVVHQDWLNGKEYKCKVSNKGLPAPIEKTIS | |
| | KTKGQPREPQVYTLPPSREEMTKNQVSLTCLVKGFYPSDISVEWES | |
| | NGQPENNYKTTPPMLDSDGSFFLYSKLTVDKSRWQQGNVFSCSVMH | |
| | EALHNHYTQKSLSLSPGK | |
| ACE2(ECD615)-IgG3 | METDTLLLWVLLLWVPGSTGQSTIEEQAKTFLDKFNHEAEDLFYQS | 49 |
| | SLASWNYNTNITEENVQNMNNAGDKWSAFLKEQSTLAQMYPLQEIQ | |
| | NLTVKLQLQALQQNGSSVLSEDKSKRLNTILNTMSTIYSTGKVCNP | |
| | DNPQECLLLEPGLNEIMANSLDYNERLWAWESWRSEVGKQLRPLYE | |
| | EYVVLKNEMARANHYEDYGDYWRGDYEVNGVDGYDYSRGQLIEDVE | |
| | HTFEEIKPLYEHLHAYVRAKLMNAYPSYISPIGCLPAHLLGDMWGR | |
| | FWTNLYSLTVPFGQKPNIDVTDAMVDQAWDAQRIFKEAEKFFVSVG | |
| | LPNMTQGFWENSMLTDPGNVQKAVCHPTAWDLGKGDFRILMCTKVT | |
| | MDDFLTAHHEMGHIQYDMAYAAQPFLLRNGANEGFHEAVGEIMSLS | |
| | AATPKHLKSIGLLSPDFQEDNETEINFLLKQALTIVGTLPFTYMLE | |
| | KWRWMVFKGEIPKDQWMKKWWEMKREIVGVVEPVPHDETYCDPASL | |
| | FHVSNDYSFIRYYTRTLYQFQFQEALCQAAKHEGPLHKCDISNSTE | |
| | AGQKLFNMLRLGKSEPWTLALENVVGAKNMNVRPLLNYFEPLFTWL | |
| | KDQNKNSFVGWSTDWSPYADELKTPLGDTTHTCPRCPEPKSCDTPP | |
| | PCPRCPEPKSCDTPPPCPRCPEPKSCDTPPPCPRCPAPELLGGPSV | |
| | FLFPPKPKDTLMISRTPEVTCVVVDVSHEDPEVQFKWYVDGVEVHN | |
| | AKTKPREEQYNSTFRVVSVLTVLHQDWLNGKEYKCKVSNKALPAPI | |
| | EKTISKTKGQPREPQVYTLPPSREEMTKNQVSLTCLVKGFYPSDIA | |
| | VEWESSGQPENNYNTTPPMLDSDGSFFLYSKLTVDKSRWQQGNIFS | |
| | CSVMHEALHNRFTQKSLSLSPGK | |
| ACE2(ECD615)-IgG4 | METDTLLLWVLLLWVPGSTGQSTIEEQAKTFLDKFNHEAEDLFYQS | 50 |
| | SLASWNYNTNITEENVQNMNNAGDKWSAFLKEQSTLAQMYPLQEIQ | |
| | NLTVKLQLQALQQNGSSVLSEDKSKRLNTILNTMSTIYSTGKVCNP | |
| | DNPQECLLLEPGLNEIMANSLDYNERLWAWESWRSEVGKQLRPLYE | |
| | EYVVLKNEMARANHYEDYGDYWRGDYEVNGVDGYDYSRGQLIEDVE | |
| | HTFEEIKPLYEHLHAYVRAKLMNAYPSYISPIGCLPAHLLGDMWGR | |
| | FWTNLYSLTVPFGQKPNIDVTDAMVDQAWDAQRIFKEAEKFFVSVG | |
| | LPNMTQGFWENSMLTDPGNVQKAVCHPTAWDLGKGDFRILMCTKVT | |
| | MDDFLTAHHEMGHIQYDMAYAAQPFLLRNGANEGFHEAVGEIMSLS | |
| | AATPKHLKSIGLLSPDFQEDNETEINFLLKQALTIVGTLPFTYMLE | |
| | KWRWMVFKGEIPKDQWMKKWWEMKREIVGVVEPVPHDETYCDPASL | |
| | FHVSNDYSFIRYYTRTLYQFQFQEALCQAAKHEGPLHKCDISNSTE | |
| | AGQKLFNMLRLGKSEPWTLALENVVGAKNMNVRPLLNYFEPLFTWL | |
| | KDQNKNSFVGWSTDWSPYADESKYGPPCPSCPAPEFLGGPSVFLFP | |
| | PKPKDTLMISRTPEVTCVVVDVSQEDPEVQFNWYVDGVEVHNAKTK | |
| | PREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWE | |
| | SNGQPENNYKTTPPVLDSDGSFFLYSRLTVDKSRWQEGNVFSCSVM | |
| | HEALHNHYTQKSLSLSLGK | |
| ACE2(ECD)-IgG1 | QSTIEEQAKTFLDKFNHEAEDLFYQSSLASWNYNTNITEENVQNMN | 52 |
| No signal peptide | NAGDKWSAFLKEQSTLAQMYPLQEIQNLTVKLQLQALQQNGSSVLS | |
| | EDKSKRLNTILNTMSTIYSTGKVCNPDNPQECLLLEPGLNEIMANS | |
| | LDYNERLWAWESWRSEVGKQLRPLYEEYVVLKNEMARANHYEDYGD | |
| | YWRGDYEVNGVDGYDYSRGQLIEDVEHTFEEIKPLYEHLHAYVRAK | |
| | LMNAYPSYISPIGCLPAHLLGDMWGRFWTNLYSLTVPFGQKPNIDV | |
| | TDAMVDQAWDAQRIFKEAEKFFVSVGLPNMTQGFWENSMLTDPGNV | |
| | QKAVCHPTAWDLGKGDFRILMCTKVTMDDFLTAHHEMGHIQYDMAY | |
| | AAQPFLLRNGANEGFHEAVGEIMSLSAATPKHLKSIGLLSPDFQED | |
| | NETEINFLLKQALTIVGTLPFTYMLEKWRWMVFKGEIPKDQWMKKW | |
| | WEMKREIVGVVEPVPHDETYCDPASLFHVSNDYSFIRYYTRTLYQF | |
| | QFQEALCQAAKHEGPLHKCDISNSTEAGQKLFNMLRLGKSEPWTLA | |
| | LENVVGAKNMNVRPLLNYFEPLFTWLKDQNKNSFVGWSTDWSPYAD | |
| | QSIKVRISLKSALGDKAYEWNDNEMYLFRSSVAYAMRQYFLKVKNQ | |
| | MILFGEEDVRVANLKPRISFNFFVTAPKNVSDIIPRTEVEKAIRMS | |
| | RSRINDAFRLNDNSLEFLGIQPTLGPPNQPPVSEPKSCDKTHTCPP | |
| | CPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYK | |
| | CKVSNKALPAPIEKTISKAKGQPREPQVYTLPPSREEMTKNQVSLT | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| ACE2(ECD)-IgG2 | QSTIEEQAKTFLDKFNHEAEDLFYQSSLASWNYNTNITEENVQNMN | 53 |
| No signal peptide | NAGDKWSAFLKEQSTLAQMYPLQEIQNLTVKLQLQALQQNGSSVLS | |
| | EDKSKRLNTILNTMSTIYSTGKVCNPDNPQECLLLEPGLNEIMANS | |
| | LDYNERLWAWESWRSEVGKQLRPLYEEYVVLKNEMARANHYEDYGD | |
| | YWRGDYEVNGVDGYDYSRGQLIEDVEHTFEEIKPLYEHLHAYVRAK | |
| | LMNAYPSYISPIGCLPAHLLGDMWGRFWTNLYSLTVPFGQKPNIDV | |
| | TDAMVDQAWDAQRIFKEAEKFFVSVGLPNMTQGFWENSMLTDPGNV | |
| | QKAVCHPTAWDLGKGDFRILMCTKVTMDDFLTAHHEMGHIQYDMAY | |
| | AAQPFLLRNGANEGFHEAVGEIMSLSAATPKHLKSIGLLSPDFQED | |
| | NETEINFLLKQALTIVGTLPFTYMLEKWRWMVFKGEIPKDQWMKKW | |
| | WEMKREIVGVVEPVPHDETYCDPASLFHVSNDYSFIRYYTRTLYQF | |
| | QFQEALCQAAKHEGPLHKCDISNSTEAGQKLFNMLRLGKSEPWTLA | |
| | LENVVGAKNMNVRPLLNYFEPLFTWLKDQNKNSFVGWSTDWSPYAD | |
| | QSIKVRISLKSALGDKAYEWNDNEMYLFRSSVAYAMRQYFLKVKNQ | |
| | MILFGEEDVRVANLKPRISFNFFVTAPKNVSDIIPRTEVEKAIRMS | |
| | RSRINDAFRLNDNSLEFLGIQPTLGPPNQPPVSERKCCVECPPCPA | |
| | PPVAGPSVFLFPPKPKDTLMISRTPEVTCVVVDVSHEDPEVQFNWY | |
| | VDGVEVHNAKTKPREEQFNSTFRVVSVLTVVHQDWLNGKEYKCKVS | |
| | NKGLPAPIEKTISKTKGQPREPQVYTLPPSREEMTKNQVSLTCLVK | |
| | GFYPSDISVEWESNGQPENNYKTTPPMLDSDGSFFLYSKLTVDKSR | |
| | WQQGNVFSCSVMHEALHNHYTQKSLSLSPGK | |
| ACE2(ECD)-IgG3 | QSTIEEQAKTFLDKFNHEAEDLFYQSSLASWNYNTNITEENVQNMN | 54 |
| No signal peptide | NAGDKWSAFLKEQSTLAQMYPLQEIQNLTVKLQLQALQQNGSSVLS | |
| | EDKSKRLNTILNTMSTIYSTGKVCNPDNPQECLLLEPGLNEIMANS | |
| | LDYNERLWAWESWRSEVGKQLRPLYEEYVVLKNEMARANHYEDYGD | |
| | YWRGDYEVNGVDGYDYSRGQLIEDVEHTFEEIKPLYEHLHAYVRAK | |
| | LMNAYPSYISPIGCLPAHLLGDMWGRFWTNLYSLTVPFGQKPNIDV | |
| | TDAMVDQAWDAQRIFKEAEKFFVSVGLPNMTQGFWENSMLTDPGNV | |
| | QKAVCHPTAWDLGKGDFRILMCTKVTMDDFLTAHHEMGHIQYDMAY | |
| | AAQPFLLRNGANEGFHEAVGEIMSLSAATPKHLKSIGLLSPDFQED | |
| | NETEINFLLKQALTIVGTLPFTYMLEKWRWMVFKGEIPKDQWMKKW | |
| | WEMKREIVGVVEPVPHDETYCDPASLFHVSNDYSFIRYYTRTLYQF | |
| | QFQEALCQAAKHEGPLHKCDISNSTEAGQKLFNMLRLGKSEPWTLA | |
| | LENVVGAKNMNVRPLLNYFEPLFTWLKDQNKNSFVGWSTDWSPYAD | |
| | QSIKVRISLKSALGDKAYEWNDNEMYLFRSSVAYAMRQYFLKVKNQ | |
| | MILFGEEDVRVANLKPRISFNFFVTAPKNVSDIIPRTEVEKAIRMS | |
| | RSRINDAFRLNDNSLEFLGIQPTLGPPNQPPVSELKTPLGDTTHTC | |
| | PRCPEPKSCDTPPPCPRCPEPKSCDTPPPCPRCPEPKSCDTPPPCP | |
| | RCPAPELLGGPSVFLFPPKPKDTLMISRTPEVTCVVVDVSHEDPEV | |
| | QFKWYVDGVEVHNAKTKPREEQYNSTFRVVSVLTVLHQDWLNGKEY | |
| | KCKVSNKALPAPIEKTISKTKGQPREPQVYTLPPSREEMTKNQVSL | |
| | TCLVKGFYPSDIAVEWESSGQPENNYNTTPPMLDSDGSFFLYSKLT | |
| | VDKSRWQQGNIFSCSVMHEALHNRFTQKSLSLSPGK | |
| ACE2(ECD)-IgG4 | QSTIEEQAKTFLDKFNHEAEDLFYQSSLASWNYNTNITEENVQNMN | 55 |
| No signal peptide | NAGDKWSAFLKEQSTLAQMYPLQEIQNLTVKLQLQALQQNGSSVLS | |
| | EDKSKRLNTILNTMSTIYSTGKVCNPDNPQECLLLEPGLNEIMANS | |
| | LDYNERLWAWESWRSEVGKQLRPLYEEYVVLKNEMARANHYEDYGD | |
| | YWRGDYEVNGVDGYDYSRGQLIEDVEHTFEEIKPLYEHLHAYVRAK | |
| | LMNAYPSYISPIGCLPAHLLGDMWGRFWTNLYSLTVPFGQKPNIDV | |
| | TDAMVDQAWDAQRIFKEAEKFFVSVGLPNMTQGFWENSMLTDPGNV | |
| | QKAVCHPTAWDLGKGDFRILMCTKVTMDDFLTAHHEMGHIQYDMAY | |
| | AAQPFLLRNGANEGFHEAVGEIMSLSAATPKHLKSIGLLSPDFQED | |
| | NETEINFLLKQALTIVGTLPFTYMLEKWRWMVFKGEIPKDQWMKKW | |
| | WEMKREIVGVVEPVPHDETYCDPASLFHVSNDYSFIRYYTRTLYQF | |
| | QFQEALCQAAKHEGPLHKCDISNSTEAGQKLFNMLRLGKSEPWTLA | |
| | LENVVGAKNMNVRPLLNYFEPLFTWLKDQNKNSFVGWSTDWSPYAD | |
| | QSIKVRISLKSALGDKAYEWNDNEMYLFRSSVAYAMRQYFLKVKNQ | |
| | MILFGEEDVRVANLKPRISFNFFVTAPKNVSDIIPRTEVEKAIRMS | |
| | RSRINDAFRLNDNSLEFLGIQPTLGPPNQPPVSESKYGPPCPSCPA | |
| | PEFLGGPSVFLFPPKPKDTLMISRTPEVTCVVVDVSQEDPEVQFNW | |
| | YVDGVEVHNAKTKPREEQFNSTYRVVSVLTVLHQDWLNGKEYKCKV | |
| | SNKGLPSSIEKTISKAKGQPREPQVYTLPPSQEEMTKNQVSLTCLV | |
| | KGFYPSDIAVEWESNGQPENNYKTTPPVLDSDGSFFLYSRLTVDKS | |
| | RWQEGNVFSCSVMHEALHNHYTQKSLSLSLGK | |
| ACE2(ECD615)-IgG1 | QSTIEEQAKTFLDKFNHEAEDLFYQSSLASWNYNTNITEENVQNMN | 56 |
| No signal peptide | NAGDKWSAFLKEQSTLAQMYPLQEIQNLTVKLQLQALQQNGSSVLS | |
| | EDKSKRLNTILNTMSTIYSTGKVCNPDNPQECLLLEPGLNEIMANS | |
| | LDYNERLWAWESWRSEVGKQLRPLYEEYVVLKNEMARANHYEDYGD | |
| | YWRGDYEVNGVDGYDYSRGQLIEDVEHTFEEIKPLYEHLHAYVRAK | |
| | LMNAYPSYISPIGCLPAHLLGDMWGRFWTNLYSLTVPFGQKPNIDV | |
| | TDAMVDQAWDAQRIFKEAEKFFVSVGLPNMTQGFWENSMLTDPGNV | |
| | QKAVCHPTAWDLGKGDFRILMCTKVTMDDFLTAHHEMGHIQYDMAY | |
| | AAQPFLLRNGANEGFHEAVGEIMSLSAATPKHLKSIGLLSPDFQED | |
| | NETEINFLLKQALTIVGTLPFTYMLEKWRWMVFKGEIPKDQWMKKW | |
| | WEMKREIVGVVEPVPHDETYCDPASLFHVSNDYSFIRYYTRTLYQF | |
| | QFQEALCQAAKHEGPLHKCDISNSTEAGQKLFNMLRLGKSEPWTLA | |
| | LENVVGAKNMNVRPLLNYFEPLFTWLKDQNKNSFVGWSTDWSPYAD | |
| | EPKSCDKTHTCPPCPAPELLGGPSVFLFPPKPKDTLMISRTPEVTC | |
| | VVVDVSHEDPEVKFNWYVDGVEVHNAKTKPREEQYNSTYRVVSVLT | |
| | VLHQDWLNGKEYKCKVSNKALPAPIEKTISKAKGQPREPQVYTLPP | |
| | SREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSP | |
| | GK | |
| ACE2(ECD615)-IgG2 | QSTIEEQAKTFLDKFNHEAEDLFYQSSLASWNYNTNITEENVQNMN | 57 |
| No signal peptide | NAGDKWSAFLKEQSTLAQMYPLQEIQNLTVKLQLQALQQNGSSVLS | |
| | EDKSKRLNTILNTMSTIYSTGKVCNPDNPQECLLLEPGLNEIMANS | |
| | LDYNERLWAWESWRSEVGKQLRPLYEEYVVLKNEMARANHYEDYGD | |
| | YWRGDYEVNGVDGYDYSRGQLIEDVEHTFEEIKPLYEHLHAYVRAK | |
| | LMNAYPSYISPIGCLPAHLLGDMWGRFWTNLYSLTVPFGQKPNIDV | |
| | TDAMVDQAWDAQRIFKEAEKFFVSVGLPNMTQGFWENSMLTDPGNV | |
| | QKAVCHPTAWDLGKGDFRILMCTKVTMDDFLTAHHEMGHIQYDMAY | |
| | AAQPFLLRNGANEGFHEAVGEIMSLSAATPKHLKSIGLLSPDFQED | |
| | NETEINFLLKQALTIVGTLPFTYMLEKWRWMVFKGEIPKDQWMKKW | |
| | WEMKREIVGVVEPVPHDETYCDPASLFHVSNDYSFIRYYTRTLYQF | |
| | QFQEALCQAAKHEGPLHKCDISNSTEAGQKLFNMLRLGKSEPWTLA | |
| | LENVVGAKNMNVRPLLNYFEPLFTWLKDQNKNSFVGWSTDWSPYAD | |
| | ERKCCVECPPCPAPPVAGPSVFLFPPKPKDTLMISRTPEVTCVVVD | |
| | VSHEDPEVQFNWYVDGVEVHNAKTKPREEQFNSTFRVVSVLTVVHQ | |
| | DWLNGKEYKCKVSNKGLPAPIEKTISKTKGQPREPQVYTLPPSREE | |
| | MTKNQVSLTCLVKGFYPSDISVEWESNGQPENNYKTTPPMLDSDGS | |
| | FFLYSKLTVDKSRWQQGNVFSCSVMHEALHNHYTQKSLSLSPGK | |
| ACE2(ECD615)-IgG3 | QSTIEEQAKTFLDKFNHEAEDLFYQSSLASWNYNTNITEENVQNMN | 58 |
| No signal peptide | NAGDKWSAFLKEQSTLAQMYPLQEIQNLTVKLQLQALQQNGSSVLS | |
| | EDKSKRLNTILNTMSTIYSTGKVCNPDNPQECLLLEPGLNEIMANS | |
| | LDYNERLWAWESWRSEVGKQLRPLYEEYVVLKNEMARANHYEDYGD | |
| | YWRGDYEVNGVDGYDYSRGQLIEDVEHTFEEIKPLYEHLHAYVRAK | |
| | LMNAYPSYISPIGCLPAHLLGDMWGRFWTNLYSLTVPFGQKPNIDV | |
| | TDAMVDQAWDAQRIFKEAEKFFVSVGLPNMTQGFWENSMLTDPGNV | |
| | QKAVCHPTAWDLGKGDFRILMCTKVTMDDFLTAHHEMGHIQYDMAY | |
| | AAQPFLLRNGANEGFHEAVGEIMSLSAATPKHLKSIGLLSPDFQED | |
| | NETEINFLLKQALTIVGTLPFTYMLEKWRWMVFKGEIPKDQWMKKW | |
| | WEMKREIVGVVEPVPHDETYCDPASLFHVSNDYSFIRYYTRTLYQF | |
| | QFQEALCQAAKHEGPLHKCDISNSTEAGQKLFNMLRLGKSEPWTLA | |
| | LENVVGAKNMNVRPLLNYFEPLFTWLKDQNKNSFVGWSTDWSPYAD | |
| | ELKTPLGDTTHTCPRCPEPKSCDTPPPCPRCPEPKSCDTPPPCPRC | |
| | PEPKSCDTPPPCPRCPAPELLGGPSVFLFPPKPKDTLMISRTPEVT | |
| | CVVVDVSHEDPEVQFKWYVDGVEVHNAKTKPREEQYNSTFRVVSVL | |
| | TVLHQDWLNGKEYKCKVSNKALPAPIEKTISKTKGQPREPQVYTLP | |
| | PSREEMTKNQVSLTCLVKGFYPSDIAVEWESSGQPENNYNTTPPML | |
| | DSDGSFFLYSKLTVDKSRWQQGNIFSCSVMHEALHNRFTQKSLSLS | |
| | PGK | |
| ACE2(ECD615)-IgG4 | QSTIEEQAKTFLDKFNHEAEDLFYQSSLASWNYNTNITEENVQNMN | 59 |
| No signal peptide | NAGDKWSAFLKEQSTLAQMYPLQEIQNLTVKLQLQALQQNGSSVLS | |
| GL-4316-mature | EDKSKRLNTILNTMSTIYSTGKVCNPDNPQECLLLEPGLNEIMANS | |
| | LDYNERLWAWESWRSEVGKQLRPLYEEYVVLKNEMARANHYEDYGD | |
| | YWRGDYEVNGVDGYDYSRGQLIEDVEHTFEEIKPLYEHLHAYVRAK | |
| | LMNAYPSYISPIGCLPAHLLGDMWGRFWTNLYSLTVPFGQKPNIDV | |
| | TDAMVDQAWDAQRIFKEAEKFFVSVGLPNMTQGFWENSMLTDPGNV | |
| | QKAVCHPTAWDLGKGDFRILMCTKVTMDDFLTAHHEMGHIQYDMAY | |
| | AAQPFLLRNGANEGFHEAVGEIMSLSAATPKHLKSIGLLSPDFQED | |
| | NETEINFLLKQALTIVGTLPFTYMLEKWRWMVFKGEIPKDQWMKKW | |
| | WEMKREIVGVVEPVPHDETYCDPASLFHVSNDYSFIRYYTRTLYQF | |
| | QFQEALCQAAKHEGPLHKCDISNSTEAGQKLFNMLRLGKSEPWTLA | |
| | LENVVGAKNMNVRPLLNYFEPLFTWLKDQNKNSFVGWSTDWSPYAD | |
| | ESKYGPPCPSCPAPEFLGGPSVFLFPPKPKDTLMISRTPEVTCVVV | |
| | DVSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |

In some embodiments, the ACE2-Fc fusion protein of the present disclosure comprises an amino acid sequence that is at least 80%, 85%, 90%, 95%, 96%, 97%, 98%, or 99% identical to SEQ ID NO: 43. In some embodiments, the ACE2-Fc fusion protein of the present disclosure comprises or consists of SEQ ID NO: 43.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 44. In some embodiments, the ACE2-Fc fusion protein of the present disclosure comprises or consists of SEQ ID NO: 44.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 45. In some embodiments, the ACE2-Fc fusion protein of the present disclosure comprises or consists of SEQ ID NO: 45.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 46. In some embodiments, the ACE2-Fc fusion protein of the present disclosure comprises or consists of SEQ ID NO: 46.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 47. In some embodiments, the ACE2-Fc fusion protein of the present disclosure comprises or consists of SEQ ID NO: 47.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 48. In some embodiments, the ACE2-Fc fusion protein of the present disclosure comprises or consists of SEQ ID NO: 48.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 49. In some embodiments, the ACE2-Fc fusion protein of the present disclosure comprises or consists of SEQ ID NO: 49.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 50. In some embodiments, the ACE2-Fc fusion protein of the present disclosure comprises or consists of SEQ ID NO: 50.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 52. In some embodiments, the ACE2-Fc fusion protein of the present disclosure comprises or consists of SEQ ID NO: 52.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 53. In some embodiments, the ACE2-Fc fusion protein of the present disclosure comprises or consists of SEQ ID NO: 53.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 54. In some embodiments, the ACE2-Fc fusion protein of the present disclosure comprises or consists of SEQ ID NO: 54.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 55. In some embodiments, the ACE2-Fc fusion protein of the present disclosure comprises or consists of SEQ ID NO: 55.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 56. In some embodiments, the ACE2-Fc fusion protein of the present disclosure comprises or consists of SEQ ID NO: 56.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 57. In some embodiments, the ACE2-Fc fusion protein of the present disclosure comprises or consists of SEQ ID NO: 57.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 58. In some embodiments, the ACE2-Fc fusion protein of the present disclosure comprises or consists of SEQ ID NO: 58.

In some embodiments, the ACE2-Fc fusion protein of the present disclosure comprises an amino acid sequence that is at least 80%, 85%, 90%, 95%, 96%, 97%, 98%, or 99% identical to SEQ ID NO: 59. In some embodiments, the ACE2-Fc fusion protein of the present disclosure comprises or consists of SEQ ID NO: 59.

In some embodiments, the ACE2-Fc fusion protein of the present disclosure comprises an ACE2 extracellular domain or fragment thereof comprising an amino acid sequence of SEQ ID NO: 9; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises: (i) a signal peptide comprising an amino acid sequence of SEQ ID NO: 1; (ii) an ACE2 extracellular domain or fragment thereof comprising an amino acid sequence of SEQ ID NO: 10; and an IgG1 Fc domain comprising an amino acid sequence of SEQ ID NO: 39. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 11; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 12; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 13; and an IgG1 Fc domain comprising an amino acid sequence of SEQ ID NO: 39. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 14; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 15; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 16; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 17; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 18; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 19; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 20; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 21; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 22; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 23; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 24; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 25; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 26; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 27; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 28; and an IgG1 Fc domain comprising an amino acid sequence of SEQ ID NO: 39. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 29; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 30; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 31; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 32; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 33; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 34; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 35; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 36; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 37; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 38; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 51; and an IgG1 Fc domain comprising an amino acid sequence of SEQ ID NO: 39. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 9; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 10; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 11; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 12; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 13; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 14; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 15; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 16; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 17; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 18; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 19; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 20; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 21; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 22; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 23; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 24; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 25; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 26; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 27; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 28; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 29; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 30; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 31; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 32; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 33; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 34; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 35; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 36; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 37; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 38; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion protein of the present disclosure comprises an ACE2 extracellular domain or fragment thereof comprising an amino acid sequence of SEQ ID NO: 51; and an IgG4 Fc domain comprising an amino acid sequence of SEQ ID NO: 42. In some embodiments, the ACE2-Fc fusion protein further comprises a signal peptide comprising an amino acid sequence of SEQ ID NO: 1. In some embodiments, the signal peptide comprising the amino acid sequence of SEQ ID NO: 1 is cleaved from the ACE2-Fc fusion protein.

In some embodiments, the ACE2-Fc fusion proteins described herein function as a decoy receptor. The term “decoy receptor” as used herein refers to a protein that binds to a pathogenic microorganism and inhibits entry and/or replication of the pathogenic microorganism in host cells. In some embodiments, the ACE2-Fc fusion protein binds to the pathogenic microorganism with avidity due to one or more ACE2 extracellular domains or fragments thereof. In some embodiments, the avid binding of the ACE2-Fc fusion protein to the pathogenic microorganism neutralizes the pathogenic microorganism. In some embodiments, the avid binding of the ACE2-Fc fusion protein to the pathogenic microorganism is associated with diminished pathologic effects in an infected subject.

In some embodiments, the ACE2-Fc fusion protein binds to one or more viral spike proteins on the surface of a coronavirus, such as SARS-CoV-1 or SARS-CoV-2, and inhibits or prevents viral entry and/or replication in host cells. In some embodiments, the ACE2-Fc fusion protein binds to the coronavirus with avidity due to one or more ACE2 extracellular domains or fragments thereof. In some embodiments, the avid binding of the ACE2-Fc fusion protein to the coronavirus spike protein neutralizes the coronavirus in an infected subject. In some embodiments, the avid binding of the ACE2-Fc fusion protein to coronavirus spike protein is associated with diminished pathologic effects in an infected subject. In some embodiments, the avid binding of the ACE2-Fc fusion protein to the coronavirus spike protein mimics the binding of viral spike protein to host cell ACE2 receptor. As a result, it is not possible for the virus to mutate away from the compounds of this invention without also losing potency in binding to host ACE2 and therefore becoming less infectious and causing less morbidity and mortality, unless the virus successfully switches to a different host receptor. Put differently, no matter how a virus such as SARS-CoV-2 mutates its spike protein to evade other selective pressures, the virus cannot evade binding by the ACE2-Fc fusion proteins described herein unless the virus becomes less virulent or ceases using ACE2 as the primary host cell receptor.

Antibody-dependent enhancement (ADE) is a well described feature of coronaviruses. ADE occurs when antibodies (e.g., antibodies against SARS-CoV-2) bind Fc receptors on host cells thereby facilitating viral entry (Lee et al, Nat. Microbiol 5; 2020). Thus, ADE can increase viral burden and cause more severe disease. In some embodiments, the ACE2-Fc fusion proteins described herein inhibit or decrease ADE of viral entry into host cells. In some embodiments, the ACE2-Fc fusion proteins comprising an IgG4 Fc domain (e.g., SEQ ID NOs: 46 or SEQ ID NO: 50) or a mutated IgG1 or IgG3 Fc domain (e.g., Leu234Ala and Leu235Ala (commonly called LALA mutations)) inhibit or decrease ADE of viral entry into host cells. See, Wan et al., Journal of Virology 2020; (94)5:e02015-19; Gralinski et al., mBio 2018; (9)5:e01753-18; and Mehlhop et al., Cell Host and Microbe 2007; 2:417-426, herein incorporated by reference in their entirety.

The ACE2 extracellular domain contains a dimerization domain which can lead to ACE2 aggregation or multimerization under certain circumstances. “Increased multimerization” as used herein refers to an increase in the percentage of multimers (e.g., dimers of the homodimer, trimers of the homodimer, tetramers of the homodimer, etc.) present after purification compared to the percentage of multimers of the parental ACE2-Fc fusion protein when cultured under the same conditions (e.g., media, cell type, temperature, culture time, etc.).

In some embodiments, the ACE2-Fc fusion proteins comprising one or more variants in the ACE2 extracellular domain or fragment thereof exhibit decreased multimerization relative to the corresponding parental ACE2-Fc fusion protein. “Decreased multimerization” as used herein refers to an decrease in the percentage of multimers (e.g., dimers of the homodimer, trimers of the homodimer, tetramers of the homodimer, etc.) present after purification compared to the percentage of multimers of the parental ACE2-Fc fusion protein when cultured under the same conditions (e.g., media, cell type, temperature, culture time, etc.).

In some embodiments, the ACE2-Fc fusion protein binds to a coronavirus. In some embodiments, the ACE2-Fc fusion protein binds to the coronavirus with Kd of about 1 nM to about 100 nM. In some embodiments, the ACE2-Fc fusion protein binds to the coronavirus with Kd of about 0.01 nM, about 0.1 nM, about 1 nM, about 5 nM, about 10 nM, about 20 nM, about 30 nM, about 40 nM, about 50 nM, about 60 nM, about 70 nM, about 80 nM, about 90 nM, about 100 nM, about 250 nM, or about 500 nM, about 1 nM, about 5000 nM, In some embodiments, the ACE2-Fc fusion protein binds to the coronavirus with Kd of about 1 μM, about 5 μM, about 10 μM, about 20 μM, about 30 μM, about 40 μM, about 50 μM, about 60 μM, about 70 μM, about 80 μM, about 90 μM, about 100 μM, about 250 μM, or about 500 μM, about 1 μM, or about 5000 μM.

In some embodiments, the ACE2-Fc fusion protein binds to a coronavirus spike protein. For example, in some embodiments, the ACE2-Fc fusion protein binds to a coronavirus spike protein of one of SEQ ID NOs: 60-64. In some embodiments, the ACE2-Fc fusion protein binds to the coronavirus spike protein with Kd of about 1 nM to about 100 nM. In some embodiments, the ACE2-Fc fusion protein binds to the coronavirus spike protein with Kd of about 0.01 nM, about 0.1 nM, about 1 nM, about 5 nM, about 10 nM, about 20 nM, about 30 nM, about 40 nM, about 50 nM, about 60 nM, about 70 nM, about 80 nM, about 90 nM, about 100 nM, about 250 nM, or about 500 nM, about 1 nM, about 5000 nM, In some embodiments, the ACE2-Fc fusion protein binds to the coronavirus spike protein with Kd of about 1 μM, about 5 μM, about 10 μM, about 20 μM, about 30 μM, about 40 μM, about 50 μM, about 60 μM, about 70 μM, about 80 μM, about 90 μM, about 100 μM, about 250 μM, or about 500 μM, about 1 μM, or about 5000 μM.

In some embodiments, the ACE2-Fc fusion protein binds to an ACE2 ligand. In some embodiments, the ACE2-Fc fusion protein binds to the ACE2 ligand with Kd of about 1 nM to about 100 nM. In some embodiments, the ACE2-Fc fusion protein binds to the ACE2 ligand with Kd of about 0.01 nM, about 0.1 nM, about 1 nM, about 5 nM, about 10 nM, about 20 nM, about 30 nM, about 40 nM, about 50 nM, about 60 nM, about 70 nM, about 80 nM, about 90 nM, about 100 nM, about 250 nM, or about 500 nM. In some embodiments, the ACE2-Fc fusion protein binds to the ACE2 ligand with Kd of about 1 μM, about 5 μM, about 10 μM, about 20 μM, about 30 μM, about 40 μM, about 50 μM, about 60 μM, about 70 μM, about 80 μM, about 90 μM, about 100 μM, about 250 μM, or about 500 μM, about 1 μM, or about 5000 μM. In some embodiments, the ACE2 ligand is selected from the group consisting of angiotensin I, angiotensin II, apelin, pro-dynorphin, or des-arg⁹-bradykinin.

In some embodiments, homodimers of the ACE2-Fc fusion protein bind to viral spike protein with increased avidity compared to multimers of the ACE2-Fc fusion proteins. In some embodiments, homodimers of the ACE2-Fc fusion proteins described herein bind viral spike protein with decreased avidity compared to multimers of the ACE2-Fc fusion protein.

In some embodiments, multimers of certain ACE2-Fc fusion proteins described herein bind to viral spike proteins with increased potency compared to homodimers of the ACE2-Fc fusion protein. In some embodiments, multimers of the ACE2-Fc fusion protein bind viral spike protein with decreased potency compared to homodimers of the ACE2-Fc fusion proteins described herein.

In some embodiments, the ACE2-Fc fusion protein comprising an IgG4 Fc domain exhibits increased binding to viral spike compared to an ACE2-Fc fusion protein comprising an IgG1 Fc domain. In some embodiments, the ACE2-Fc fusion protein comprising the IgG4 Fc domain is less constrained and more flexible compared with an otherwise identical ACE2-Fc fusion protein comprising an IgG1 domain, resulting in increased binding potency to viral spike protein.

In some embodiments, the ACE2-Fc fusion protein of the present disclosure undergoes Fab-arm exchange. The phrase “Fab-arm exchange” as used herein refers to an exchange of Fab-arms by swapping a heavy chain and attached light chain (half-molecule) with a heavy-light chain pair from another molecule, which results in bispecific antibodies. See, van der Neut Kolfschoten et al., Science, 2007; 317(5844):1554-7. In some embodiments, the ACE2-Fc fusion protein forms a bispecific antibody in vivo following administration of the ACE2-Fc fusion protein to a subject. In some embodiments, the bispecific antibody formed in vivo comprises an ACE2 arm and a Fab-arm. In some embodiments, the bispecific antibody formed in vivo comprises an ACE2 and an Fab-arm, and the Fab-arm targets the bispecific antibody to sites of infection and/or inflammation.

In some embodiments, the ACE2-Fc fusion protein comprising an IgG4 Fc domain comprises one or more mutations to decrease or eliminate Fab-arm exchange. In some embodiments, the one or more mutations in the IgG4 Fc domain of the ACE2-Fc fusion protein decreases Fab-arm exchange by about 10%, about 20%, about 30%, about 40%, about 50%, about 60%, about 70%, about 80%, about 90%, or about 100% compared to the parental ACE2-Fc fusion protein. In some embodiments, the ACE2-Fc fusion protein comprises a S228P mutation in the IgG4 Fc domain to decrease or eliminate Fab-arm exchange. In some embodiments, the ACE2-Fc fusion protein comprises a Y219C mutation in the IgG4 Fc domain to decrease or eliminate Fab-arm exchange. In some embodiments, the ACE2-Fc fusion protein comprises a G220C mutation in the IgG4 Fc domain to decrease or eliminate Fab-arm exchange. In some embodiments, the ACE2-Fc fusion protein comprises a S228P, Y219C, and/or G220C mutation in the IgG4 Fc domain to decrease or eliminate Fab-arm exchange. In some embodiments, the ACE2-Fc fusion protein comprises one or more mutations known to a skilled artisan to decrease or eliminate Fab-arm exchange. See, Silva et al., J Biol Chem, 2015; 290(9):5462-5469; and Handlogten et al., mAbs, 2020; 12, Article No. 1779974.

In some embodiments, the ACE2-Fc fusion proteins described herein exhibit reduced Fc-mediated effector function. “Reduced Fc-mediated effector function” as used herein refers to a decrease in binding to one or more low affinity Fcγ receptors (FcγRIIA, FcγRIIB, or FcγRIII), reduced complement binding (e.g., Clq), reduced phagocytosis, and/or reduced cellular cytotoxicity. In some embodiments, the ACE2-Fc fusion protein comprises one or more mutations in the Fc domain to reduce binding to Fcγ receptors, thereby preventing FcR binding and subsequent effector function.

In some embodiments, the ACE2-Fc fusion protein comprising an IgG4 Fc domain or mutated IgG1 or IgG3 Fc domain has reduced Fc-mediated effector function compared to an ACE2-Fc fusion protein comprising a wild type IgG1, IgG2, or IgG3 Fc domain. In some embodiments, the ACE2-Fc fusion protein comprising an IgG4 Fc domain or mutated IgG1 or IgG3 Fc domain exhibits reduced complement activation compared to an ACE2-Fc fusion protein comprising a wild type IgG1, IgG2, or IgG3 Fc domain. In some embodiments, the ACE2-Fc fusion protein comprising an IgG4 Fc domain or mutated IgG1 or IgG3 Fc domain exhibits reduced binding to complement Clq compared to an ACE2-Fc fusion protein comprising a wild type IgG1, IgG2, or IgG3 Fc domain. In some embodiments, the ACE2-Fc fusion protein comprising an IgG4 Fc domain or mutated IgG1 or IgG3 Fc domain exhibits reduced immune cell activation compared to an ACE2-Fc fusion protein comprising a wild type IgG1, IgG2, or IgG3 Fc domain. some embodiments, the ACE2-Fc fusion protein comprising an IgG4 Fc domain or mutated IgG1 or IgG3 Fc domain exhibits reduced binding to low affinity Fc gamma receptors (e.g., FcγRIIA, FcγRIIB, or FcγRIII) compared to an ACE2-Fc fusion protein comprising a wild type IgG1, IgG2, or IgG3 Fc domain. In some embodiments, the ACE2-Fc fusion protein comprising an IgG4 Fc domain or mutated IgG1 or IgG3 Fc domain exhibits reduced immune effector function compared to an ACE2-Fc fusion protein comprising a wild type IgG1, IgG2, or IgG3 Fc domain. In some embodiments, the ACE2-Fc fusion protein comprising an IgG4 Fc domain or mutated IgG1 or IgG3 Fc domain exhibits reduced antibody-dependent cellular cytotoxicity (ADCC), complement-dependent cytotoxicity (CDC), and/or antibody-dependent cellular phagocytosis (ADCP) compared to a wild type ACE2-Fc fusion protein comprising an IgG1, IgG2, or IgG3 Fc domain. See, van der Neut Kolfschoten et al., Science, 2007; 317(5844):1554-7.

In some embodiments, particular ACE2-Fc fusion proteins described herein exhibit increased Fc-mediated effector function. “Increased Fc-mediated effector function” as used herein refers to an increase in binding to one or more low affinity Fcγ receptors (e.g., FcγRIIA, FcγRIIB, or FcγRIII), increased complement binding (e.g., Clq), increased phagocytosis, and/or increased cellular cytotoxicity. In some embodiments, the ACE2-Fc fusion protein comprising one or more variants in the ACE2 extracellular domain exhibits increased function relative to the parental ACE2-Fc fusion protein. “Increased function” as used herein refers to an increase in one or more functions of the variant ACE2-Fc fusion protein, e.g., binding to virus (e.g., SARS-CoV-2), virus cell entry inhibition assay, efficacy in SARS-CoV-2 animal models, binding to ACE2 ligands (e.g., angiotensin II), and ACE2 enzymatic activity. In some embodiments, the ACE2-Fc fusion protein comprising one or more variants in the ACE2 extracellular domain or fragment thereof exhibits substantially the same or similar function relative to the parental ACE2-Fc fusion protein. In some embodiments, the ACE2-Fc fusion protein comprising one or more variants in the ACE2 extracellular domain or fragment thereof exhibits decreased function relative to the parental ACE2-Fc fusion protein. In such embodiments, the ACE2 variant proteins with decreased function relative to the parental ACE2-Fc fusion protein are capable of exerting a therapeutic effect, i.e., treating or preventing viral infection (e.g., SARS-CoV-2) or cardiovascular disease.

### Pharmaceutical Compositions

The compositions described herein may include pharmaceutical compositions formulated for administration to a subject in need thereof.

Administration of the ACE2-Fc fusion proteins described herein will be via any common route, orally, parenterally, or topically. Exemplary routes include, but are not limited to oral, nasal, inhaled, buccal, rectal, vaginal, ophthalmic, subcutaneous, intramuscular, intraperitoneal, intravenous, intraarterial, intratumoral, spinal, intrathecal, intra-articular, intra-arterial, sub-arachnoid, sublingual, oral mucosal, bronchial, lymphatic, intra-uterine, parenteral, subcutaneous, intratumor, integrated on an implantable device such as a suture or in an implantable device such as an implantable polymer, intradural, intracortical, or dermal. Such compositions would be administered as pharmaceutically acceptable compositions as described herein. The route of administration would depend on the nature of the disease being treated. In some embodiments, the ACE2-Fc fusion protein is administered intravenously, subcutaneously, or intramuscularly.

The term “parenteral administration” as used herein includes any form of administration in which the compound is absorbed into the subject without involving absorption via the intestines. Exemplary parenteral administrations that are used in the present invention include, but are not limited to intramuscular, intravenous, intraperitoneal, intratumoral, intraocular, nasal or intraarticular administration.

The term “intravenous administration” as used herein includes all techniques to deliver a compound or composition of the present invention to the systemic circulation via an intravenous injection or infusion.

The term “topical administration” as used herein includes application to a dermal, epidermal, subcutaneous or mucosal surface.

The phrase “pharmaceutically acceptable carrier” means a carrier or excipient that is useful in preparing a pharmaceutical composition that is generally safe and non-toxic, and includes an excipient that is acceptable for veterinary use as well as human pharmaceutical use.

Pharmaceutically acceptable carriers as used herein includes any and all solvents, dispersion media, coatings, antibacterial and antifungal agents, isotonic and absorption delaying agents and the like. The use of such media and agents for pharmaceutically active substances is well known in the art. Supplementary active ingredients can also be incorporated into the compositions described herein.

The ACE2-Fc fusion proteins described herein may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Sterile injectable solutions are prepared by incorporating the ACE2-Fc fusion proteins in the required amount of appropriate solvent with various other ingredients enumerated above, as required, followed by filtered sterilization. In some embodiments, the sterile injectable solutions are formulated for intramuscular, subcutaneous, or intravenous administration. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In some embodiments, the ACE2-Fc fusion proteins described herein are suitable for oral administration and provided in a pharmaceutically acceptable carrier with or without an inert diluent. The carrier should be assimilable or edible and includes liquid, semi-solid (e.g., pastes), or solid carriers. Examples of carriers or diluents include fats, oils, water, saline solutions, lipids, liposomes, resins, binders, fillers and the like, or combinations thereof. The term “oral administration” as used herein includes oral, buccal, enteral or intragastric administration.

In some embodiments, the ACE2-Fc fusion protein is combined with the carrier in any convenient and practical manner, i.e., by solution, suspension, emulsification, admixture, encapsulation, microencapsulation, absorption and the like. Such procedures are routine for those skilled in the art.

In some embodiments, the ACE2-Fc fusion protein is in powder form and combined or mixed thoroughly with a semi-solid or solid carrier. The mixing can be carried out in any convenient manner such as grinding. Stabilizing agents can be also added in the mixing process in order to protect the composition from loss of therapeutic activity through, i.e., denaturation in the stomach. Examples of stabilizers for use in an orally administrable composition include buffers, antagonists to the secretion of stomach acids, amino acids such as glycine and lysine, carbohydrates such as dextrose, mannose, galactose, fructose, lactose, sucrose, maltose, sorbitol, mannitol, etc., proteolytic enzyme inhibitors, and the like. More preferably, for an orally administered composition, the stabilizer can also include antagonists to the secretion of stomach acids. In some embodiments, the ACE2-Fc fusion protein is a dry powder for inhalation.

The ACE2-Fc fusion protein that is combined with a semi-solid or solid carrier can be further formulated into hard or soft shell gelatin capsules, tablets, or pills. In some embodiments, the gelatin capsules, tablets, or pills are enterically coated. Enteric coatings prevent denaturation of the composition in the stomach or upper bowel where the pH is acidic. See, e.g., U.S. Pat. No. 5,629,001. Upon reaching the small intestines, the basic pH dissolves the coating and permits the composition to be released.

In some embodiments, the ACE2-Fc fusion protein in powder form is combined or mixed thoroughly with materials that create a nanoparticle encapsulating the ACE2-Fc fusion protein or to which the ACE2-Fc fusion protein is attached. Each nanoparticle will have a size of less than or equal to 100 microns. The nanoparticle may have mucoadhesive properties that allow for gastrointestinal absorption of the ACE2-Fc fusion protein that would otherwise not be orally bioavailable.

In some embodiments, the ACE2-Fc fusion protein in powder form is combined with a liquid carrier such as, i.e., water or a saline solution, with or without a stabilizing agent.

In some embodiments, the ACE2-Fc fusion protein formulation is a solution in a hypotonic phosphate based buffer that is free of potassium. This formulation may be administered via any route of administration, for example, but not limited to intravenous administration.

In some embodiments, the ACE2-Fc fusion proteins described herein are suitable for topical administration. In some embodiments, the ACE2-Fc fusion proteins comprising a semi-solid carrier can be further formulated into a cream or gel ointment. A preferred carrier for the formation of a gel ointment is a gel polymer. Examples of polymers that are used in the formulation of a gel composition include, but are not limited to, carbopol, carboxymethyl-cellulose, and pluronic polymers.

Further, the ACE2-Fc fusion proteins of the present disclosure can be formulated into a polymer for subcutaneous or subdermal implantation. A preferred formulation for the implantable drug-infused polymer is an agent Generally Regarded as Safe and may include, for example, cross-linked dextran, dextran-tyramine, dextran-polyethylene glycol, or dextran-gluteraldehyde. Implantable drug-infused polymers are further described in Samantha Hart, Master of Science Thesis, “Elution of Antibiotics from a Novel Cross-Linked Dextran Gel: Quantification” Virginia Polytechnic Institute and State University, Jun. 8, 2009; Jin, et al. (2010) Tissue Eng. Part A. 16(8):2429-40; Jukes, et al. (2010) Tissue Eng. Part A., 16(2):565-73; and Brondsted, et al. (1998) J. Controlled Release, 53:7-13. One skilled in the art will know that many similar polymers and hydrogels can be formed incorporating the ACE2-Fc fusion protein fixed within the polymer or hydrogel and controlling the pore size to the desired diameter.

In some embodiments, the ACE2-Fc fusion proteins are formulated for administration to the eye, such as by eye drop or balm.

Upon formulation, the ACE2-Fc fusion proteins described herein are administered in a manner compatible with the dosage formulation and in such amount as is therapeutically effective to result in an improvement or remediation of the symptoms. The formulations are easily administered in a variety of dosage forms such as ingestible solutions, drug release capsules and the like. Some variation in dosage can occur depending on the condition of the subject being treated. The person responsible for administration can, in any event, determine the appropriate dose for the individual subject. Moreover, for human administration, preparations meet sterility, general safety and purity standards as required by FDA Center for Biologics Evaluation and Research standards.

Recombinant human ACE2 has a half-life of about 8 hours with a terminal half-life of about 12 hours in humans. In some embodiments, the ACE2-Fc fusion protein or pharmaceutical composition thereof has a longer half-life relative to recombinant human ACE2 in a subject. In some embodiments, the ACE2-Fc fusion protein or pharmaceutical composition thereof has a longer half-life relative to recombinant human ACE2 by about 4 hours, about 8 hours, about 12 hours, about 24 hours, about 48 hours, about 72 hours, about 96 hours, about 1 week, about 2 weeks, or about 1 month in a subject. In some embodiments, the ACE2-Fc fusion protein or pharmaceutical composition thereof has a half-life of about 12 hours, about 24 hours, about 36 hours, about 48 hours, about 72 hours, about 96 hours, about 1 week, about 2 weeks, or about 1 month in a subject.

### Methods of Treatment

In some embodiments, the disclosure relates to methods of treating or preventing a disease or disorder in a subject in need thereof. In some embodiments, the method of treating or preventing a disease or disorder in a subject comprises administering to the subject an ACE2-Fc fusion protein that diminishes or reduces the effects of a virus such as coronavirus (e.g., SARS-CoV-1 or SARS-CoV-2) or any other microorganism (e.g., virus or bacteria) that directly uses the ACE2 receptor for pathogenesis or which indirectly decreases available ACE2 enzymatic function (e.g. pandemic flu). In some embodiments, the method for treating or preventing a disease or disorder in a subject comprises administering to the subject an ACE2-Fc fusion protein that binds to and/or inhibits the activity of a coronavirus, (e.g., SARS-CoV-1 or SARS-CoV-2). In some embodiments, the method for treating or preventing a disease or disorder in a subject comprises administering to the subject an ACE2-Fc fusion protein that exhibits increased binding to coronavirus (e.g., SARS-CoV-1 or SARS-CoV-2). In some embodiments, the method for treating or preventing a disease or disorder in a subject comprises administering to the subject an ACE2-Fc fusion protein that is enzymatically active in a subject whose ACE2 is either absolutely decreased (e.g. SARS-CoV-2) or relatively insufficient as evidenced by increased angiotensin II (e.g. influenza-mediated or toxin-mediated acute lung injury, pulmonary artery hypertension).

In some embodiments, the present disclosure provides a method of treating a disease or disorder in a subject in need thereof comprising detecting a level of angiotensin II (Ang II), detecting a ratio of Ang II to Ang 1-7, or detecting a level of des-arg-9-bradykinin in the subject and administering the ACE2-Fc fusion protein to the subject if an elevated level of Ang II or des-arg-9-bradykinin, or an elevated Ang II/Ang 1-7 ratio is detected.

In some embodiments, the present disclosure provides methods of treating a coronavirus infection in a subject in need thereof comprising administering the ACE2-Fc fusion proteins provided herein. In some embodiments, the coronavirus is SARS-CoV-1 or SARS-CoV-2, or variants thereof. In some embodiments, the coronavirus is a SARS-CoV-2 variant. SARS-CoV-2 variants refer to viral strains comprising one or more amino acid mutations compared to the virus strain that originated in Wuhan China. Exemplary SARS-CoV-2 variants include SARS-CoV-2 B.1.1.7. (UK, WHO alpha variant), SARS-CoV-2 B.1.351 (South Africa, WHO beta variant), SARS-CoV-2 P.1 (Brazil, WHO gamma variant), SARS-CoV-2 B.1.617.2 (India, WHO delta variant), and SARS-CoV-2 B.1.617.1 (India, WHO kappa variant).

In some embodiments, the method for treating or preventing a disease or disorder in a subject comprises administering to the subject an ACE2-Fc fusion protein that helps to regulate physiological or biological processes normally mediated by endogenous ACE2. In some embodiments, the method for treating or preventing a disease or disorder in a subject comprises administering to the subject an ACE2-Fc fusion protein that reduces the levels of angiotensin I and/or angiotensin II. In some embodiments, the method for treating or preventing a disease or disorder in a subject comprises administering to the subject an ACE2-Fc fusion protein that increases the levels of angiotensin-(1-9) or angiotensin-(1-7). In some embodiments, the method for treating or preventing a disease or disorder in a subject comprises administering to the subject an ACE2-Fc fusion protein that decreases the ratio of angiotensin II to angiotensin-(1-7). In some embodiments, the method for treating or preventing a disease or disorder in a subject comprises administering to the subject an ACE2-Fc fusion protein that decreases the ratio of angiotensin II to angiotensin-(1-7) in about 15 minutes, about 30 minutes, about 1 hour, about 2 hours, about 3 hours, about 4 hours, about 6 hours, about 12 hours, about 18 hours, about 24 hours, about 48 hours, about 72 hours, or about 96 hours. In some embodiments, the method for treating or preventing a disease or disorder in a subject comprises administering to the subject an ACE2-Fc fusion protein that decreases the ratio of angiotensin II to angiotensin-(1-7) by about 5%, about 10%, about 20%, about 25%, about 50%, about 75%, or more.

In some embodiments, the ACE2-Fc fusion protein is administered to treat subjects. As used herein, the term “subject” may be used interchangeably with the term “patient” or “individual” and may include an “animal” and in particular a “mammal”. Subjects that may be treated with the ACE2-Fc fusion protein include, but are not limited to, humans, non-human primates (e.g., monkeys, baboons, and chimpanzees), mice, rats, bovines, horses, household cats, tigers and other large cats, dogs, pigs, rabbits, goats, deer, sheep, ferrets, gerbils, guinea pigs, hamsters, bats, and birds (e.g., chickens, turkeys, and ducks). A number of these household pets and farm animals are capable of carrying and transmitting SARS-CoV-2 or other ACE2-binding viruses without themselves getting substantially sick or dying, thereby transmitting the disease to humans. Thus, in some embodiments, these animals are treated not because they are suffering from disease, but rather, because they can transmit virus to humans and cause human disease. In some embodiments, the human is an adult or a child.

The subject may be a male, or a female. In some embodiments, the subject is greater than about 18 years old, greater than about 25 years old, greater than about 35 years old, greater than about 45 years old, greater than about 55 years old, greater than about 65 years old, greater than about 75 years old, or greater than about 85 years old. In some embodiments, the subject is less than about 18 years old, less than about 16 years old, less than about 14 years old, less than about 12 years old, less than about 10 years old, less than about 8 years old, less than about 6 years old, less than about 5 years old, less than about 4 years old, less than about 3 years old, less than about 2 years old, less than about 1 year old, or less than about 6 months old. In some embodiments, the subject is greater than or equal to 18 years old. In some embodiments, the subject is less than 18 years old.

In some embodiments, the ACE2-Fc fusion proteins of the present disclosure are used to treat a subject with ACE2 deficiency. As used herein, the term “ACE2 deficiency” refers to a subject with insufficient ACE2 enzymatic activity to avoid disease pathology. Such insufficient ACE2 may be absolute, with below average ACE2 baseline expression and/or below average ACE2 enzymatic activity, or may be relative ACE2 deficiency with increased angiotensin II that may be accompanied by normal or nearly normal ACE2 activity levels. In some embodiments, the subject has ACE2 deficiency as a result of genetic, epigenetic, or environmental factors (e.g., SARS-CoV-2). In some embodiments, the subject with ACE2 deficiency is male. In some embodiments, the subject with ACE2 deficiency is female. In some embodiments, the subject with ACE2 deficiency has type A or type AB blood. In some embodiments, the subject with ACE2 deficiency has hypovitaminosis D. In some embodiments, the subject with ACE2 deficiency is Caucasian, African, or Latino (non-Asian). In some embodiments, subjects of Caucasian, African, or Latino (non-Asian) descent have lower levels of ACE2 baseline expression and/or lower ACE2 enzymatic activity compared to subjects of Asian descent. In some embodiments, subjects of Caucasian, African, or Latino (non-Asian) descent have an increased susceptibility to the one or more diseases or disorders described herein compared to subjects of Asian descent. In some embodiments, the subject with ACE2 deficiency is in good physical and mental health (i.e., exhibits no signs or symptoms of a disease or disorder). See, Zhao et al., Am J Respir Crit Care Med, 2020; 202(5):756-759;

In some embodiments, the subject with ACE2 deficiency has increased des-arg⁹-bradykinin, or an elevated ratio of des-arg⁹-bradykinin to des-arg⁹-bradykinin metabolites. Des-arg⁹-bradykinin is processed by ACE2 to des-arg⁹-bradykinin metabolites. Des-arg⁹-bradykinin binds the inflammatory bradykinin B1 receptor (B1R) and des-arg⁹-bradykinin metabolites bind the anti-inflammatory bradykinin B2 receptor (B2R). In some embodiments, the ACE2-Fc fusion protein is used to treat a subject with increased des-arg⁹-bradykinin. In some embodiments, the ACE2-Fc fusion protein is used to treat a subject with increased ratio of des-arg⁹-bradykinin to des-arg⁹-bradykinin metabolites.

In some embodiments, the ACE2-Fc fusion proteins of the present disclosure are administered to a subject with relative ACE2 deficiency. As used herein, the term “relative ACE2 deficiency” refers to a subject with average or above average ACE2 baseline expression and/or average or above average ACE2 enzymatic activity; however, the subject's elevated ACE2 expression level and/or enzymatic activity is insufficient to prevent one or more diseases or disorders in a subject. For example, patients with chronic COVID syndrome have approximately a 100-fold increase in ACE2 activity (See, Patel et al., medRxiv, 2020.10.06.20207514), but the elevated ACE2 activity may not be sufficient if angiotensin II expression is increased 1000-fold. Chronic COVID syndrome is the long-term sequelae of SARS-CoV-2 and subjects suffering from chronic COVID syndrome may have symptoms such as fatigue, headaches, shortness of breath, anosmia, palpitations, chest pain, joint pain, physical limitations, depression, and insomnia. In some embodiments, a subject with relative ACE2 deficiency has increased expression of angiotensin II, increased expression of des-arg⁹-bradykinin, decreased expression of angiotensin-(1-7), and/or decreased expression of des-arg⁹-bradykinin metabolites. In some embodiments, the ACE2-Fc fusion protein of the present disclosure is used to treat a subject with chronic COVID syndrome. In some embodiments, the ACE2-Fc fusion protein of the present disclosure is used to treat a subject with relative ACE2 deficiency.

In some embodiments, the ACE2-Fc fusion proteins of the present disclosure are used to treat or prevent long-haul COVID syndrome. Long-haul COVID is a newly recognized syndrome with evolving definition. Long-haul COVID patients may be deficient in ACE2. Long-haul COVID-19 patients may have persistent symptoms including cognitive issues like ‘brain fog,’ memory or attention problems, shortness of breath, racing heart, nausea, diarrhea, intermittent spiking fevers, and Postural Orthostatic Tachycardia Syndrome (POTS).

In some embodiments, the ACE2-Fc fusion proteins of the present disclosure are used to treat a subject with ACE2 deficiency. In some embodiments, the subject with ACE2 deficiency is middle-aged or advanced-aged. As used herein, “middle-age” refers to subjects between 45 to 59 years old and the term “advanced-age” refers to subjects over 60 years old. In some embodiments, the age of the subject with ACE2 deficiency is between about 45 years old and about 100 years old. In some embodiments, the subject with ACE2 deficiency is more than 45 years old, 50 years old, 55 years old, 60 years old, 65 years old, 70 years old, 75 years old, 80 years old, 85 years old, 90 years old, 95 years old, or 100 years old. In some embodiments, middle-age or advanced-age subjects have an increased susceptibility to the one or more diseases or disorders described herein compared to subjects of a younger age.

In some embodiments, the ACE2-Fc fusion proteins of the present disclosure are administered to a subject with ACE2 deficiency. In some embodiments, the ACE2 deficiency is an absolute deficiency relative to normal values. In some embodiments, the ACE2 deficiency is a relative deficiency wherein the ACE2 measured level may be within normal range but the body is producing insufficient ACE2 as may be indicated, by way of example and not limitation, by increased angiotensin II or increased des-arg⁹-bradykinin. In some embodiments, the subject with ACE2 deficiency is asymptomatically infected with a pathogenic microorganism. In some embodiments, the subject with ACE2 deficiency is asymptomatically infected with a coronavirus, e.g., SARS-CoV-2. In some embodiments, the subject with ACE2 deficiency has been exposed to subject infected with a pathogenic microorganism (e.g., a subject identified through contact tracing).

In some embodiments, the subject is infected or colonized with a microorganism that binds human ACE2. In some embodiments, the subject is infected or colonized with a naturally-evolved microorganism that binds human ACE2. In some embodiments, the naturally-evolved microorganism is a coronavirus (e.g., SARS-CoV-1 or SARS-CoV-2). In some embodiments, the subject is infected or colonized with a human-designed microorganism that binds human ACE2.

In some embodiments, the subject is infected or colonized with a virus. In some embodiments, the subject is infected with a coronavirus such as SARS-CoV-1 or SARS-CoV-2. In some embodiments, the subject may be colonized with more than one strain of SARS-CoV-1 or SARS-CoV-2. In some embodiments, the infection may be an acute infection or a chronic infection. In some embodiments, the subject has elevated levels of angiotensin II and/or increased activation of the inflammatory AT1R pathway due to infection with SARS-CoV-1 or SARS-CoV-2. In some embodiments, the elevated levels of angiotensin II and/or increased activation of the inflammatory AT1R pathway results in one or more chronic diseases or disorders in a subject. See, Miesbach, TH Open, 2020; 4(2):e138-e144; and Zoufaly et al., Lancet Respir Med, 2020, 8(11):1154-1158.

In some embodiments, the subject has (or is suspected of having) one or more diseases or disorders. In some embodiments, the ACE2-Fc fusion protein is used to treat subjects suffering from chronic diseases such as cardiovascular disease, cardiopulmonary disease, lung disease, diabetes-related micro- and macro-vascular diseases, metabolic syndrome, stress-related disorders, endocrine disorders, liver disease, kidney disease, ocular disorders, seizures, multi-organ dysfunction syndrome, inflammation and/or autoimmunity. In some embodiments, the subject has one or more diseases or disorders that can be treated with recombinant human ACE2.

In some embodiments, the ACE2-Fc fusion protein is used to treat or prevent one or more diseases or disorders in a subject with increased activation of the inflammatory AT1R pathway. A subject with “increased activation of the inflammatory AT1R pathway” is a subject with, e.g., increased ACE expression or ACE enzymatic activity, increased expression of angiotensin II, decreased expression of ACE2 or ACE2 enzymatic activity, decreased levels of angiotensin-(1-7), and/or an elevated ratio of angiotensin II to angiotensin-(1-7). Diseases or disorders associated with activation of the inflammatory AT1R pathway include, but are not limited to, acute lung injury (ALI), virus-induced lung injury (e.g., influenza-associated ALI), acute respiratory distress syndrome (ARDS), primary hypertension, pulmonary arterial hypertension, granulomatous diseases (e.g., pulmonary gallium, sarcoidosis, leprosy, histoplasmosis), non-granulomatous diseases (e.g., hyperthyroidism), diabetic and non-diabetic chronic kidney disease, diabetes and diabetic end-organ damage (e.g., diabetic retinopathy, congestive heart failure, diabetes insipidus, and stroke), idiopathic hyperaldosteronism, secondary hyperaldosteronism, primary or secondary hyperparathyroidism, cancer, abdominal aortic aneurysm, obesity, fibrosis (e.g., myocardial fibrosis, renal fibrosis, or pulmonary fibrosis), neurodegenerative disease (e.g., Alzheimer's disease, vascular dementia, Parkinson's disease, and Huntington's disease), arthritis (e.g., rheumatoid arthritis and osteoarthritis), trauma, mood and anxiety disorders, inflammatory bowel disease (e.g., Crohn's disease or ulcerative colitis), intestinal inflammation and diarrhea, endometriosis, syndrome of inappropriate antidiuretic hormone secretion, cardiac hypertrophy, heart failure, vascular hypertrophy, esophageal or diaphragm contractions, sphincter spasms, erectile dysfunction, premature aging, pain, and sepsis.

In some embodiments, the ACE2-Fc fusion proteins of the present disclosure are used to treat or prevent a cardiovascular disease or cardiopulmonary disease in a subject. Examples of cardiovascular or cardiopulmonary diseases include, but are not limited to, hypertension, congestive heart failure, chronic heart failure, acute heart failure, contractile heart failure, myocardial infarction or maladaptive ventricular remodeling after myocardial infarction, cardiac hypertrophy, vascular hypertrophy, chronic bronchitis, atherosclerosis, arteriosclerosis chronic obstructive pulmonary disease (COPD), emphysema, pulmonary arterial hypertension, and pulmonary hypertension. See, Patel et al., Circ Res, 2016; 118(8):1313-26; Kassiri et al., Circ Heart Fail, 2009; 2(5):446-55; Zhong et al., Circulation, 2010; 122(7):717-28; Weber, K T, N Engl J Med, 2001; 6; 345(23):1689-97; Tsuruda et al., Hypertension, 2016; 67(5):848-56; and Griffin et al., Hypertension, 1991; 17:626-635.

In some embodiments, the ACE2-Fc fusion proteins of the present disclosure are used to treat or prevent an abdominal aortic aneurysm. See, Wang et al., J Clin Invest, 2010, 120(2):422-432; and Daugherty and Cassis, Curr Hypertens Rep, 2004; 6(6):442-6.

In some embodiments, the ACE2-Fc fusion proteins of the present disclosure are used to treat or prevent a lung disease in a subject. Exemplary lung diseases include, but are not limited to, Acute Respiratory Distress Syndrome (ARDS), acute lung injury (ALI), virus-induced lung injury (e.g., influenza-associated ALI), toxin-mediated acute lung injury, COPD, pneumonia, asthma, chronic bronchitis, pulmonary emphysema, cystic fibrosis, interstitial lung disease, pulmonary hypertension, pulmonary embolism, pulmonary sarcoidosis, tuberculosis, lung cancer, edema of the lung, and pulmonary hypertonia. See, Huang et al., Nat Comm 2014; 5, Article No. 3595; Zou et al., Nat Comm 2014; 5, Article No. 3594; Imai et al., Nature 2005; 436: 112-116; Li et al., Sci Rep 2016; 15; 6:27911; Weismann et al., J Cereb Blood Flow Metab, 2017; 37(7):2396-2413; Yamazato et al., Hypertension, 2009; 54(2):365-71; and Khan et al., Crit Care, 2017, 21:234

In some embodiments, the ACE2-Fc fusion proteins of the present disclosure are used to treat or prevent a kidney disease in a subject. Examples of kidney diseases include, but are not limited to, renal fibrosis, acute renal failure, chronic renal failure, polycystic kidney disease (PKD), and acute kidney injury. In some embodiments, the ACE2-Fc fusion protein prevents the progression of chronic kidney disease. In some embodiments, the ACE2-Fc fusion protein prevents the progression of acute kidney disease to chronic kidney disease.

In some embodiments, the ACE2-Fc fusion proteins of the present disclosure are used to treat or prevent diabetes in a subject. In some embodiments, the ACE2-Fc fusion protein is used to treat or prevent diabetic end-organ damage in a subject. Examples of diabetic end-organ damage include, but are not limited to, chronic kidney disease, congestive heart failure, myocardial infarction, stroke, hypertension, diabetic retinopathy, peripheral neuropathy, and cutaneous ulceration. In some embodiments, the ACE2-Fc fusion protein is used to treat or prevent diabetes insipidus. In some embodiments, the ACE2-Fc fusion protein is used to treat diabetes in order to prevent chronic kidney disease. See, Bakris et al., New Engl J Med, 2020, 383:2219-2229;

In some embodiments, the ACE2-Fc fusion proteins of the present disclosure are used to treat or prevent obesity in a subject. In some embodiments, the ACE2-Fc fusion proteins of the present disclosure are used to treat or prevent one or more diseases or disorders associated with obesity (e.g., cardiovascular disease and diabetes). See, Patel et al., Diabetes, 2016; 65(1):85-95; Kawabe et al., Am J Physiol Endocrinol Metab, 2019; 317(6):E1140-E1149.

In some embodiments, the ACE2-Fc fusion proteins of the present disclosure are used to treat or prevent a neurodegenerative disease in a subject. Examples of neurodegenerative diseases include, but are not limited to, Alzheimer's disease, vascular dementia, Parkinson's disease, prion disease, motor neuron disease, Huntington's disease, metabolic syndrome, spinocerebellar ataxia, Lewy body disease, Friedreich's ataxia, amytrophic lateral sclerosis, and spinal muscular atrophy. See, Kehoe et al., Alzheimers Res Ther, 2016; 8:50; and Abiodun and Ola, 2020 Saudi J Biol Sci, 27(3):905-912.

In some embodiments, the ACE2-Fc fusion proteins of the present disclosure are used to treat or prevent cognitive impairment associated with a vascular disease in a subject.

In some embodiments, the ACE2-Fc fusion proteins of the present disclosure prevent or treat inflammation in a subject. The inflammation may result from the binding of any pathogenic organism to ACE2, preventing activation of the anti-inflammatory RAAS pathway that balances the inflammatory AT1R pathway. The inflammation may also result from relative ACE2 deficiency accompanying a disease and which may be characterized, by way of example and not limitation, by increased angiotensin II or increased des-arg⁹-bradykinin. The inflammation may be a local inflammation of a tissue or organ and/or a systemic inflammation. The inflammation may be chronic and/or acute inflammation. The inflammation may include, but is not limited to, rheumatism, intestinal inflammation (e.g., inflammatory bowel disease or diarrhea), sepsis, osteoarthritis, rheumatoid arthritis, systemic lupus erythematosus, scleroderma or mixed connective tissue disease. These conditions may be caused by mechanical or chemical cellular or tissue damage, wounds, infections (e.g., viral, bacterial, or fungal infections), implants (e.g., organ or medical implants) and medications. In some embodiments, the inflammation is caused by an infection, such as SARS-CoV-2. See, Hashimoto et al., Nature, 2012, 25; 487(7408):477-81; and Perlot and Penninger, Microbes Infect, 2013; 15(13):866-73.

In some embodiments, the ACE2-Fc fusion protein is used to treat or prevent an inflammatory disease or autoimmune disease in a subject. In some embodiments, the inflammatory disease or autoimmune disease is selected from the group consisting of acquired autoimmune thrombocytopenia, acquired factor VIII autoimmunity, acquired von Willebrand disease, acute idiopathic dysautonomic neuropathy, alloimmune/autoimmune thrombocytopenia, ANCA positive vasculitis, ankylosing spondylitis, anti-decorin (BJ antigen) myopathy, aplastic anemia, asthma, atopic dermatitis, autoimmune anemia, autoimmune hemolytic anemia, autoimmune neutropenia, autoimmune thyroiditis, autoimmune uveitis, bone marrow transplantation rejection, celiac disease, chronic inflammatory demyelinating polyneuropathy (CIDP), chronic inflammatory demyelinating polyradiculoneuropathy, chronic lymphocytic leukemia (CLL), Crohn's disease, Cushing syndrome, dermatomyositis, dermatopolymyositis, diabetic neuropathy, Diamond-Blackfan anemia, epilepsy, Evan's syndrome, Felty's syndrome, Gaucher's disease, Goodpasture's disease, Grave's disease, Guillain-Barré syndrome, hemolytic disease of the newborn, hemolytic uremic syndrome, idiopathic thrombocytopenic purpura (ITP), immune-mediated neutropenia, inclusion-body myositis, inflammatory bowel disease, inflammatory myopathies, juvenile idiopathic arthritis, Kawasaki disease, Lambert-Eaton myasthenic syndrome, lower motor neuron syndrome associated with anti-GM1, monoclonal gammopathy of unknown significance, multifocal motor neuropathy (MMN), multiple sclerosis, myasthenia gravis, myelitis, myositis, necrotizing fasciitis, optic neuritis, organ transplantation rejection, Paget's disease, paraneoplastic cerebellar degeneration with anti-Yo antibodies, paraneoplastic encephalomyelitis, paraneoplastic necrotic myopathy, paraproteinemic IgM demyelinating polyneuropathy, pemphigus, penacillamine induced polymyositis, post-transfusion purpura, psoriasis, pure red cell aplasia, reactive arthritis, refractoriness to platelet transfusion, rheumatoid arthritis, sarcoidosis, scleroderma, sclerosing cholangitis, sensory neuropathy with anti-Hu antibodies, sepsis, sickle cell crisis, spondyloarthropathies, spontaneous polymyositis, Stiff Man Syndrome, systemic lupus erythematosus (SLE), systemic vasculitis, thrombotic thrombocytopenia purpura, type I diabetes mellitus, ulcerative colitis, Wegener's granulomatosis, Whipple's disease, and X-linked vacuolated myopathy. See, Jacobs et al., Dig Dis Sci, 2019; 64(7):1938-1944.

In some embodiments, the ACE2-Fc fusion proteins of the present disclosure are used to treat or prevent arthritis in a subject. In some embodiments, the ACE2-Fc fusion protein is used to treat or prevent rheumatoid arthritis. In some embodiments, the ACE2-Fc fusion protein is used to treat or prevent osteoarthritis. Additional examples of arthritic diseases include, but are not limited to, juvenile rheumatoid arthritis, gout, patellofemoral arthritis, chondromalacia, axial and peripheral spondyloarthropathies (e.g., ankylosing spondylitis and enteropathic arthritis), systemic lupus erythematosus, and psoriatic arthritis. See, Kawakami et al., Arthritis, 2012, Article No. 648537; and Walsh et al., Ann Rheum Dis, 2000; 59(2):125-131;

In some embodiments, the ACE2-Fc fusion proteins of the present disclosure are used to treat or prevent one or more endocrine disorders in a subject. Examples of endocrine disorders include, but are not limited to, hyperaldosteronism, type 1 or type 2 diabetes, osteoporosis, thyroid cancer, Addison's disease, Cushing's syndrome, Grave's disease, and Hashimoto's thyroiditis. In some embodiments, the ACE2-Fc fusion protein is used to treat or prevent hyperaldosteronism. In some embodiments, the ACE2-Fc fusion protein is used to treat or prevent secondary hyperaldosteronism caused by a renin-producing tumor, renal artery stenosis, or edematous disorders (e.g., left ventricular heart failure, pregnancy, cor pulmonale, or cirrhosis with ascites). In some embodiments, the ACE2-Fc fusion protein is used to treat or prevent primary hyperparathyroidism. In some embodiments, the ACE2-Fc fusion protein is used to treat or prevent secondary hyperparathyroidism. See, Wisgerhof et al., J Clin Endo Metab, 1978, 47(5):938-943; and Lenzini et al., J Clin Endocrinol Metab, 2019; 104(9):3726-3734.

In some embodiments, the ACE2-Fc fusion proteins of the present disclosure are used to treat or prevent fibrosis. In some embodiments, the fibrosis is a local fibrosis of a tissue or organ-specific fibrosis. Such organ-specific fibroses include, but are not limited to, hepatic fibroses, pulmonary fibroses, connective tissue fibroses (e.g., muscle septa) renal fibrosis, and fibrosis of the skin. In some embodiments, the fibrosis is a fibrosis of an internal organ, e.g., the liver, kidneys, lungs, heart, stomach, intestines, pancreas, glands, muscles, cartilage, tendons, ligaments or joints. In some embodiments, the fibrosis is cystic fibrosis or rheumatic fibrosis. In some embodiments, the fibrosis occurs concurrently with inflammation, e.g., hepatitis (inflammatory liver disease). In some embodiments, the fibrosis is associated with organ transplantation. In some embodiments, the ACE2-Fc fusion protein is used to treat or prevent myocardial fibrosis, renal fibrosis, or pulmonary fibrosis in a subject. In some embodiments, the pulmonary fibrosis is chronic obstructive pulmonary disease (COPD) or idiopathic pulmonary fibrosis. In some embodiments, the fibrosis is the result of chemotherapy (e.g., bleomycin, carboplatin, or vinorelbine), occupational exposure (e.g., asbestos or silica), autoimmune disease (e.g., rheumatoid arthritis, scleroderma, or Sjogren's syndrome), viral infection (e.g., SARS-CoV-2), or gastroesophageal reflux disease. See, Sopel et al., Laboratory Investigation, 2011, 91:565-578; Lavoz et al., PLoS One, 2012, 7(7):e40490; and Uhal et al., Am J Physiol Lung Cell Mol Physiol, 2011; 301(3):L269-74.

In some embodiments, the ACE2-Fc fusion proteins of the present disclosure are used to treat or prevent chronic fibrosis. The fibrosis may be caused by mechanical or chemical cell or tissue damage, wounds, cancer, infections (e.g., viruses, bacteria or fungi), implants (e.g., organ implants or medical devices), and medications. Infections that cause chronic fibrosis may be organ-specific, such as hepatitis virus infection (e.g., HCV). Other chronic fibrotic diseases, which may be treated with the ACE2-Fc fusion proteins of the present disclosure, include, for example, primary or secondary fibroses, in particular fibroses caused by an autoimmune response and Ormond's disease (retroperitoneal fibrosis). In some embodiments, the fibrosis occurs concurrently with inflammation. See, Weng et al., Cell Physiol Biochem. 2015; 36(2):697-711.

In some embodiments, the ACE2-Fc fusion proteins of the present disclosure are used to treat or prevent a liver disease. Examples of liver disease include, but are not limited to, hepatitis, fatty liver disease, cirrhosis, liver cancer, hemochromatosis, and Wilson disease. In some embodiments, the liver disease occurs concurrently with inflammation, e.g., hepatitis (inflammatory liver disease). See, Warner F J et al., Clin Sci (Lond)2020 Dec. 11; 134(23):3137-3158.

In some embodiments, the ACE2-Fc fusion proteins of the present disclosure are used to treat or prevent apoptotic disease. Examples of apoptotic diseases include but are not limited to, cancers, mitochondrial diseases, Parkinson's disease, or Alzheimer's disease. See, Bao H et al., Cell Physiol Biochem 2015; 37:759-767

In some embodiments, the ACE2-Fc fusion proteins of the present disclosure are used to treat or prevent tumor diseases and cancer. In some embodiments, the ACE2-Fc fusion protein is used to treat or prevent metastatic cancer. Examples of tumor diseases and cancer include, but are not limited to, tumor diseases of the reproductive tract, in particular ovarian cancer, testicular cancer, prostate cancer or breast cancer, tumor diseases of the digestive tract, in particular stomach cancer, intestinal cancer, rectum carcinoma, pancreatic cancer, esophagus cancer, and liver cancer, kidney cancer, lung cancer, melanomas, endometrial cancer, or neuroblastomas. See, Ishikane and Takahashi-Yanaga, Biochem Pharmcol, 2019; 151:96-103; and Anandanadesan et al., J Gastrointest Surg, 2008; 12(1):57-66.

In some embodiments, the ACE2-Fc fusion proteins of the present disclosure are used to treat or prevent a granulomatous disease. Examples of granulomatous diseases include, but are not limited to, sarcoidosis, pulmonary gallium, leprosy, and histoplasmosis. In some embodiments, the ACE2-Fc fusion protein is used to treat or prevent a non-granulomatous disease, e.g., hyperthyroidism. See, Cohen et al., Thorax, 1985; 40(7):497-500; Janssen et al., Chest, 2003; 124(6):2119-25; Yotsumoto et al., Ann Intern Med, 1982; 96(3):326-8.

In some embodiments, the ACE2-Fc fusion proteins of the present disclosure are used to treat or prevent endometriosis in a subject. In some embodiments, the ACE2-Fc fusion protein is used to treat pain resulting from endometriosis in a subject. See, Abraham et al., PLoS One, 2012; 7(5):e37750.

In some embodiments, the ACE2-Fc fusion proteins of the present disclosure are used to treat or prevent pain in a subject. In some embodiments, the ACE2-Fc fusion protein is used to treat or prevent nociceptive pain in a subject. In some embodiments, the ACE2-Fc fusion protein is used to treat or prevent inflammatory pain in a subject. In some embodiments, the ACE2-Fc fusion protein is used to treat or prevent neuropathic pain in a subject. In some embodiments, the ACE2-Fc fusion protein is used to treat or prevent functional pain in a subject. See, Shiers S. Pain 2020 November; 161(11): 2494-2501.

In some embodiments, the ACE2-Fc fusion proteins of the present disclosure are used to treat or prevent erectile dysfunction in a subject. See, Zhang et al., Cell Physiol Biochem,

In some embodiments, the ACE2-Fc fusion proteins of the present disclosure are used to treat or prevent premature aging in a subject. In some embodiments, the ACE2-Fc fusion protein is used to treat Hutchinson-Gilford syndrome or Werner syndrome. In some embodiments, the ACE2-Fc fusion protein is used to treat cardiovascular aging. See, Cooper et al., Circ Res, 2018; 123 (6): 651-653.

In some embodiments, the ACE2-Fc fusion proteins of the present disclosure are used to treat or prevent disorders characterized by excess retention of sodium. In some embodiments, the excess retention of sodium is associated with diseases or disorders such as hypertension, pulmonary artery hypertension, peripheral edema, hyperventilation, or the use of steroids, licorice, or blood pressure lowering medicines. See, Fountain and Lappin, StatPearls [Internet], 2020.

In some embodiments, the ACE2-Fc fusion proteins of the present disclosure are used to treat or prevent dehydration. Dehydration may result from excess release of vasopressin and/or increased angiotensin II. In some embodiments, the ACE2-Fc fusion protein is used to treat or prevent excess release of vasopressin. In some embodiments, the ACE2-Fc fusion protein is used to regulate cardiovascular and blood pressure regulation. In some embodiments, the ACE2-Fc fusion protein is used to treat or prevent the syndrome of inappropriate antidiuretic hormone (SIADH). In some embodiments, the ACE2-Fc fusion protein is used to treat or prevent diabetes insipidus. See, Carmona-Calero et al., Advances in Endocrinology, 2014; Article No. 179795; and Fountain and Lappin, StatPearls [Internet], 2020.

In some embodiments, the ACE2-Fc fusion proteins of the present disclosure are used to treat or prevent esophageal contractions in a subject. In some embodiments, the esophageal contractions are the result of gastroesophageal reflux disease, reflux esophagitis, diffuse esophageal spasm and other disorders of esophageal motility, and achalasia. In some embodiments, the ACE2-Fc fusion is used to treat or prevent diaphragm contractions (e.g., hiccups) in a subject. See, Casselbrant et al., Gastroenterology, 2007; 132:249:260.

In some embodiments, the ACE2-Fc fusion proteins of the present disclosure are used to treat or prevent sphincter spasms in a subject. In some embodiments, the ACE2-Fc fusion protein is used to treat or prevent an anal sphincter spasm, a pelvic sphincter spasm, an ureteral sphincter spasm, or a bladder sphincter spasm. In some embodiments, the ACE2-Fc fusion protein is used to treat or prevent constipation, bladder outlet obstruction, or urinary outflow obstruction caused by a sphincter spasm. See, Yamada et al., Eur Urol, 2009, 55(2):482-9.

In some embodiments, the ACE2-Fc fusion proteins of the present disclosure are used to treat a subject that has experienced a trauma. In some embodiments, the trauma is an acute trauma, a complex trauma (a subject that has experienced multiple traumatic events with wide-ranging and long-term effects), or a chronic trauma. In some embodiments, the ACE2-Fc fusion protein is used to treat a subject with post-traumatic stress disorder. In some embodiments, the ACE2-Fc fusion protein is used to treat a subject that has trauma from child abuse, bullying, or domestic violence. See, Brudey et al., Am J Physiol Regul Integr Comp Physiol, 2015; 309(4):R315-R321; and Seligowski et al., Neuropsychopharmacol (2020).

In some embodiments, the ACE2-Fc fusion proteins of the present disclosure are used to treat a subject with a mood or anxiety disorder. Examples of mood and anxiety disorders include, but are not limited to, major depressive disorder, post-partum depression, seasonal affective disorder, premenstrual dysphoric disorder, bipolar disorder, generalized anxiety disorder, obsessive compulsive disorder, panic disorder, and social anxiety disorder. See, Liu et al., Int J Physiol Pathophysiol Pharmacol, 2012; 4(1):28-35.

In some embodiments, the ACE2-Fc fusion proteins of the present disclosure are used to treat or prevent chronic fatigue syndrome. In some embodiments, the ACE2-Fc fusion protein is used to treat or prevent dysautonomia. In some embodiments, the ACE2-Fc fusion protein is used to treat or prevent neurocardiogenic syncope. In some embodiments, the ACE2-Fc fusion protein is used to treat or prevent postural orthostatic tachycardia syndrome (POTS). See, Stewart J M et al., Hypertension. 2009 May; 53(5): 767-774.

In some embodiments, the ACE2-Fc fusion proteins of the present disclosure are used to treat a disease or disorder treated with steroid therapies. In some embodiments, the ACE2-Fc fusion protein is used to treat a disease or disorder treated with prednisolone therapy. See, Baughman et al., Am Rev Respir Dis, 1983; 128(4):631-3.

In some embodiments, the present disclosure provides a method of treating a disease or disorder in a subject in need thereof comprising detecting a level of angiotensin II (Ang II), detecting a ratio of Ang II to Ang 1-7, or detecting a level of des-arg-9-bradykinin in the subject and administering the ACE2-Fc fusion protein to the subject if an elevated level of Ang II or des-arg-9-bradykinin, or an elevated Ang II/Ang 1-7 ratio is detected.

In some embodiments, the present disclosure provides a method of treating a disease or disorder in a subject in need thereof comprising detecting a level of angiotensin II (Ang II), in the subject and administering the ACE2-Fc fusion protein to the subject if an elevated level of Ang II is detected.

In some embodiments, the present disclosure provides a method of treating a disease or disorder in a subject in need thereof comprising detecting a level of des-arg-9-bradykinin in the subject and administering the ACE2-Fc fusion protein to the subject if an elevated level of des-arg-9-bradykinin is detected.

In some embodiments, the present disclosure provides a method of treating a disease or disorder in a subject in need thereof comprising detecting a ratio of Ang II to Ang 1-7 administering the ACE2-Fc fusion protein to the subject if an elevated level Ang II/Ang 1-7 ratio is detected.

In some embodiments, an elevated level of Ang II, Ang 1-7, or des-arg-9-bradykinin is determined based on a comparison to the subject's historical levels of one of these factors (i.e., a level of the factor detected in the subject prior to diagnosis with the disease or disorder to be treated with the ACE2-Fc fusion protein). In some embodiments, an elevated, normal, or decreased level of Ang II, Ang 1-7, and/or des-arg-bradykinin is determined based on comparison to the level observed in a healthy control population.

The normal range for Ang II levels in plasma is less than 5-40 pg/L using current test methods. See the Test Catalog available at the Mayo Clinic Laboratories website. In some embodiments, an elevated level of Ang II is greater than about 35 pg/L, about 40 pg/L, about 45 pg/L, about 50 pg/L, about 60 pg/L, about 70 pg/L, about 80 pg/L, about 90 pg/L, or about 100 pg/L. In some embodiments, an elevated level of Ang II is an increase in Ang II levels of about 20%, about 30%, about 40%, about 50%, about 60%, about 70%, about 80%, about 90%, about 100% or more from a subject's historical Ang II level (i.e., a level of Ang II determined prior to diagnosis with a disease to be treated by the ACE2-Fc fusion proteins described herein) or from the normal level observed in a healthy control population.

The normal range for Ang 1-7 in plasma is less than 10-55 pg/mL using current test methods. See the Test Catalog available at the Mayo Clinic Laboratories website. In some embodiments, a decreased level of Ang 1-7 is less than about 15 pg/L, about 10 pg/L, about 5 pg/L, or about 1 pg/L. In some embodiments, a decreased level of Ang 1-7 is a decrease in Ang 1-7 levels of about 20%, about 30%, about 40%, about 50%, about 60%, about 70%, about 80%, about 90%, about 100% or more from a subject's historical Ang 1-7 level (i.e., a level of Ang 1-7 determined prior to diagnosis with a disease to be treated by the ACE2-Fc fusion proteins described herein) or from the normal level observed in a healthy control population.

The normal range for des-arg-9-bradykinin is about 80-176 pmol/mL using current test methods (See Fernandes et al., Hypertens Res. 2021 Feb. 10. doi: 10.1038/s41440-021-00618-0. Epub ahead of print. PMID: 33568792). In some embodiments, an elevated level of des-arg-9-bradykinin is greater than about 165 pmol/mL, 175 pmol/mL, 185 pmol/mL, 195 pmol/mL, 205 pmol/mL, 215 pmol/mL, 225 pmol/mL, 235 pmol/mL, 245 pmol/mL, 255 pmol/mL, 265 pmol/mL, 275 pmol/mL, 285 pmol/mL, 295 pmol/mL, or greater than about 305 pmol/mL. In some embodiments, an elevated level of des-arg-9-bradykinin is a change of about 20%, about 30%, about 40%, about 50%, about 60%, about 70%, about 80%, about 90%, about 100% or more from a subject's historical des-arg-9-bradykinin level (i.e., a level of des-arg-9-bradykinin determined prior to diagnosis with a disease to be treated by the ACE2-Fc fusion proteins described herein) or from the normal level observed in a healthy control population.

In some embodiments, the subject is treated with an ACE2-Fc fusion protein described herein if the subject demonstrates an increase in the ratio of Ang II to Ang 1-7. This can occur via an increase in Ang II levels with no change in Ang 1-7 levels, or a decrease in Ang 1-7 levels with no change in Ang II levels.

In some embodiments, the ACE2-Fc fusion protein is administered to the subject in one or more doses until the elevated levels of Ang II and/or des-arg-9-bradykinin are reduced by at least 20%, at least 30%, at least 40%, at least 50%, at least 60%, at least 70%, at least 80%, at least 90%, or at least 100%. In some embodiments, the ACE2-Fc fusion protein is administered to the subject in one or more doses until the elevated levels of Ang II and/or des-arg-9-bradykinin are reduced to the normal range for either factor. In some embodiments, the the ACE2-Fc fusion protein is administered to the subject in one or more doses until the Ang II/Ang 1-7 ratio is reduced to within 20% variance from the normal range, is reduced to within 10% variance from the normal range, or is reduced to the normal range.

The pharmaceutical compositions described herein may be administered at a therapeutically-effective dose. As used herein, “therapeutically-effective dose” means a dose sufficient to achieve the intended therapeutic purpose, such as, to alleviate a sign or symptom of a disease or disorder in a subject. A therapeutically effective amount of ACE2-Fc fusion protein will vary with the particular goal to be achieved, the age and physical condition of the subject being treated, the severity of the underlying disease, the duration of treatment, the nature of concurrent therapy and the specific compound employed. For example, a therapeutically effective amount of ACE2-Fc fusion protein administered to a child or a neonate will be reduced proportionately in accordance with sound medical judgement. The effective amount of ACE2-Fc fusion protein will thus be the minimum amount which will provide the desired effect.

The amount of ACE2-Fc fusion protein administered will depend upon a variety of factors, including, for example, the particular indication being treated, the route of administration, whether the desired benefit is prophylactic or therapeutic, the severity of the indication being treated and the age and weight of the patient, the bioavailability of the particular active compound, and the like. Determination of an effective dosage is well within the capabilities of those skilled in the art.

Dosage amounts of the ACE2-Fc fusion proteins disclosed herein will typically be in the range of from about 0.0001 mg/kg/day to about 1000 mg/kg/day, but can be higher or lower, depending upon, among other factors, the activity of the compound, its bioavailability, the mode of administration, and various factors discussed above. In some embodiments, the dose is from about 0.0001 mg/kg to about 1000 mg/kg of body weight per day. In some embodiments, the dose is from about 0.001 mg/kg to about 1000 mg/kg of body weight per day. In some embodiments, the dose is from about 0.01 mg/kg to about 1000 mg/kg of body weight per day. In some embodiments, the dose is from about 0.1 mg/kg to about 100 mg/kg of body weight per day. In some embodiments, the dose is from about 0.5 mg/kg to about 50 mg/kg of body weight per day. In some embodiments, the dose is from about 1 mg/kg to about 25 mg/kg of body weight per day. In some embodiments, the dose is from about 5 mg/kg to about 15 mg/kg of body weight per day. In some embodiments, the dose is about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. Dosage amount and interval can be adjusted individually to provide plasma levels of the compound(s) which are sufficient to maintain therapeutic or prophylactic effect. In cases of local administration or selective uptake, such as local topical administration, the effective local concentration of active compound(s) cannot be related to plasma concentration. Skilled artisans will be able to optimize effective local dosages without undue experimentation.

In some embodiments, the dose of ACE2-Fc fusion protein required to increase ACE2 enzymatic activity and/or decrease angiotensin II expression in a subject is less than the dose of ACE2-Fc fusion protein required to bind and neutralize a pathogenic microorganism. In some embodiments, the dose of ACE2-Fc fusion protein required to decrease angiotensin II expression in a subject infected with a coronavirus is less than the dose of ACE2-Fc fusion protein required to bind and neutralize the coronavirus. In some embodiments the dose of ACE2-Fc fusion protein required to decrease angiotensin II expression in a subject is about 25 mg/kg, about 20 mg/kg, about 15 mg/kg, about 10 mg/kg, about 5 mg/kg, about 1 mg/kg, or less. In some embodiments the dose of ACE2-Fc fusion protein required to neutralize the coronavirus in a subject is about 10 mg/kg, about 15 mg/kg, about 20 mg/kg, about 25 mg/kg, about 50 mg/kg, about 75 mg/kg, about 100 mg/kg, or more. In some embodiments, the dose of ACE2-Fc fusion protein required to bind and neutralize a coronavirus in a subject is greater than the dose of ACE2-Fc fusion protein required decrease angiotensin II expression in a subject infected with the coronavirus. In some embodiments, the dose of ACE2-Fc fusion protein required to bind and neutralize the coronavirus in a subject is 2-fold, 5-fold, 10-fold, 25-fold, 50-fold, 100-fold, or greater than the dose of ACE2-Fc fusion protein required decrease angiotensin II expression in a subject. In some embodiments, the coronavirus is SARS-CoV-1 or SARS-CoV-2.

The ACE2-Fc fusion proteins can be administered once per day, once per week, or multiple times per day or week. Administration frequency may depend upon, among other things, the indication being treated and the judgment of the prescribing physician. A treatment of a subject with a therapeutically effective amount of a compound can include a single treatment or, preferably, can include a series of treatments. In another example, a subject may be treated daily for several years in the setting of a chronic condition or illness. It will also be appreciated that the effective dosage used for treatment may increase or decrease over the course of a particular treatment.

In some embodiments, a tissue or blood sample from a subject is assessed for one or more components of the renin-angiotensin-aldosterone system (RAAS). In some embodiments, a tissue or blood sample from a subject is assessed for angiotensinogen, angiotensin I, angiotensin II, angiotensin-(1-7), angiotensin-(1-9), angiotensin-(1-5), angiotensin(1-8), the AT1 receptor, the AT2 receptor, the MAS receptor, ACE, ACE2, des-arg⁹-bradykinin, des-arg⁹-bradykinin metabolites, and/or renin. In some embodiments, a tissue or blood sample from a subject is assessed for one or more components of the RAAS system and the results are used to determine whether or not to treat the subject with an ACE2-Fc fusion protein. In some embodiments, a tissue or blood sample from a subject is assessed for one or more components of the RAAS system and the results are used to determine the dosing regimen of a subject being treated with an ACE2-Fc fusion protein. In some embodiments, a tissue or blood sample from a subject is assessed for ACE2 expression and/or ACE2 enzymatic activity and the results are used to determine whether or not to treat the subject with an ACE2-Fc fusion protein. In some embodiments, a tissue or blood sample from a subject is assessed for ACE2 expression and/or ACE2 enzymatic activity and the results are used to determine the dosing regimen of a subject being treated with an ACE2-Fc fusion protein.

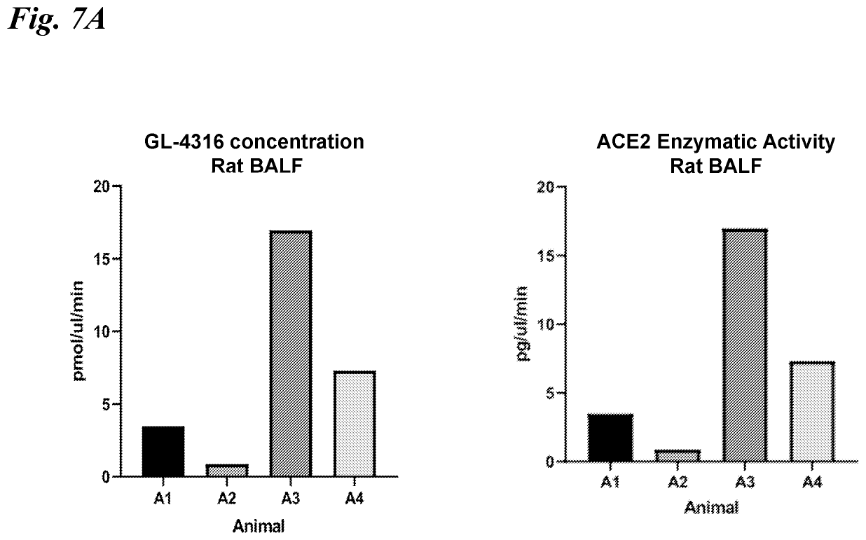

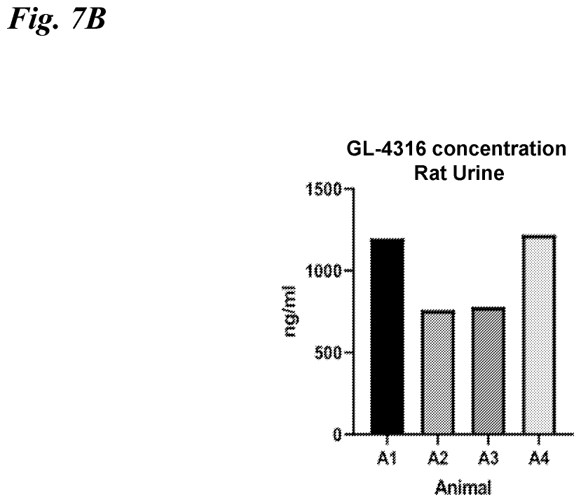

Some embodiments of the disclosure are optimized for transit to peripheral tissue and in some embodiments, the ACE2-Fc fusion protein is detected in peripheral tissue (e.g., lung, kidney, and heart) following administration to a subject. Optimization of peripheral delivery of compounds of this disclosure is achieved in part through the use of the Fc which enables neonatal receptor-mediated transcytosis. In some embodiments, the ACE2 portion of the Fc fusion of the current invention is a truncated ACE2 extracellular domain. In some embodiments, the truncated ACE2 extracellular domain retains most or all of the ACE2 enzymatic activity but because of its smaller size demonstrates increased transit to peripheral tissues. In some embodiments, the enzymatically active truncated ACE2 extracellular domain that transits to peripheral tissue has the SEQ ID No: 8. In some embodiments, the ACE2-Fc fusion protein is detected in the urine following administration to a subject. In some embodiments, the ACE2-Fc fusion protein is detected in the serum following administration to a subject. In some embodiments, the ACE2-Fc fusion protein is detected in the bronchoalveolar lavage fluid (BALF) following administration to a subject. In some embodiments, the ACE2-Fc fusion protein is isolated from the serum, urine, sputum, feces, ocular fluid, and/or BALF of a subject. In some embodiments, the isolated ACE2-Fc fusion protein isolated from the serum, urine, and/or BALF of a subject is subjected to an ACE2 enzymatic activity assay (See, e.g., FIG. 7B). In some embodiments, the ACE2-Fc fusion protein isolated from the serum, urine, and/or BALF of a subject is enzymatically active (See, e.g., FIG. 7A).

In some embodiments, the ACE2-Fc fusion protein has increased delivery to peripheral tissue (e.g., lung, kidney, and heart) compared to recombinant human ACE2 following administration to a subject. In some embodiments, the ACE2 extracellular domain or fragment thereof of the ACE2-Fc fusion protein increases delivery to peripheral tissue compared to recombinant human ACE2. In some embodiments, the IgG4 Fc domain or mutated IgG1 or IgG3 Fc domain of the ACE2-Fc fusion protein increases delivery to peripheral tissue compared to recombinant human ACE2. In some embodiments, the ACE2 extracellular domain or fragment thereof and the IgG4 Fc domain or mutated IgG1 or IgG3 Fc domain of the ACE2-Fc fusion protein increases delivery to peripheral tissue in a treated subject compared to recombinant human ACE2. In some embodiments, the ACE2-Fc fusion protein binds to the neonatal receptor FcRn. In some embodiments, the ACE2-Fc fusion protein binds to FcRn and is transcytosed by the host cell, resulting in increased delivery to peripheral tissue.

The ACE2-Fc fusion proteins of the present disclosure can be administered before, during or after administration of one or more additional therapeutic agents. In some embodiments, the one or more additional therapeutic agents are selected from the group consisting of direct-acting antiviral agents; immune modulators; a steroid, a biologic such as a monoclonal antibody, a fusion protein, or an anti-cytokine; a non-biologic; an immunosuppressant; an antibiotic; a cytokine; or an agent otherwise capable of acting as an immune-modulator. Examples of direct-acting antiviral agents include, but are not limited to, remdesivir, EIDD-2801, nucleoside/tide analogues, nucleocapsid inhibitors, anti-spike protein monoclonal or polyclonal antibodies, convalescent plasma, and interferon. Examples of immune modulators include, but are not limited to, JAK inhibitors, pooled human IVIG, recombinant mimetics of IVIG, a multimerized or aggregated therapeutic comprising Fc domains, monoclonal or bispecific antibodies against one or more TNF superfamily member of cytokines, IL-6, or IL-1, and BTK inhibitors. Examples of steroids include, but are not limited to, prednisone, prednisolone, cortisone, dexamethasone, mometasone testosterone, estrogen, oxandrolone, fluticasone, budesonide, beclamethasone, albuterol, or levalbuterol. In some embodiments, the monoclonal antibody is eculizumab, infliximab, adalimumab, rituximab, tocilizumab, golimumab, ofatumumab, LY2127399, belimumab, veltuzumab, mepolizumab, necitumumab, nivolumab, dinutuximab, secukinumab, evolocumab, blinatumomab, pembrolizumab, ramucirumab, vedolizumab, siltuximab, obinutuzumab, adotrastuzumab, raxibacumab, pertuzumab, brentuximab, ipilumumab, denosumab, canakinumab, ustekinumab, catumaxomab, ranibizumab, panitumumab, natalizumab, bevacizumab, cetuximab, efalizumab, omalizumab, toitumomab-I131, alemtuzumab, gemtuzumab, trastuzumab, palivizumab, basilixumab, daclizumab, abciximab, murononomab or certolizumab. In some embodiments, the fusion protein is etanercept or abatacept. In some embodiments, the anti-cytokine biologic is anakinra. In some embodiments, the non-biologic drug is cyclophosphamide, methotrexate, azathioprine, hydroxychloroquine, leflunomide, minocycline, organic gold compounds, fostamatinib, tofacitinib, etoricoxib, or sulfasalazine. In some embodiments, the immunosuppressant is cyclosporine A, tacrolimus, sirolimus, mycophenolate mofetil, everolimus, OKT3, antithymocyte globulin, basiliximab, daclizumumab, or alemtuzumab. Other examples of additional therapeutic agents that can be administered to a subject in combination with the ACE2-Fc fusion proteins disclosed herein include a non-steroidal anti-inflammatory agent (NSAID) or related inhibitor of cyclooxygenase, aspirin or a related inhibitor of prostaglandin, cannabidiol, salsalate, colchicine, quinine, allopurinol, and statins.

In some embodiments, the ACE2-Fc fusion protein is administered before, during or after administration of the additional therapeutic agent. In some embodiments, the ACE2-Fc fusion protein is administered prior to the administration of the additional therapeutic against. In some embodiments, the ACE2-Fc fusion protein is administered at the same time as the administration of the additional therapeutic agent. In some embodiments, the ACE2-Fc fusion protein is administered after the administration of the additional therapeutic agent. In some embodiments, the ACE2-Fc fusion protein and the additional therapeutic agent display therapeutic synergy when administered in combination.

### Protein Expression Systems

In some embodiments, a vector comprising an expression cassette comprising a polynucleotide sequence encoding the ACE2-Fc fusion protein described herein is introduced into a host cell that is capable of expressing the encoded ACE2-Fc fusion protein. Exemplary host cells include Chinese Hamster Ovary (CHO) cells, HEK 293 cells, BHK cells, murine NSO cells, or murine SP2/0 cells, and E. coli cells. The expressed protein is then purified from the culture system using any one of a variety of methods known in the art (e.g., Protein A columns, affinity chromatography, size-exclusion chromatography, ion exchange chromatography, hydrophobic interaction chromatography and the like).

Numerous expression systems exist that are suitable for use in producing the ACE2-Fc fusion proteins described herein. Eukaryote-based systems in particular can be employed to produce polypeptides, proteins and peptides. Many such systems are commercially and widely available.

In some embodiments, the ACE2-Fc fusion proteins described herein are produced using Chinese Hamster Ovary (CHO) cells following standardized protocols.

The insect cell/baculovirus system can produce a high level of protein expression of a heterologous nucleic acid segment, such as described in U.S. Pat. Nos. 5,871,986 and 4,879,236, both incorporated herein by reference in their entireties, and which can be bought, for example, under the name MAXBAC® 2.0 from Invitrogen and BACPACK™ Baculovirus expression system from Takara Bio.

Other examples of expression systems include Stratagene's Complete Control Inducible Mammalian Expression System, which utilizes a synthetic ecdysone-inducible receptor. Another example of an inducible expression system is available from Invitrogen, which carries the T-REX™ (tetracyclineregulated expression) System, an inducible mammalian expression system that uses the full-length CMV promoter. Invitrogen also provides a yeast expression system called the Pichia methanolica Expression System, which is designed for high-level production of recombinant proteins in the methylotrophic yeast Pichia methanolica. One of skill in the art would know how to express vectors such as an expression construct comprising a nucleic acid sequence encoding a ACE2-Fc fusion protein described herein, to produce its encoded nucleic acid sequence or its cognate polypeptide, protein, or peptide. See, generally, Recombinant Gene Expression Protocols By Rocky S. Tuan, Humana Press (1997), ISBN 0896033333; Advanced Technologies for Biopharmaceutical Processing By Roshni L. Dutton, Jeno M. Scharer, Blackwell Publishing (2007), ISBN 0813 805171; Recombinant Protein Production With Prokaryotic and Eukaryotic Cells By Otto-Wilhelm Merten, Contributor European Federation of Biotechnology, Section on Microbial Physiology Staff, Springer (2001), ISBN 0792371372.

As an alternative, ACE2-Fc fusion proteins of the present invention can be synthesized by exclusive solid phase synthesis, partial solid phase methods, fragment condensation or classical solution synthesis. These synthesis methods are well-known to those of skill in the art (See, for example, Merrifield, J. Am. Chem. Soc. 85:2149 (1963), Stewart et al., “Solid Phase Peptide Synthesis” (2nd Edition), (Pierce Chemical Co. 1984), Bayer and Rapp, Chem. Pept. Prot. 3:3 (1986), Atherton et al., Solid Phase Peptide Synthesis: A Practical Approach (IRL Press 1989), Fields and Colowick, “Solid-Phase Peptide Synthesis,” Methods in Enzymology Volume 289 (Academic Press 1997), and Lloyd-Williams et al., Chemical Approaches to the Synthesis of Peptides and Proteins (CRC Press, Inc. 1997)). Variations in total chemical synthesis strategies, such as “native chemical ligation” and “expressed protein ligation” are also standard (see, for example, Dawson et al., Science 266:776 (1994), Hackeng et al., Proc. Nat'l Acad. Sci. USA 94:7845 (1997), Dawson, Methods Enzymol. 287: 34 (1997), Muir et al, Proc. Nat'l Acad. Sci. USA 95:6705 (1998), and Severinov and Muir, J. Biol. Chem. 273:16205 (1998)). In one example of expressed protein ligation, a recombinantly expressed protein is cleaved from an intein and the protein is ligated to a peptide containing an N-terminal cysteine having an unoxidized sulfhydryl side chain, by contacting the protein with the peptide in a reaction solution containing a conjugated thiophenol. This forms a C-terminal thioester of the recombinant protein which spontaneously rearranges intramolecularly to form an amide bond linking the protein to the peptide. See, generally, Muir, T W et al. Expressed Protein Ligation: A General Method for Protein Engineering, PNAS (1998) 95(12)6705-6710; U.S. Pat. No. 6,849,428; US Pub. 2002/0151006; Bondalapati, et al., Expanding the chemical toolbox for the synthesis of large and uniquely modified proteins. (2016) Nature Chemistry volume 8, pages 407-418; Amy E. Rabideau and Bradley Lether Pentelute*. Delivery of Non-Native Cargo into Mammalian Cells Using Anthrax Lethal Toxin. ACS Chem. (2016) Biol., 11(6) 1490-1501; and Weidmann et al., Copying Life: Synthesis of an Enzymatically Active Mirror-Image DNA-Ligase Made of D-Amino Acids. Cell Chemical Biology, (2019 May 16) 26(5); 616-619.

### EXAMPLES

The disclosure is further described in detail by reference to the following examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods.

### Example 1: Design and Manufacturing of the ACE2 ECD Fragment-IgG4 Fc Fusion Protein GL-4316

The ACE2 ECD fragment-IgG4 Fc fusion protein GL-4316 comprises from amino to carboxy terminus: a truncated ACE2 extracellular domain and an IgG4 Fc domain (FIG. 1). GL-4316 is produced as a single polypeptide chain comprising the amino acid sequence of SEQ ID NO: 50. In some embodiments, GL-4316 is produced as a single polypeptide chain comprising a signal sequence, which is cleaved from the mature protein (SEQ ID NO: 59). GL-4316 naturally forms a homodimer comprising two ACE2 extracellular domains and an IgG4 Fc domain (FIG. 1A). A more detailed schematic of the monomers and their association to form the functional forms of the fusion proteins describe herein are provided in FIG. 1B.

Manufacturing of GL-4316 generally comprises cell culture, harvest, purification, and formulation. Briefly, a selected mammalian host cell line (e.g., CHO Chinese hamster ovary cell line) was stably transfected with one or more expression vectors encoding GL-4316, comprising also a signal peptide that is cleaved from the mature secreted protein. Approximately 1920 cell clones were verified as single cells and then grown in a variety of media to select a clone and media yielding high viable cell density and GL-4316 expression levels titer. The expressed GL-4316 protein was then harvested from the culture supernatant and recovered from the supernatant using purification methods known in the art.

### Structural Analysis of GL-4316

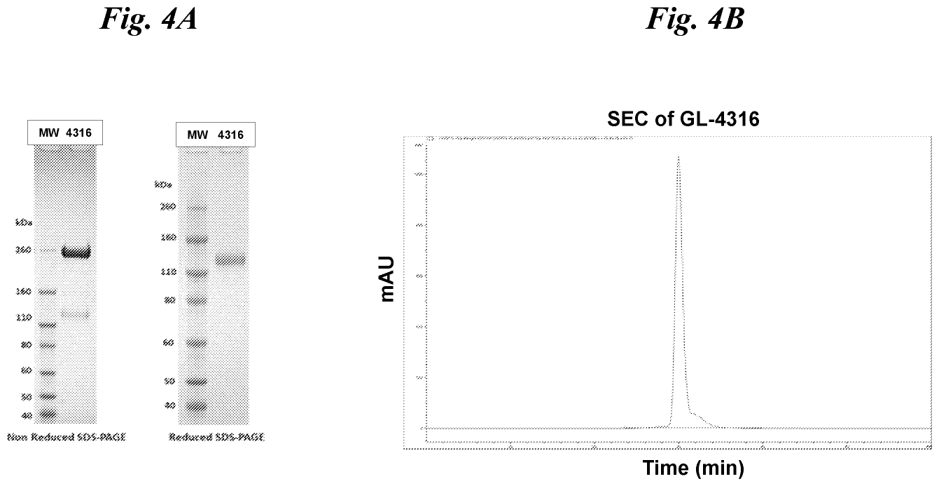

The structure of GL-4316 was assessed by non-reduced SDS-PAGE, reduced SDS-PAGE, and size-exclusion chromatography (SEC) after purification. Non-reduced SDS-PAGE showed an upper band below 260 kD that corresponded to the dimeric form of GL-4316 and a lower band at approximately 120 kD that represented the monomeric form of GL-4316 (FIG. 4A, left panel). Reduced SDS-PAGE showed a band at approximately 120 kD that corresponded to the monomeric form of GL-4316 (FIG. 4A, right panel).

SEC of GL-4316 revealed one major peak representing the dimeric form of GL-4316. The right shoulder of the major peak likely represented the monomeric form of GL-4316 (FIG. 4B).

### GL-4316 Binding to FcγR

Viruses have been associated with Antibody Dependent Enhancement (“ADE”), a process by which formation or delivery of antibodies can worsen the infectious process by antibody Fc-region binding to high and low affinity FcγRs. ADE has been implicated in the pathogenesis of Coronaviruses (Wan et al., Molecular mechanism for antibody dependent enhancement of coronavirus entry. J Virol. 2020; 94(5) pii: e02015-1; Liu et al., Anti-spike IgG causes severe acute lung injury by skewing macrophage responses during acute SARS-CoV infection. JCI Insight. 2019; 4(4) doi: 10.1172/jci.insight.123158. pii: 123158). GL-4316 comprises an IgG4 Fc domain specifically to reduce or eliminate binding to low affinity FcγRs in order to diminish the risk of ADE compared to a similar compound comprising an IgG1 Fc domain. Binding analysis was done using a ForteBio Octet Red system to confirm the reduced FcγR binding by GL-4316 compared to G001 (an IgG1 Fc domain alone). Binding was assessed in 1× kinetics binding buffer (ForteBio cat #18-1105). The concentrations of G001 and GL-4316 used were 200 μg/mL, 100 μg/mL, 50 μg/mL, 25 μg/mL, 12.5 μg/mL, and 6.25 μg/mL. Commercial recombinant His tagged receptors were loaded onto anti-His sensors from Forte bio (HIS1K cat #18-5121) at 5 μg/mL in 1×kinetics buffer for 300 sec and transferred to buffer for baseline measurement (60 s). On rate was measured for 300 s after transfer of sensor tip to kinetics buffer containing ligand. Off rate was measured for 600 s by transfer of sensor tip to kinetics buffer.

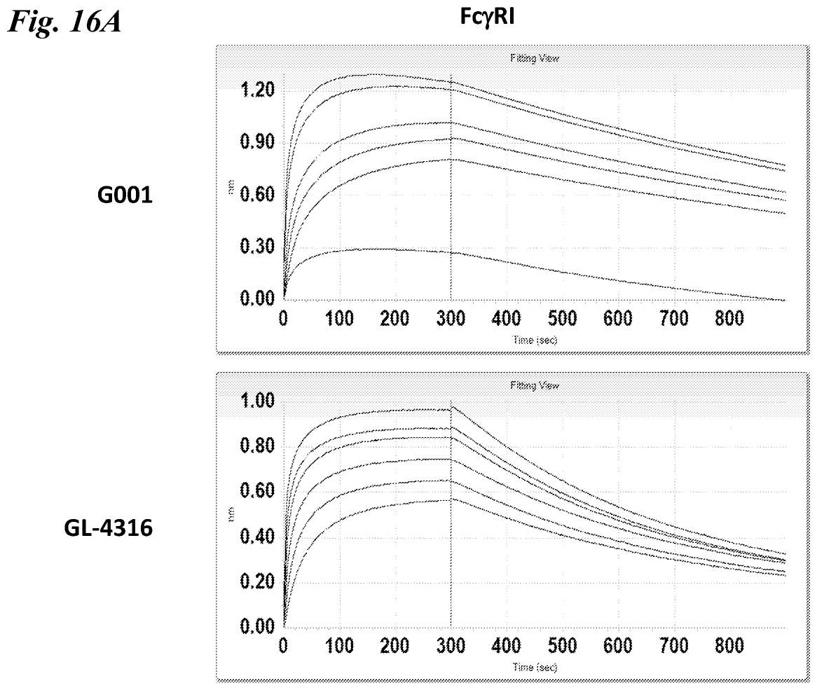

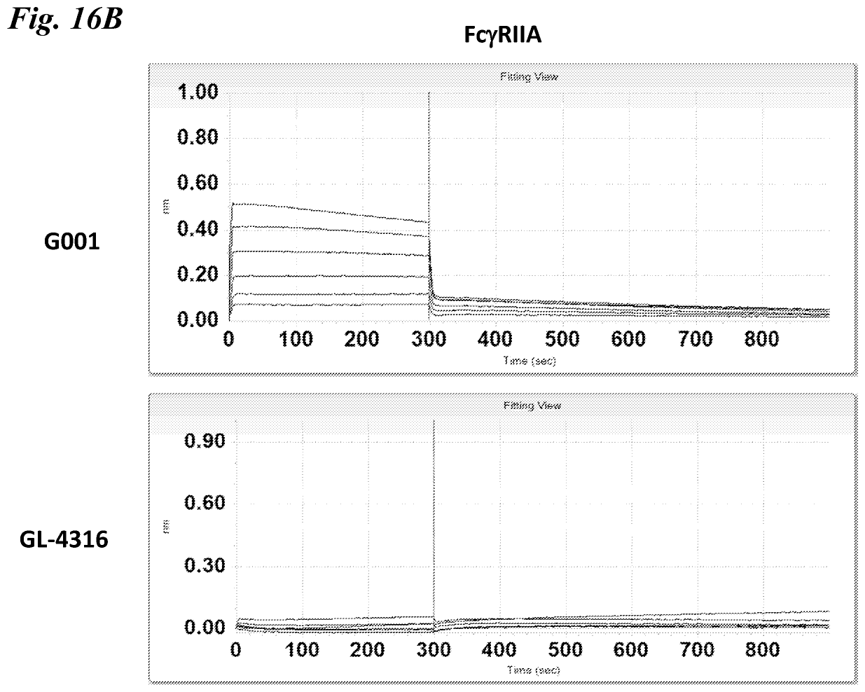

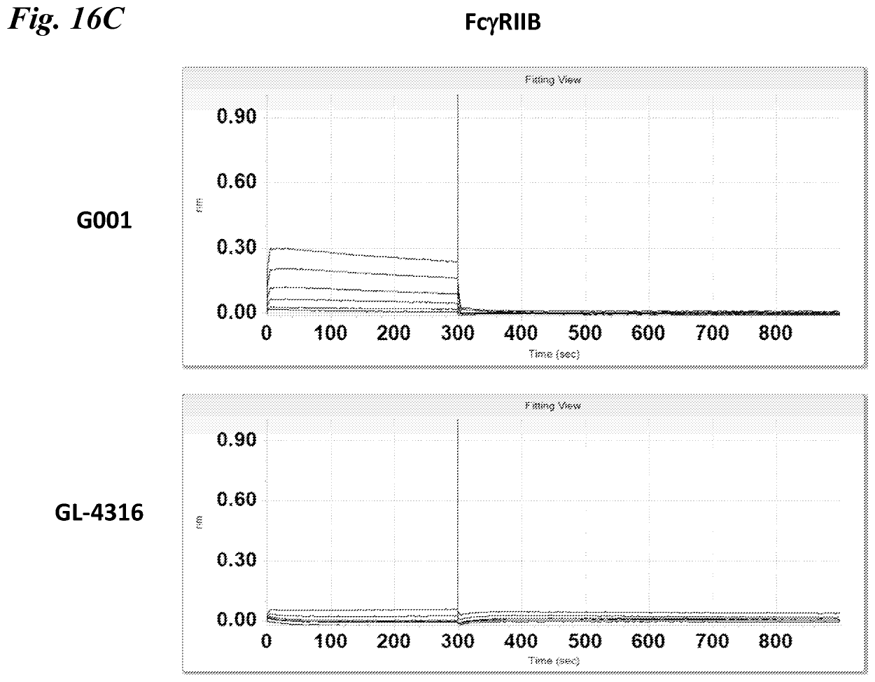

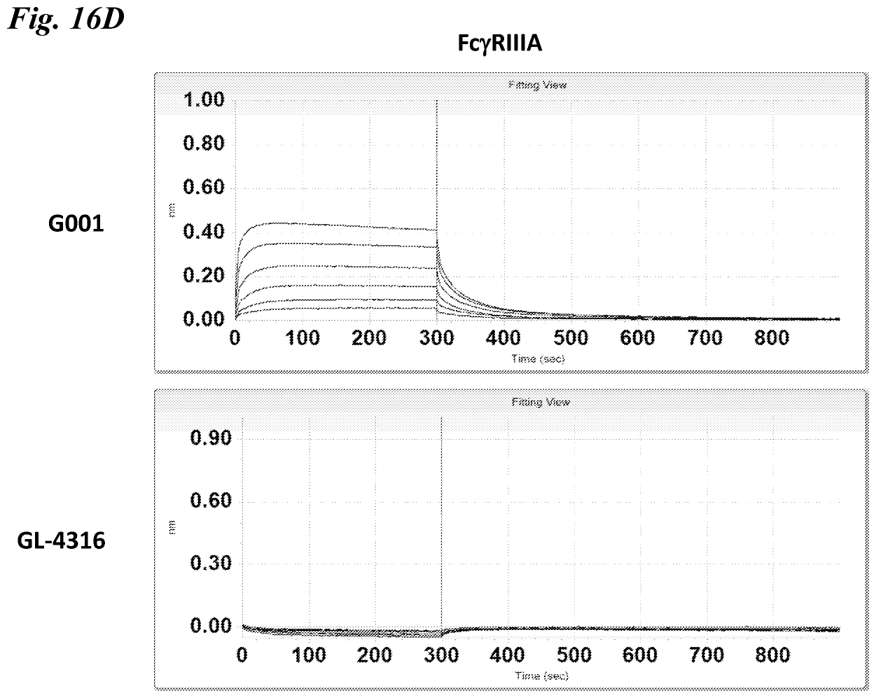

FIG. 16A illustrates the binding of G001 (a recombinant Fc control) and GL-4316 to FcγRI, FIG. 16B illustrates the binding of G001 (a recombinant Fc control) and GL-4316 to FcγRIIA. FIG. 16C illustrates the binding of G001 (a recombinant Fc control) and GL-4316 to FcγRIIB FIG. 16D illustrates the binding of G001 (a recombinant Fc control) and GL-4316 to FcγRIIIA Receptors used were from R&D System: FcγRI (cat #1257-FC). The absence of GL-4316 binding to low affinity Fc receptor demonstrates that the risk for ADE through the Fc domain of GL-4316 is low.

### ACE2 Enzymatic Activity

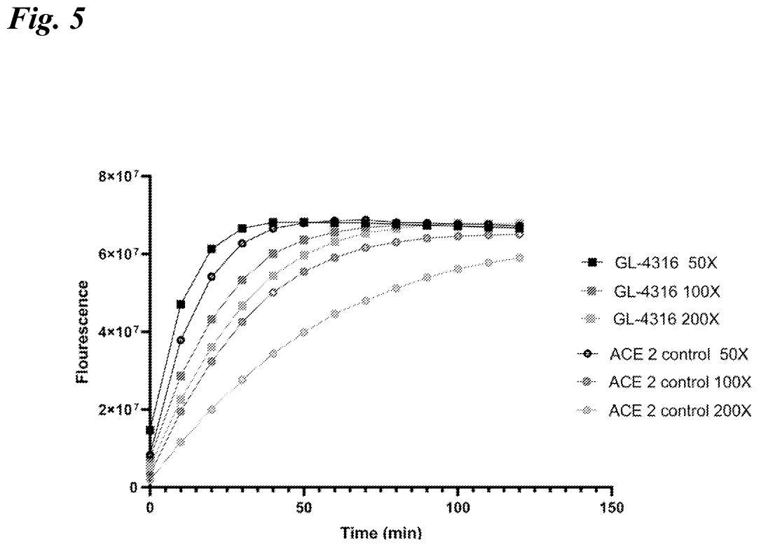

The enzymatic activity of GL-4316 was evaluated using an ACE2 Activity Assay Kit (Biovision, Inc., Cat. No. K897-100). Briefly, a synthetic MCA-based peptide substrate was incubated with a 50-fold, 100-fold, or 200-fold dilution of GL-4316 or ACE2 recombinant control (Sigma, Cat. No. SAE0064). Cleavage of the MCA-based peptide substrate by GL-4316 or the ACE2 recombinant control resulted in release of a fluorophore which was quantified using a fluorescence microplate reader (FIG. 5). The ACE2 enzymatic activity of GL-4316 was 2,168 pmol/min/μg and the ACE2 enzymatic activity of the ACE2 recombinant control was 1,860 pmol/min/μg.

### GL-4316 Binding to Neonatal Receptor (FcRn)

Transcytosis of Fc molecules via the neonatal receptor FcRn from circulation into peripheral tissues is well known in the art. GL-4316 was assessed for binding to FcRn compared to G001. Binding was done in 1×kinetics binding buffer (ForteBio cat #18-1105). Concentrations of G001 and GL-4316 used were 200 μg/mL, 100 μg/mL, 50 μg/mL, 25 μg/mL, 12.5 μg/mL, and 6.25 μg/mL. Binding analysis was done using a ForteBio Octet Red system. Commercial recombinant His tagged receptor was loaded onto anti-His sensors from Forte bio (HIS1K cat #18-5121) at 5 μg/mL in 1× kinetics buffer for 300 sec and transferred to buffer for baseline measurement (60 s). Receptors used were from R&D System: rhFcRN (cat #8639-FC).

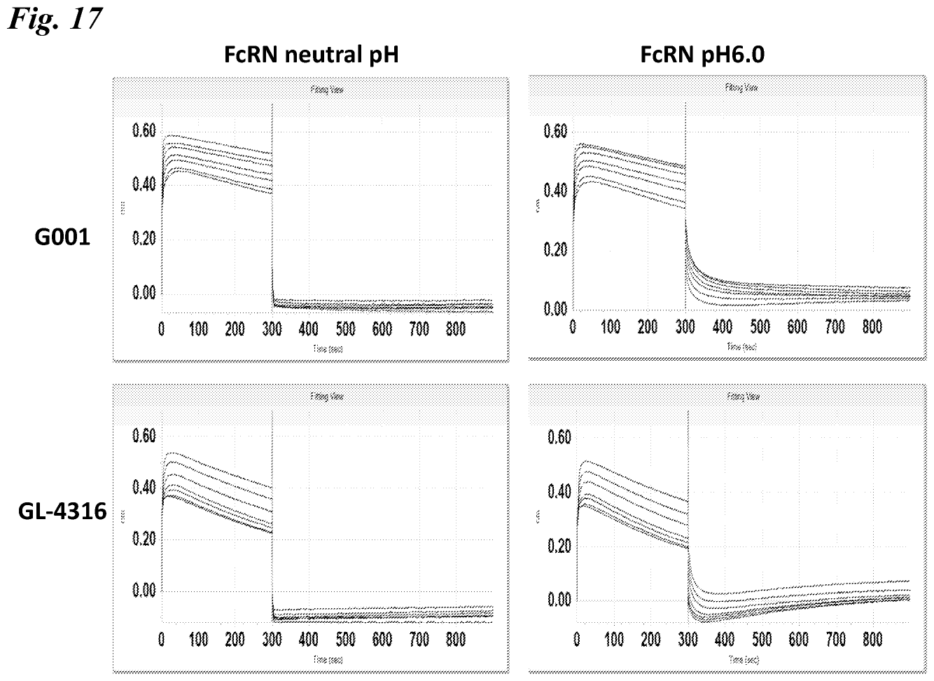

The binding curves of GL-4316 compared with G001 (IgG1 Fc) is provided in FIG. 17. In the first part of the experiment labelled “Neutral pH,” the sensor tip was loaded with FcRn protein at pH 7.4 followed by baseline at pH 6.0, association with protein at pH 6.0 and dissociation at pH 7.4. 300 s on time, 600 s off time. In the second part of the experiment labelled “pH 6.0,” the sensor tip was loaded with FcRn protein at pH 7.4 followed by baseline at pH 6.0, association with protein at pH 6.0 and dissociation at pH 6.0. 300 s on time, 600 s off time, analysis adjusted to baseline.

FIG. 17 demonstrates no functional difference in the dissociation rate of GL-4316 compared with G001, either at neutral pH or at pH 6.0.

### Example 2. Pharmacology/Toxicology of GL-4316

The in vivo pharmacology and safety of GL-4316 was evaluated in multiple animal models. A summary of the studies is described below.

### Rat Single Dose Study 1

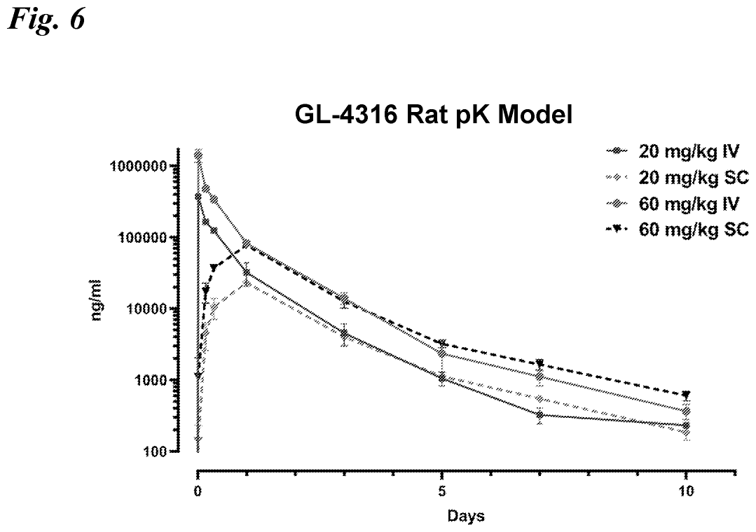

Rats were intravenously or subcutaneously administered 20 mg/kg or 60 mg/kg of GL-4316 and serum GL-4316 levels were measured overtime. The terminal half-life of GL-4316 was approximately 28 hours in rats. No significant differences in serum GL-4316 levels were observed between intravenous and subcutaneous administration after 24 hours (FIG. 6). The rats showed no adverse signs or symptoms upon administration of GL-4316.

### Rat Single Dose Study 2

Rats were intravenously administered 100 mg/kg of GL-4316 or a PBS control, and BALF and urine were collected in rats 24 hours following treatment. GL-4316 was measured in the BALF and urine of rats by ELISA and ACE2 enzymatic activity was measured using a Fluorometric Angiotensin II Converting Enzyme (ACE2) Activity Assay Kit (Biovision, CA). As shown in FIG. 7A, GL-4316 was recovered from the BALF of rats and exhibited enzymatic activity, indicating that GL-4316 successfully penetrated the peripheral tissue. GL-4316 was also detected in the urine, indicating that GL-4316 was excreted by the kidneys (FIG. 7B). The rats showed no adverse signs or symptoms upon administration of GL-4316.

### Cynomolgus Dose Range Study

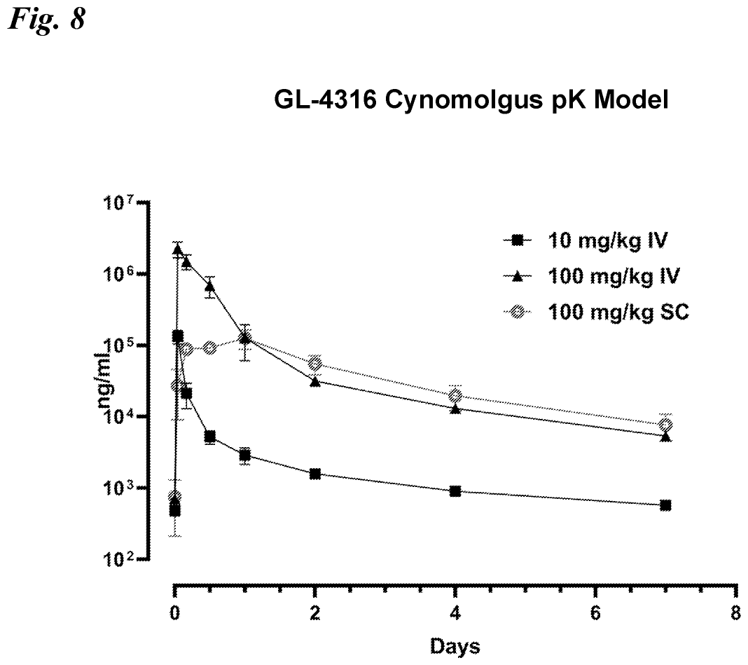

Cynomolgus monkeys were dosed with 10 mg/kg of GL-4316 intravenously (IV) or 100 mg/kg either intravenously or subcutaneously (SC) and serum was collected over time for assessment of GL-4316 levels by ELISA. As shown in FIG. 8, there was no difference starting at 24 hours in serum levels of GL-4316 between the intravenous and subcutaneous administration routes. These results also demonstrate a dose proportional increase of GL-4316. The terminal half-life of GL-4316 in cynomolgus monkeys based on the available data was estimated at approximately 42 to 89 hours.

### Example 3: GL-4316 Binds and Neutralizes SARS-CoV-2 In Vitro and In Vivo

The ability of GL-4316 to bind SARS-CoV-2 spike protein and neutralize viral infection was evaluated in vitro and in vivo.

GL-4316 binding of SARS-CoV-2 by ELISA

An ELISA was developed to assess SARS-CoV-2 binding by ACE2 ECD fragment-IgG4 Fc fusion protein (GL-4316). In brief, an ELISA plate was coated with SARS-CoV-2 S1 spike protein (Acrobiosystem, Cat No: SPN-CH52H8) at 2 μg/mL in PBS and reacted with purified GL-4316 protein at various concentration (0.78 ng/mL to 10 μg/mL). Bound GL-4316 protein was then detected using a polyclonal anti-human IgG Fc antibody (Thermo Scientific, Cat No: PAI-86854). GL-4316 demonstrated strong binding to the SARS-CoV-2 S1 spike protein with an EC₅₀ value of approximately 20 ng/mL (FIG. 9A). The dimeric form of GL-4316 surprisingly exhibited enhanced binding to SARS-CoV-2 S1 spike protein compared to the multimeric form of GL-4316 with an EC₅₀ value of 16 ng/mL and 611 ng/mL, respectively (FIG. 9B).

In further experiments, an ELISA plate was coated with SARS-CoV-2 D614 S1 spike protein (Sino Biological Cat #40591-VO8H) or the SARS-CoV-2 D614G S1 spike protein variant (Sino Biological Cat #40591-V08H3) at 0.5 μg/mL in PBS and reacted with purified GL-4316 protein at various concentration (0.78 ng/mL to 10 μg/mL). Bound GL-4316 protein was then detected using a polyclonal anti-human IgG Fc antibody (Thermo Scientific, Cat #PAI-86854). GL-4316 bound to the SARS-CoV-2 D614 and D614G spike 51 proteins equally at an EC₅₀ of 13 ng/mL and 12.5 ng/mL, respectively (FIG. 9C), demonstrating no decrease in binding for the most prevalent SARS-CoV-2 mutant in the U.S. as of June 2021.

GL-4316 binding of SARS-CoV-2 by Biolayer Interferometry Analysis

GL-4316 binding to SARS-CoV-2 S1 spike protein was analyzed on a ForteBio Octet Red96 instrument. In brief, sensor tips were loaded with SARS-CoV-2 S1 His-tagged spike protein expressed from human HEK293 cells (AMSbio, Cat No: AMS.S1N-052H3). Sensor tips were then reacted with purified GL-4316 protein and control protein at different concentrations and on and off rates were measured. The equilibrium dissociation constant (KD) was calculated by ForteBio Data Analysis 6.4 software module using measured on and off rates. Recombinant human IgG1 Fc (rFc) was used as control for binding analysis.

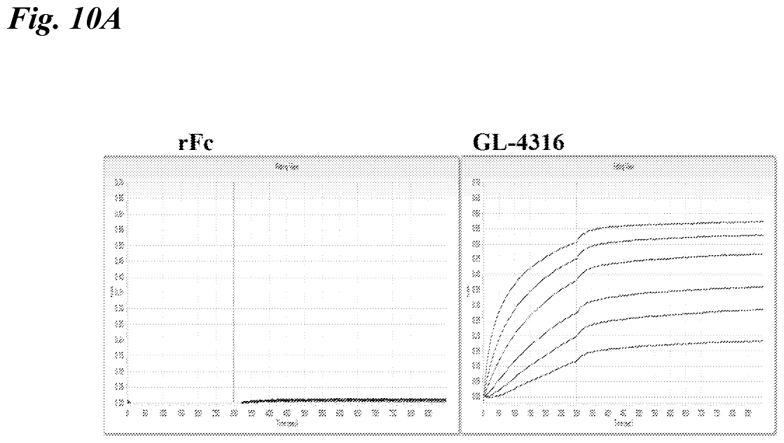

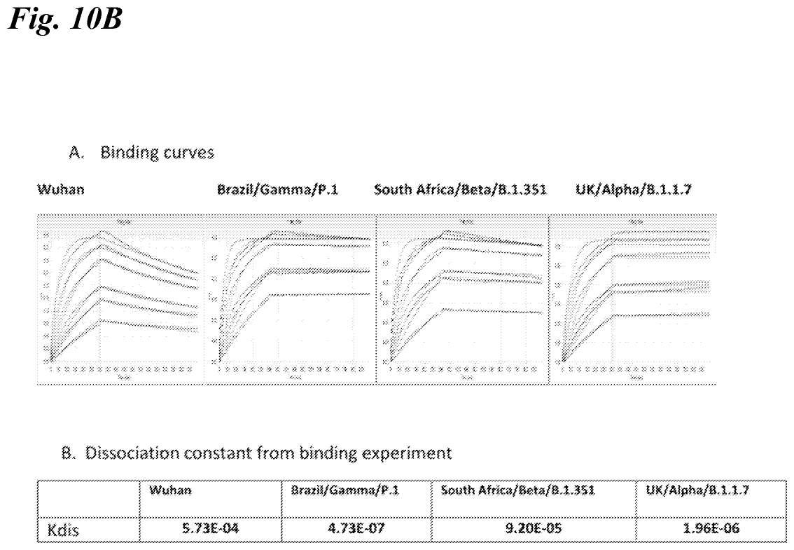

FIG. 10A shows binding curves of human IgG1 Fc (rFc) and GL-4316 to SARS-CoV-2 S1 protein as determined by biolayer interferometry. Table 5 shows kinetic parameters measured for the 51 protein interaction with GL-4316.

TABLE 5
Kinetics of S1 protein interaction with GL-4316

| | Kd | Kon | Kdis | Rmax | R² |
|---|---|---|---|---|---|
| rFC | NB | | | | |
| GL-4316 | 4.33E−10 | 2.83E+04 | 1.22E−05 | 0.5074 | 0.929 |

These results demonstrate a very low off rate for GL-4316 protein interaction with immobilized 51 protein, consistent with avid binding of the two ACE2 molecules of GL-4316 to spike protein.

Direct GL-4316 Binding to viral spike protein mutations by Biolayer Interferometry Analysis

GL-4316 binding to viral spike proteins was analyzed by biolayer interferometry. Binding was done in 1×kinetics binding buffer (ForteBio cat #18-1105). Concentrations of G001 and GL-4316 used were, 25 μg/mL, 12.5 μg/mL, 6.25 μg/mL, and 3.156 μg/mL, 1.578 ug/ml and 0.789 ug/ml

Binding analysis was done using a ForteBio Octet Red system. Commercial recombinant His tagged viral variants were loaded onto anti-His sensors from Forte bio (HIS1K cat #18-5121) in 1× kinetics buffer for 300 sec and transferred to buffer for baseline measurement (60 s). On rate was measured for 300 s after transfer of sensor tip to kinetics buffer containing ligand. Off rate was measured for 600 s by transfer of sensor tip to kinetics buffer.

The KD was calculated by ForteBio Data Analysis 6.4 software module using measured on and off rates and a 1:1 model fit. For KD calculations, an estimated average MW of 240 kD for GL-4316 was used. S proteins are from Acrobiosystems are derived from the SARS-CoV-2 spike protein parental sequence available at GenBank: QHD43416.1. Each of the derived sequences contain proline substitutions (F817P, A892P, A899P, A942P, K986P, V987P) and alanine substitutions (R683A and R685A) introduced to stabilize the trimeric prefusion state of SARS-CoV-2 S protein and abolish the furin cleavage site, respectively. This is referred to as the H9 Wuhan strain. Additional mutations are introduced into the Wuhan strain to represent additional variants of the spike protein which have become common in SARS-CoV-2 viruses from different regions throughout the pandemic. These recombinant proteins are expressed in HEK cells with a His-tag at the C terminus. The proteins tested were as follows:
Acrobiosystems cat #H9 (Wuhan) SPN-052H9— SEQ ID NO: 60.
Acrobiosystems cat #SPN-052H6 (H6), which is the B.1.1.17 (UK, WHO alpha) variant— SEQ ID NO: 61.
Acrobiosystems cat #SPN-052Hk (Hk) which is the B.1.351 (South Africa, WHO beta) variant— SEQ ID NO: 62.
Acrobiosystems cat #SPN-052Hg (Hg) which is the P.1 (Brazil, WHO gamma) variant—SEQ ID NO: 63.

Additional experiments with the B.1.617.2 (India, WHO delta variant, SEQ ID NO: 64) and the B.1.617.1 (India, WHO kappa variant, SEQ ID NO: 65) are performed and expected to show similar binding affinity for labeled ACE2. FIG. 10B summarizes the findings from this experiment. GL-4316 binds better to the novel SARS-CoV-2 variant S proteins than the S protein found in the original Wuhan strain as assessed by direct binding. While most of the binding parameters are the same for the different S proteins, the dissociation rate measured for the Wuhan strain S protein is significantly higher than the dissociation rate measured for the other tested S proteins (FIG. 10B) as can be seen in the binding curves (top) and the calculated Kdis (bottom). Importantly, these data demonstrate that GL-4316 binds the novel and clinically relevant mutated SARS-CoV-2 variants with greater potency than it binds the original Wuhan strain. This is in stark contrast to antibodies already used as COVID-19 therapeutics which have been demonstrated to have lost potency/binding to the mutated virus strains.

GL-4316 binding of SARS-CoV-2 by MSD Chemiluminescence

GL-4316 binding to viral spike protein variants was assessed with the MSD Mesoscale COVID-19 ACE2 neutralization kit (cat #K15440U) which contains several of the most pathogenic mutants in clinical circulation as of June 2021. The MSD neutralization kit quantitatively measures compounds that competitively inhibit the binding of labeled ACE2 to viral S proteins in the wells of a 96-well plate. The assay serves as an alternative to traditional cell-based neutralization assays.

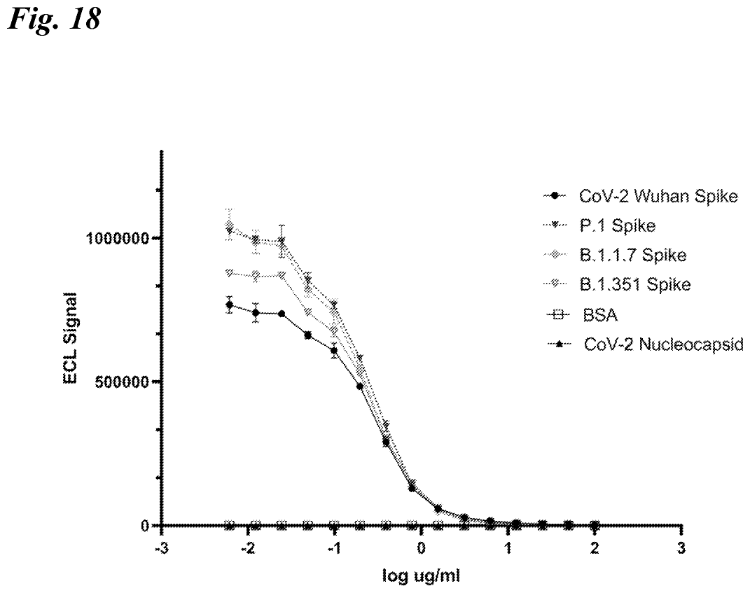

FIG. 18 demonstrates the results of the MSD neutralization binding experiment. SARS-CoV-2 spike protein from the original Wuhan virus (filled circles) binds to labeled ACE2 with an EC50=0.251 μg/mL in the presence of GL-4316. The SARS-CoV-2 B.1.1.7. (UK, WHO alpha variant) spike protein (filled diamonds) has an EC50=0.181 μg/mL. The SARS-CoV-2 B.1.351 (South Africa, WHO beta variant) spike protein (empty triangles) has an EC50=0.222 μg/mL. The SARS-CoV-2 P.1 (Brazil, WHO gamma variant) spike protein (filled triangles) has an EC50=0.207 μg/mL. BSA control is squares, COV-2 nucleocapsid protein control is filled triangles. Additional experiments with the B.1.617.2 (India, WHO delta variant, SEQ ID NO: 64 and the B.1.617.1 (India, WHO kappa variant, SEQ ID NO: 65) are performed and expected to show similar binding affinity for labeled ACE2.

No significant difference in EC50 was observed between the viral variants or the original SARS-CoV-2 spike indicating that GL-4316 outcompetes binding to the most significant clinical viral variants equally as well as it outcompetes binding to the original Wuhan SARS-CoV-2 spike protein.

In vitro Viral Neutralization with GL-4316

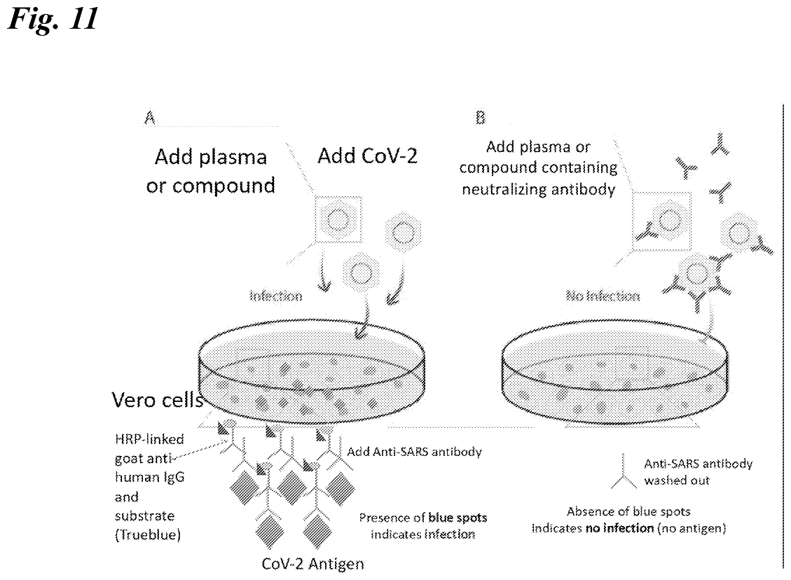

In vitro viral neutralization with GL-4316 was determined by a focus reduction neutralization assay (FRNA), and the read out was performed by using Enzyme Linked Immune Spot (ELISpot) as follows:
1. Serially diluted GL-4316 was incubated with SARS-CoV-2 (˜50-70 foci/well) for 1 h at 37° C. Positive control included a primate convalescent serum to SARS-related CoV-2.
2. Vero cells in 96-well plate were subsequently infected with the mixture for 1 h followed by addition of overlay media for foci assay and incubated for 3 days.
3. After 3 days incubation, the FRNA was performed using a monoclonal anti-SARS coronavirus recombinant human IgG1, clone CR3022 (BEI NR-52392), and foci were visualized and imaged using True Blue HRP substrate and ELISpot reader (CTL), respectively. FIG. 11 shows a schematic diagram of FRNA using ELISpot as a read-out.

The 50 and 90% effective concentration (EC_{50/90}) of GL-4316 required to inhibit viral protein expression was calculated by nonlinear regression analysis. The FRNA was repeated in two independent experiments and the percent inhibition values of SARS-CoV2 (relative to untreated control) were plotted in the graph (mean±SD).

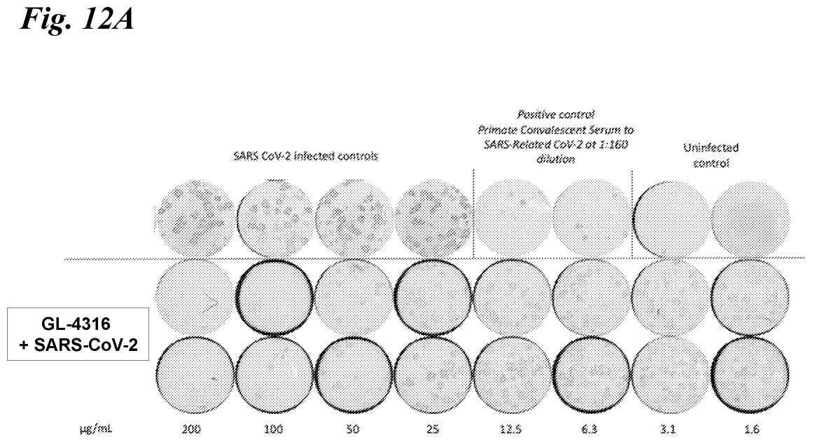

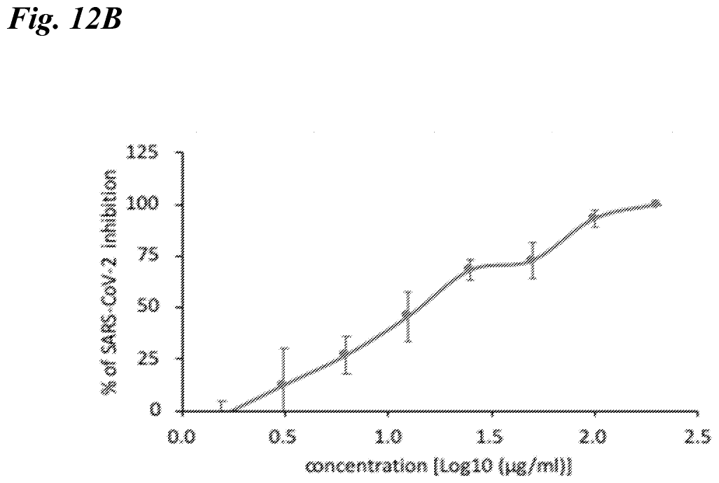

FIG. 12A shows representative panels of the FRNA in Vero cells infected with SARS CoV-2 virions. SARS-CoV-2 infected cells were either left untreated (positive control) or treated with GL-4316 or the convalescent serum control. These results show that entry of SARS-CoV-2 virions was inhibited by GL-4316 and by the positive convalescent serum control.

FIG. 12B shows the percent inhibition of SARS-CoV-2 fusion/entry in Vero cells. These results show that GL-4316 inhibited SARS-CoV-2 fusion/entry in Vero cells with EC_{50/90} values of 16.3±4.2 and 94.3±13.3 μg/mL. Notably, infectivity of SARS-CoV-2 was completely abolished by 200 μg/mL GL-4316, and at 100 μg/mL, exhibited 90.5% decrease in infectivity.

Cytotoxicity of GL-4316 was evaluated using an MTS cell proliferation assay. Briefly, Vero cells were treated with up to 200 μg/mL of GL-4316 or cyclohexamide (positive control) for four days. Vero cells treated with GL-4316 did not exhibit cytotoxicity at concentrations up to 200 μg/mL whereas Vero cells treated with cyclohexamide exhibited toxicity at an ICso of 0.2 (data not shown).

Viral Neutralization with GL-4316 In Vivo

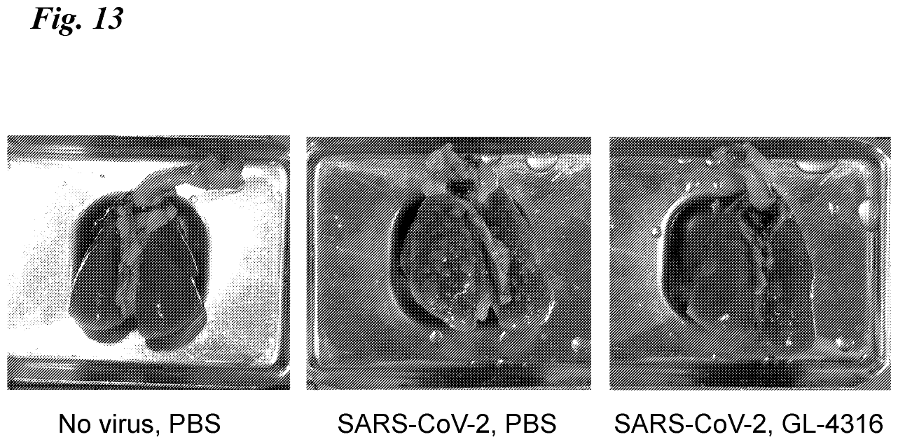

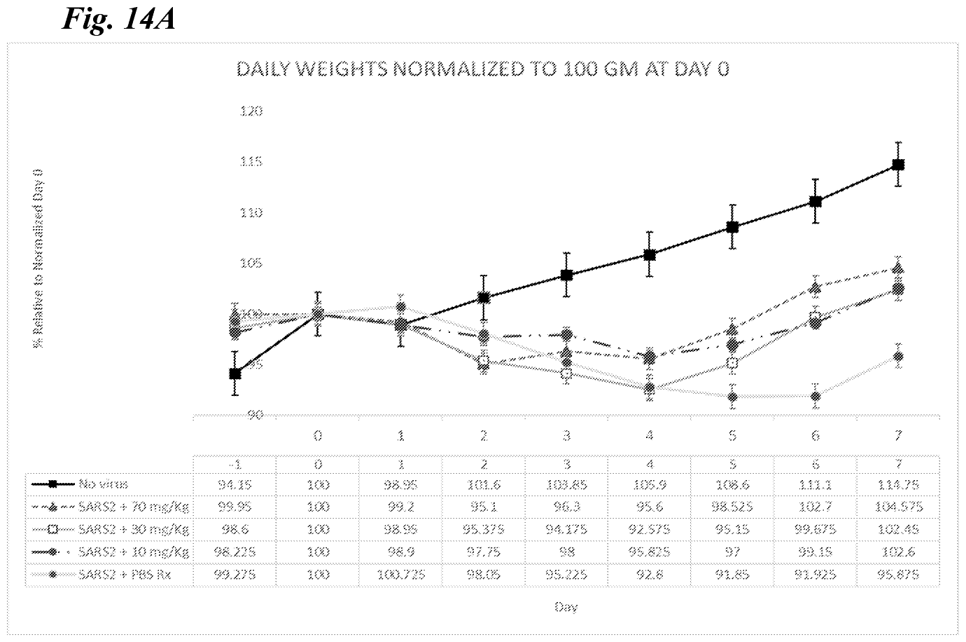

Golden Syrian Hamsters were intranasally infected with SARS-CoV-2 (2.0×10⁵ plaque forming units (PFUs)) and subcutaneously administered GL-4316 (50 mg/kg) or PBS. FIG. 13 shows the gross lung pathology of Golden Syrian hamsters treated with GL-4316 or PBS following SARS-CoV-2 infection compared to an uninfected PBS control. The lungs of GL-4316-treated hamsters demonstrate dramatically fewer inflammatory lesions on gross pathology which are larger in size than the PBS controls. SARS-CoV-2-infected Golden Syrian hamsters treated with GL-4316 also demonstrated approximately a one log lower viral load in the trachea compared to SARS-CoV-2-infected Golden Syrian hamsters treated with PBS (data not shown).

Protection from Weight Loss and Diminished Inflammation with GL-4316 In Vivo

In a second in vivo experiment, male Syrian hamsters were first subcutaneously administered GL-4316 (10, 30, or 70 mg/kg) or PBS at day −1. At day 0, hamsters were intranasally infected with SARS-CoV-2 (2.0×10⁴ PFUs) and subcutaneously administered GL-4316 or PBS at the same doses. Hamsters received a third subcutaneous administration of GL-4316 or PBS on day 2. Body weight was measured before and after SARS-CoV-2 administration (day −1 to day 7). Hamsters were followed for assessment of body weight daily for 7 days and then sacrificed to assess histopathologic changes of the lung.

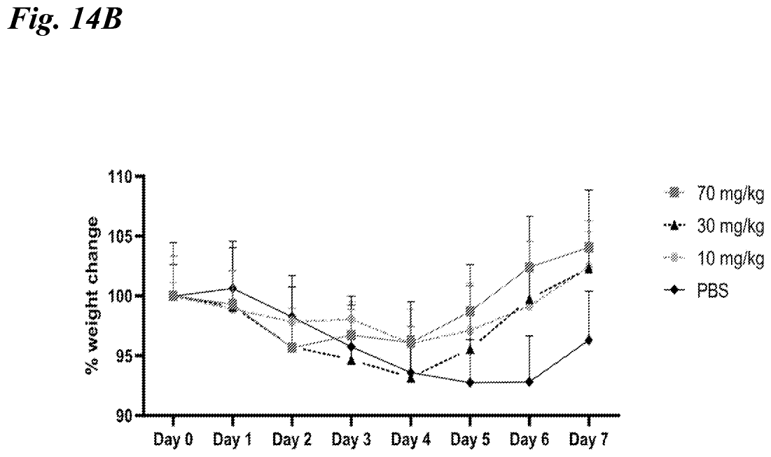

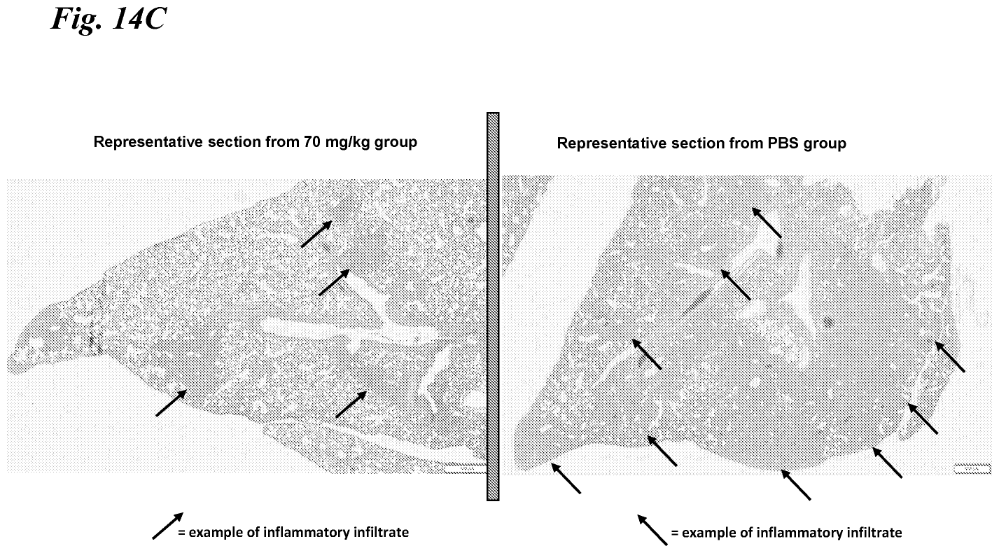

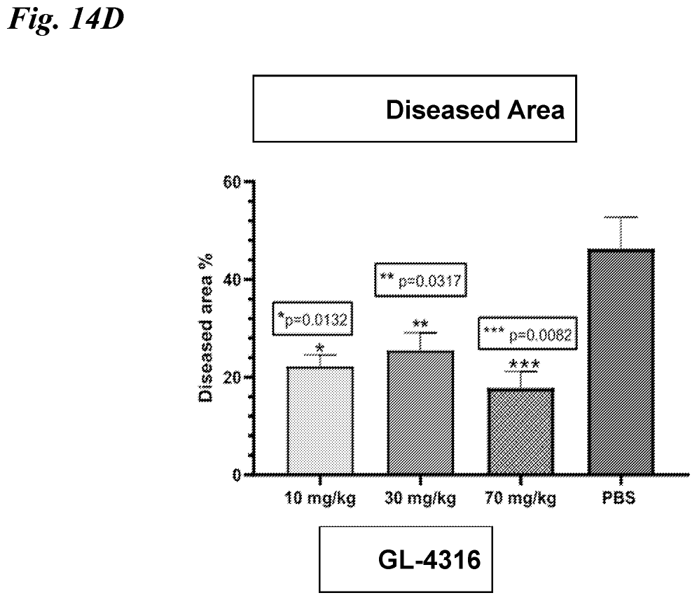

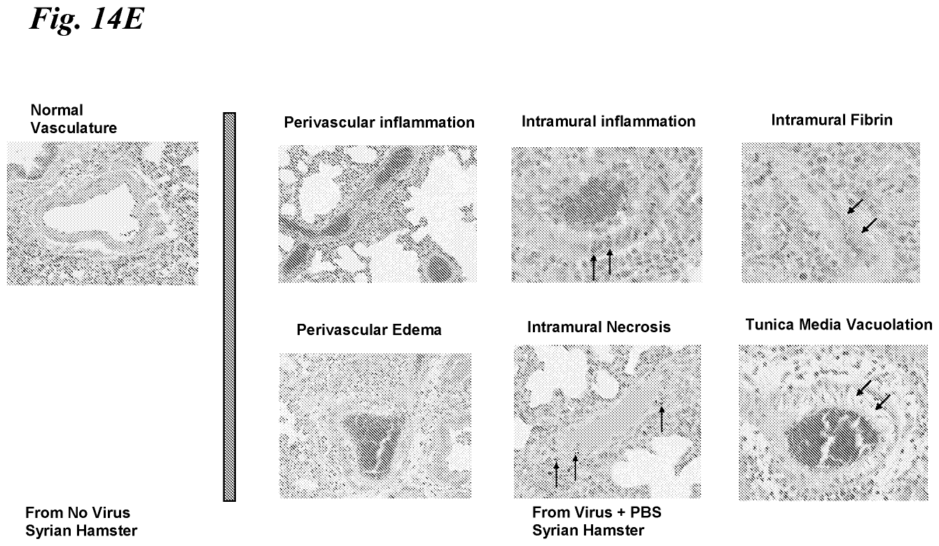

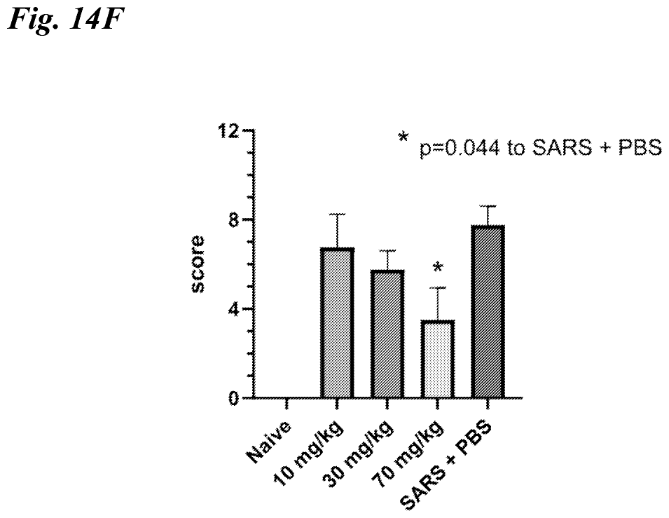

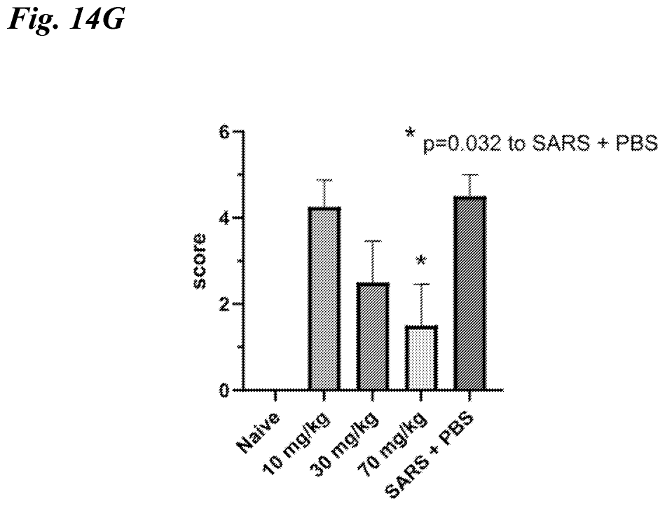

Individual animals' daily weights were normalized to the body weight at time zero in order to measure change from baseline. FIG. 14A and FIG. 14B demonstrate that hamsters infected with SARS-CoV-2 exhibited weight loss compared to uninfected hamsters. The highest dose of GL-4316 affords the greatest protection from weight loss. FIG. 14A and FIG. 14B demonstrate that SARS-CoV-2-infected hamsters treated with GL-4316 were partially protected from weight loss compared to SARS-CoV-2-infected hamsters treated with PBS to a degree comparable to reported protection from weight loss by currently marketed monoclonal antibodies directed against SARS-CoV-2 in prophylactic Syrian Hamster studies.

It is noted that the degree of protection from weight loss at day 7 with GL-4316 is approximately the same as the protection from weight loss at day 7 published for currently available monoclonal antibodies directed against SARS-CoV-2 spike protein despite those monoclonal antibodies consistently demonstrating artificially low EC50 values by several log orders in ex vivo neutralization assays relative to in vivo potency. The compounds of the present invention do not demonstrate this artifact, i.e. the neutralization assay data and the in vivo efficacy data demonstrate similar levels of potency.

FIG. 14C demonstrates representative lung sections from GL-4316-treated and placebo cohorts in this hamster model. As clearly shown, treatment with GL-4316 decreased the amount of lung inflammation and damage after SARS-CoV-2 infection. FIG. 14D demonstrates dramatic decrease in percent of lung area inflamed in the GL-4316-treated cohort compared with the PBS-treated cohort, as assessed by a histopathologist masked to treatment group. With 4 animals per cohort, the decrease in inflammation of the 70 mg/kg group relative to the PBS treated group was statistically significant with a p value=0.0082, indicating dramatic and consistent differences between treated and untreated hamsters. Further illustrative lung inflammation is provided in FIG. 14E showing normal lung vasculature (left) and exemplary inflammation-driven structural changes after virus infection (right).

FIG. 14F demonstrates significant suppression of SARS-CoV-2-induced lung vascular damage and vasculitis as quantitatively assessed by a histopathologist masked to treatment group. Six vascular pathology parameters were each scored 0-2 for each Syrian hamster (perivascular inflammation, perivascular edema, intramural inflammation, intramural necrosis, intramural fibrin deposition, and tunica media vacuolation). With 4 animals per cohort, the decrease in lung vascular damage and vasculitis at day 7 was dose-responsive with statistical significance (p value=0.044) for the 70 mg/kg group relative to the PBS treated group, indicating significant and consistent differences between treated and untreated hamsters. FIG. 14G demonstrates significant suppression of SARS-CoV-2-induced lung vascular intramural damage and vasculitis as quantitatively assessed by a histopathologist masked to treatment group. The three intravascular pathology parameters were each scored 0-2 for each Syrian hamster (intramural inflammation, intramural necrosis, intramural fibrin deposition). With 4 animals per cohort, the decrease in lung vascular intramural damage and vasculitis at day 7 was dose-responsive with statistical significance (p value=0.032) for the 70 mg/kg group relative to the PBS treated group, indicating significant and consistent differences between treated and untreated hamsters.

Golden Syrian Hamsters have become the standard model for assessment of drug efficacy in SARS-CoV-2 infection with weight loss and pathology as prime endpoints. Collectively, these data indicate that the ACE2-IgG4 fusion protein (GL-4316) functions as an anti-SARS CoV-2 target and protects animals from weight loss and lung inflammation.

### Example 4: ACE2-IgG1 Variants Effectively Bind SARS-CoV-2

Purpose: To design and test the ability of ACE2-IgG1 variants to bind SARS-CoV-2 spike protein in vitro.

### Design and Generation of Recombinant ACE2 IgG1 Variants

ACE2-IgG1 variants were designed to comprise a fragment of the ACE2 extracellular domain linked to an IgG1 Fc domain (FIG. 1). The ACE2 extracellular domain comprised one or more point mutations to examine how these mutations affected binding to viral spike protein. ACE2-IgG1 variants included a signal peptide at the N-terminus from a human immunoglobulin heavy chain. The signal peptide was not found in the mature protein.

### ACE2-IgG1 Variant Binding of SARS-CoV-2 by ELISA

An ELISA was developed to assess SARS-CoV-2 binding by ACE2-IgG1 and variants thereof. In brief, an ELISA plate was coated with SARS-CoV-2 S1 spike protein (Acrobiosystem, Cat No: SPN-CH52H8) at 2 μg/mL in PBS and reacted with purified ACE2-IgG fusion protein at various concentration. Bound ACE2-IgG1 fusion protein was then detected using a polyclonal anti-human IgG Fc antibody (Thermo Scientific, Cat. No: PAI-86854).

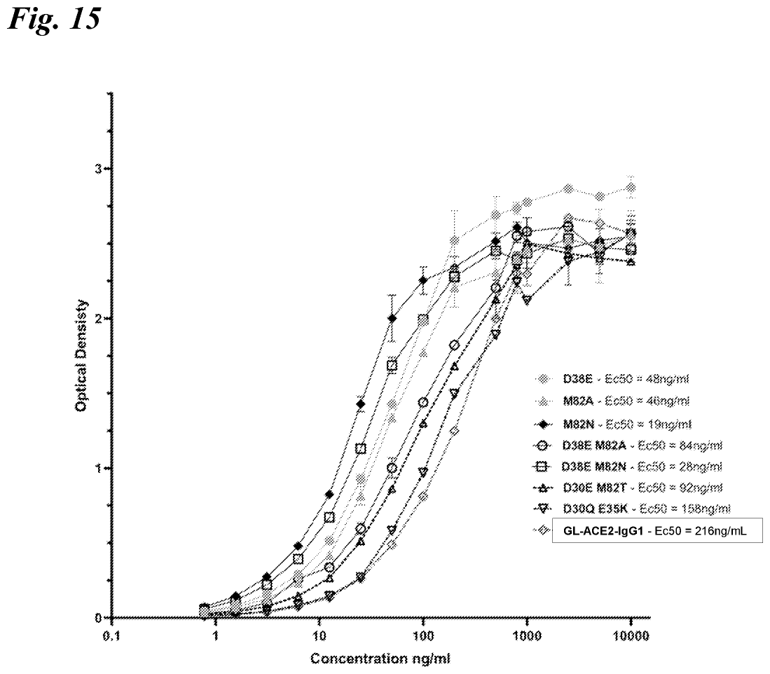

FIG. 15 shows binding of ACE2-IgG1 variants to SARS-CoV-2 by ELISA. The approximate EC₅₀ values of the ACE2-IgG1 fusion proteins range from approximately 20 ng/mL to 200 ng/mL.

These results demonstrate that ACE2-IgG1 fusion proteins comprising variants in the extracellular domain of ACE2 effectively bind to SARS-CoV-2 by ELISA. The ACE2-IgG1 fusion protein with the M82N point mutation had the lowest EC₅₀ value, suggesting that this variant exhibited the greatest potency to SARS-CoV-2.

### Example 5. ACE2-Fc Fusion Proteins for the Prevention of Symptomatic SARS-CoV-2

A subject suspected of having exposure to SARS-CoV-2, for example based on contact tracing, is administered a therapeutically effective amount of ACE2-Fc fusion protein (e.g., ACE2 ECD fragment-IgG4 Fc GL-4316 or variants thereof). The therapeutically effective amount is an amount sufficient to reduce the conversion rate of subjects tested positive for SARS-CoV-2 virus and/or the pathogenic effects of SARS-CoV-2 such as cough, fever, loss of taste or smell, requirement for oxygenation, requirement for hospitalization, requirement for intubation, requirement for Intensive Care Unit management, or mortality. Disease progression in the subject will be monitored. The subject may be tested to monitor the presence and/or abundance of SARS-CoV-2 viral load and the disease symptoms associated with SARS-CoV-2 infection before and after administration of the ACE2-Fc fusion protein.

### Example 6. ACE2-Fc Fusion Proteins for the Treatment of Asymptomatic or Early COVID-19 Secondary to SARS-CoV-2 Infection

A subject having or suspected of having SARS-CoV-2 will be tested to determine if they have been infected with SARS-CoV-2. The subject may be asymptomatic at the time of diagnosis or treatment or may have signs and symptoms consistent with early or mild COVID-19 disease, including but not limited to fever, non-productive cough, headache, diarrhea, taste or smell abnormalities, or shortness of breath. If the patient tests positive for SARS-CoV-2, or is clinically suspected of having SARS-CoV-2, a therapeutically effective amount of an ACE2-Fc fusion protein (e.g., ACE2 ECD fragment-IgG4 Fc GL-4316 or variants thereof) will be administered to the subject. The therapeutically effective amount is an amount sufficient to reduce the pathogenic effects of SARS-CoV-2. Disease progression in the subject will be monitored. The subject may be tested to monitor the presence and/or abundance of SARS-CoV-2 viral load and the disease symptoms associated with SARS-CoV-2 infection before and after administration of the ACE2-Fc fusion protein.

### Example 7. ACE2-Fc Fusion Proteins for the Treatment of Moderate or Severe COVID-19 Secondary to SARS-CoV-2 Infection

A subject who has been diagnosed as having SARS-CoV-2 may progress to moderate or severe disease or may be deemed to be at high risk for progression to advanced disease based on blood biomarkers, blood type, genetic markers and the like. Such patients may present with deterioration in the ratio of FiO2/PaO2, pneumonia, viral Acute Respiratory Distress Syndrome, seizures, cardiac arrhythmia or myocardial infarction, coronary artery aneurysms, acute kidney disease, and similar systemic manifestations of disease that may require ventilation, dialysis, and other interventions. While SARS-CoV-2 viral load normally is decreasing by day 10 after initial symptom onset, these patients may maintain an elevated viral load. In this case, a therapeutically effective amount of ACE2-Fc fusion protein (e.g., ACE2 ECD fragment-IgG4 Fc GL-4316 or variants thereof) will be administered to the subject. The therapeutically effective amount is an amount sufficient to reduce the pathogenic effects of SARS-CoV-2. Disease progression in the subject will be monitored. The subject may be tested to monitor the presence and/or abundance of SARS-CoV-2 viral load, before and after administration of the ACE2-Fc fusion protein.

### Example 8. ACE2-Fc Fusion Proteins for the Treatment of Chronic COVID Syndrome

A subject who has been diagnosed as having SARS-CoV-2 may progress to chronic COVID syndrome or may be deemed to be at high risk for progression to chronic COVID syndrome based on blood biomarkers (such as low vitamin D level), blood type (such as Type A blood), genetic markers (such as lower functioning or lower expression phenotypes and genetic variants of ACE2) and the like. Such patients may present with fatigue, shortness of breath, cough, joint pain, chest pain, brain fog, depression, muscle pain, headache, intermittent fever, heart palpitations, loss of smell and taste, insomnia, rash, hair loss, acute kidney injury, reduced lung function, and anxiety. To treat subjects with chronic COVID syndrome, a therapeutically effective amount of ACE2-Fc fusion protein (e.g., ACE2 ECD fragment-IgG4 Fc GL-4316 or variants thereof) will be administered to the subject. The therapeutically effective amount is an amount sufficient to reduce or eliminate the symptoms associated with chronic COVID syndrome. Disease symptoms in the subject with chronic COVID syndrome will be monitored before and after administration of the ACE2-Fc fusion protein.

### Example 9. Stockpiling Drug for Future Pandemics

Both SARS-CoV-1 and SARS-CoV-2 mediate pathological effects primarily through binding of viral spike protein to host cell receptors, primarily ACE2 but also other receptors including CD147 and NRP1. Therefore, it is apparent that future, potentially pandemic, coronaviruses may utilize the spike protein to enter host cells. To be prepared for future pandemics, a therapeutically effective amount of ACE2-Fc fusion protein (e.g., ACE2 ECD fragment-IgG4 Fc GL-4316 or variants thereof) will be stockpiled to be administered to a subject who becomes infected with a pathogenic microorganism such as a coronavirus that binds the ACE2 receptor.

### Example 10. ACE2-Fc Fusion Proteins for the Treatment of Pulmonary Hypertension

A subject having or suspected of having pulmonary hypertension will be tested to determine if they have the disease. If the patient tests positive, a therapeutically effective amount of ACE2-Fc fusion protein (e.g., ACE2 ECD fragment-IgG4 Fc GL-4316 or variants thereof) will be administered to the subject. The therapeutically effective amount is an amount sufficient to reduce the pathogenic effects of pulmonary hypertension.

### Example 11. ACE2-Fc Fusion Proteins for the Treatment of Acute Lung Injury

A subject having or suspected of having acute lung injury will be tested to determine if they have acute lung injury. Such acute lung injury can occur, for example, as a result of exposure to influenza virus, SARS-CoV-1, SARS-CoV-2, or toxins. If the patient tests positive, a therapeutically effective amount of ACE2-Fc fusion protein (e.g., ACE2 ECD fragment-IgG4 Fc GL-4316 or variants thereof) will be administered to the subject. The therapeutically effective amount is an amount sufficient to reduce the pathogenic effects of acute lung injury.

### Example 12. ACE2-Fc Fusion Proteins for the Treatment of Endometriosis

A subject having or suspected of having endometriosis will be tested to determine if they have the disease. If the patient tests positive, a therapeutically effective amount of ACE2-Fc fusion protein (e.g., ACE2 ECD fragment-IgG4 Fc GL-4316 or variants thereof) will be administered to the subject. The therapeutically effective amount is an amount sufficient to reduce the pathogenic effects of endometriosis or the pain associated with endometriosis.

### Example 13. ACE2-Fc Fusion Proteins for the Treatment of Sarcoidosis

A subject having or suspected of having sarcoidosis will be tested to determine if they have the disease. If the patient tests positive, a therapeutically effective amount of ACE2-Fc fusion protein (e.g., ACE2 ECD fragment-IgG4 Fc GL-4316 or variants thereof) will be administered to the subject. The therapeutically effective amount is an amount sufficient to reduce the pathogenic effects of sarcoidosis or the shortness of breath, eye pain or dryness, blurred vision, rash, cough or weight loss associated with sarcoidosis.

### Example 14. Rodent Model Characterized by Decreased ACE2 Levels and Mortality

While none of the currently available animal models of SARS1 or SARS-CoV-2 has demonstrated the acquired ACE2 deficiency that likely characterizes human COVID-19, in experimental mouse models infection with highly pathogenic avian influenza A H5N1 virus results in downregulation of ACE2 expression in the lung and increased serum angiotensin II levels. Genetic inactivation of ACE2 causes severe lung injury in H5N1-challenged mice, confirming a role of ACE2 in H5N1-induced lung pathologies. Administration of recombinant human ACE2 ameliorates avian influenza H5N1 virus-induced lung injury in mice (Zou Z et al. Angiotensin-converting enzyme 2 protects from lethal avian influenza A H5N1 infections. Nat Comm. 2013). This experiment will demonstrate in a mouse model characterized by both decreased ACE2 expression and mortality that GL-4316 decreases mortality by replenishing the influenza-induced ACE2 enzyme deficiency. For example, the strain of mice may be BL6 and the virus associated with decreased ACE2 expression may be H5N1-PR7. ACE2 expression may be measured by mRNA expressions levels (e.g. NanoString), by protein expression levels (e.g. ELISA), or other standard methods. In this experiment, GL-4316 will improve mortality compared with untreated control, demonstrating the anti-inflammatory efficacy of the compounds of this invention.

### Example 15. Healthy Individuals Protected from Developing COVID-19

The many evolving mutations of SARS-CoV-2 risks vaccines and other antiviral drugs such as monoclonal antibody combinations of becoming less effective. In order for society to return to normal including people returning to work offices, eating out at restaurants or bars, or gathering with family or with strangers in settings such as houses of worship or movie theaters, requires a prophylactic agent that will adequately protect against inadvertent inoculation of SARS-CoV-2 and onset of COVID-19. Administration of recombinant human ACE2 will bind and neutralize any mutant version of SARS-CoV-2 that retains the ability to bind human ACE2. GL-4316 improves on recombinant human ACE2 by providing extended half-life and increased ability to penetrate tissue via the FcRn receptor and because of its truncated ACE2 ECD. The human nasal passage and retropharynx express high levels of ACE2. This experiment will demonstrate in humans that intranasal spray, mouthwash, or an inhaler with GL-4316 decreases COVID-19 morbidity and mortality by providing ACE2 to directly bind and neutralize all mutated forms of SARS-CoV-2. Healthy individuals can select the times of administration that best suits their needs for protection. When going to work, or to a public indoor space such as a restaurant, the subject either inhales GL-4316, sprays GL-4316 intranasally, washes the retropharynx with GL-4316, or any combination thereof. GL-4316 remains available to bind and neutralize virus for a period of at least 1 hour. The subject may repeat dosing as needed for ongoing exposure. When alone with family at night or on a weekend, the subject may opt not to administer drug because of low risk.

### Example 16. Selecting Subjects for Treatment with an ACE2-Fc Fusion Protein

Experiments are performed to assess Ang II, des-arg-9-bradykinin, and Ang 1-7 as biomarkers of inflammation and selection of subjects for treatment with the ACE2-Fc fusion proteins described herein. Briefly, plasma is collected from subjects suffering from an inflammatory disease or condition, including viral infection by SARS-CoV-2 or influenza, sarcoidosis, endometriosis, acute lung injury, pulmonary hypertension, and chronic COVID syndrome. Levels of Ang II, des-arg-9-bradykinin, and Ang 1-7 are determined from the collected plasma samples. Subjects with levels of Ang II or des-arg-9-bradykinin that exceed the normal range are selected for treatment with an ACE2-Fc fusion protein such as GL-4316. Additionally, the ratio of Ang II to Ang 1-7 is assessed. Subjects with an increased Ang II/Ang 1-7 ratio, either by an increase in Ang II or a decrease in Ang 1-7 or both are further selected for treatment. Subjects with increased Ang II, increased des-arg-9-bradykinin, decreased Ang 1-7, or increased ratio of Ang II:Ang 1-7 may be additionally followed with these same tests repetitively to assess the effect of therapeutic treatment with an ACE2-Fc fusion protein such as GL-4316 and the need for additional treatment until the level of Ang II, des-arg-9-bradykinin, and/or the ratio of Ang II:Ang 1-7 approaches or reaches normal values as determined by population normal values.

### Further Embodiments of the Invention

Other subject matter contemplated by the present disclosure is set out in the following numbered embodiments:

### Embodiment 1

An angiotensin converting enzyme 2 (ACE2) Fc fusion protein comprising: an ACE2 extracellular domain or fragment thereof; and one or more Fc domains.

### Embodiment 2

A homodimeric angiotensin converting enzyme 2 (ACE2) Fc fusion protein comprising a first and a second polypeptide monomer, wherein each monomer comprises an ACE2 extracellular domain or fragment thereof; and one or more Fc domain monomers, wherein the one or more Fc domain monomers in the first polypeptide monomer associate with the one or more Fc domain monomers in the second polypeptide monomer to form one or more Fc domains.

### Embodiment 3

The ACE2-Fc fusion protein of Embodiment 1 or 2, wherein the one or more Fc domains demonstrate reduced binding to one or more low affinity Fe□ receptors compared to a wild type IgG1 Fc domain.

### Embodiment 4

The ACE2-Fc fusion protein of any one of Embodiments 1-3, wherein the one or more Fc domains are IgG4.

### Embodiment 5

The ACE2-Fc fusion protein of any one of Embodiments 1-3, wherein the one or more Fc domains are IgG1 or IgG3 Fc domains that have been mutated to reduce binding to one or more low affinity Fe□ receptors.

### Embodiment 6

The ACE2-Fc fusion protein of any one of Embodiments 1-5, wherein the ACE2 extracellular domain comprises an amino acid sequence that is at least 90%, 91%, 92%, 93%, 94%, or 95% identical to SEQ ID NO: 6.

### Embodiment 7

An angiotensin converting enzyme 2 (ACE2) Fc fusion protein comprising: an ACE2 extracellular domain fragment; and one or more Fc domains, wherein the one or more Fc domains demonstrate reduced binding to one or more low affinity Fe□ receptors compared to a wild type IgG1 Fc domain.

### Embodiment 8

A dimeric angiotensin converting enzyme 2 (ACE2) Fc fusion protein a first and a second polypeptide monomers, wherein each monomer comprises: an ACE2 extracellular domain fragment; and one or more Fc domain monomers, wherein the one or more Fc domain monomers in the first polypeptide monomer associate with the one or more Fc domain monomers in the second polypeptide monomer to form one or more Fc domains, and wherein the one or more Fc domains demonstrate reduced binding to one or more low affinity Fey receptors compared to a wild type IgG1 Fc domain.

### Embodiment 9

The ACE2-Fc fusion protein of any one of Embodiments 1-8, wherein the ACE2 extracellular domain fragment comprises an amino acid sequence that is at least 90%, 91%, 92%, 93%, 94%, or 95% identical to of SEQ ID NO: 8.

### Embodiment 10

A dimeric angiotensin converting enzyme 2 (ACE2) Fc fusion protein comprising a first and a second polypeptide chain, wherein the first polypeptide chain comprises an ACE2 extracellular domain or ligand-binding fragment thereof, and a first Fc domain monomer polypeptide chain; and the second polypeptide chain comprises an Fc domain monomer polypeptide chain.

### Embodiment 11

The ACE2-Fc fusion protein of Embodiment 10, wherein the second polypeptide chain further comprises an ACE2 extracellular domain or ligand-binding fragment thereof.

### Embodiment 12

The ACE2-Fc fusion protein of Embodiment 10 or 11, wherein the first Fc domain monomer polypeptide chain and the second Fc domain monomer polypeptide chain of the second polypeptide chain form an Fc domain.

### Embodiment 13

The ACE2-Fc fusion protein of embodiment 12, wherein the ACE2 Fc fusion protein is a homodimer.

### Embodiment 14

The ACE2-Fc fusion protein of any one of Embodiments 1-13, wherein the ACE2 extracellular domain is a ligand-binding fragment thereof.

### Embodiment 15

The ACE2-Fc fusion protein of any one of Embodiments 10-14, wherein the ACE2 extracellular domain fragment comprises an amino acid sequence that is at least 90%, 91%, 92%, 93%, 94%, or 95% identical to of SEQ ID NO: 8.

### Embodiment 16

The ACE2-Fc fusion protein of any one of Embodiments 1-15, wherein the ACE2 extracellular domain or fragment thereof further comprises a signal peptide of SEQ ID NO: 2.

### Embodiment 17

The ACE2-Fc fusion protein of Embodiment 15, wherein the signal peptide is cleaved from the mature protein.

### Embodiment 18

The ACE2-Fc fusion protein of any one of Embodiments 1-17, wherein the ACE2 extracellular domain or fragment thereof comprises one or more point mutations.

### Embodiment 19

The ACE2-Fc fusion protein of Embodiment 18, wherein the ACE2 extracellular domain or fragment thereof comprises a point mutation at position 82 of SEQ ID NO: 5 or SEQ ID NO: 7.

### Embodiment 20

The ACE2-Fc fusion protein of Embodiment 19, wherein the ACE2 extracellular domain or fragment thereof comprises the point mutation M82A, M82D, M82N, or M82S.

### Embodiment 21

The ACE2-Fc fusion protein of Embodiment 18, wherein the ACE2 extracellular domain or fragment thereof comprises a point mutation at position 30 of SEQ ID NO: 5 or SEQ ID NO: 7.

### Embodiment 22

The ACE2-Fc fusion protein of Embodiment 21, wherein the ACE2 extracellular domain or fragment thereof comprises the point mutation D30E or D30Q.

### Embodiment 23

The ACE2-Fc fusion protein of Embodiment 18, wherein the ACE2 extracellular domain or fragment thereof comprises a point mutation at position 31, 34, and/or 38 of SEQ ID NO: 5 or SEQ ID NO: 7.

### Embodiment 24

The ACE2-Fc fusion protein of Embodiment 23, wherein the ACE2 extracellular domain or fragment thereof comprises a K31T point mutation.

### Embodiment 25

The ACE2-Fc fusion protein of Embodiment 23, wherein the ACE2 extracellular domain or fragment thereof comprises a H34Q point mutation.

### Embodiment 26

The ACE2-Fc fusion protein of Embodiment 23, wherein the ACE2 extracellular domain or fragment thereof comprises a D38E point mutation.

### Embodiment 27

The ACE2-Fc fusion protein of Embodiment 18, wherein the ACE2 extracellular domain or fragment thereof comprises one or more point mutations selected from the group consisting of D30E, K31T, H34Q, and D38E.

### Embodiment 28

The ACE2-Fc fusion protein of Embodiment 18, wherein the ACE2 extracellular domain or fragment thereof comprises a point mutation at position 139 of SEQ ID NO: 5 or SEQ ID NO: 7.

### Embodiment 29

The ACE2-Fc fusion protein of Embodiment 28, wherein the ACE2 extracellular domain or fragment thereof comprises the point mutation Q139A, Q139S, or Q139V.

### Embodiment 30

The ACE2-Fc fusion protein of Embodiment 18, wherein the ACE2 extracellular domain or fragment thereof comprises a point mutation at position 175 of SEQ ID NO: 5 or SEQ ID NO: 7.

### Embodiment 31

The ACE2-Fc fusion protein of Embodiment 30, wherein the ACE2 extracellular domain or fragment thereof comprises the point mutation Q175A, Q175S, or Q175V.

### Embodiment 32

The ACE2-Fc fusion protein of Embodiment 18, wherein the ACE2 extracellular domain or fragment thereof comprises a point mutation at position 374 and/or position 378 of SEQ ID NO: 5 or SEQ ID NO: 7.

### Embodiment 33

The ACE2-Fc fusion protein of Embodiment 32, wherein the ACE2 extracellular domain or fragment thereof comprises the point mutation H374S, H374A, or H374V and/or H378S, H378A, or H378V.

### Embodiment 34

The ACE2-Fc fusion protein of Embodiment 18, wherein the ACE2 extracellular domain or fragment thereof comprises the point mutations M82N, Q139A, H374S, and H378S.

### Embodiment 35

The ACE2-Fc fusion protein of any one of Embodiments 1-3 or 5-34, wherein the Fc domain is an IgG1 Fc domain.

### Embodiment 36

The ACE2-Fc fusion protein of Embodiment 35, wherein the IgG1 Fc domain comprises an IgG1 hinge, an IgG1 CH2 domain, and an IgG1 CH3 domain.

### Embodiment 37

The ACE2-Fc fusion protein of Embodiment 36, wherein the IgG1 Fc domain comprises an amino acid sequence of SEQ ID NO: 39.

### Embodiment 38

The ACE2-Fc fusion protein of any one of Embodiments 1-4 or 6-34, wherein the Fc domain is an IgG4 Fc domain.

### Embodiment 39

The ACE2-Fc fusion protein of Embodiment 38, wherein the IgG4 Fc domain comprises an IgG4 hinge, an IgG4 CH2 domain, and an IgG4 CH3 domain.

### Embodiment 40

The ACE2-Fc fusion protein of Embodiment 39, wherein the IgG4 Fc domain comprises an amino acid sequence of SEQ ID NO: 42.

### Embodiment 41

The ACE2-Fc fusion protein of any one of Embodiments 1-40, further comprising a signal peptide, wherein the signal peptide comprises an amino acid sequence of SEQ ID NO: 2.

### Embodiment 42

The ACE2-Fc fusion protein of Embodiment 41, wherein the signal peptide is cleaved from the ACE2-Fc fusion protein.

### Embodiment 43

The ACE2-Fc fusion protein of Embodiment 42, wherein the signal peptide is cleaved between amino acid positions 17 and 18 of the ACE2-Fc fusion protein comprising the amino acid sequence selected from the group consisting of SEQ ID NO: 43-50.

### Embodiment 44

The ACE2-Fc fusion protein of Embodiment 42, wherein the signal peptide is cleaved between amino acid positions 19 and 20 of the ACE2-Fc fusion protein comprising the amino acid sequence selected from the group consisting of SEQ ID NO: 43-50.

### Embodiment 45

The ACE2-Fc fusion protein of any one of Embodiments 1-44, wherein the ACE2 extracellular domain or fragment thereof comprises an amino acid sequence selected from the group consisting of SEQ ID NOs: 6, 8-38, and 51.

### Embodiment 46

The ACE2-Fc fusion protein of any one of Embodiments 1-44, wherein the ACE2-Fc fusion protein comprises an amino acid sequence selected from the group consisting of SEQ ID NOs: 52-59.

### Embodiment 47

The ACE2-Fc fusion protein of any one of Embodiments 1-44, wherein the ACE2-Fc fusion protein comprises an amino acid sequence of SEQ ID NO: 59.

### Embodiment 48

The ACE2-Fc fusion protein of any one of Embodiments 1-44, wherein the ACE2-Fc fusion protein comprises an amino acid sequence of SEQ ID NO: 50.

### Embodiment 49

The ACE2-Fc fusion protein of Embodiment 48, wherein the signal peptide of SEQ ID NO:2 is cleaved from the mature protein.

### Embodiment 50

The ACE2-Fc fusion protein of any one of Embodiments 1-49, wherein the ACE2-Fc fusion protein forms a homodimer.

### Embodiment 51

The ACE2-Fc fusion protein of any one of Embodiments 1-50, wherein the ACE2-Fc fusion protein binds to a coronavirus spike protein.

### Embodiment 52

The ACE2-Fc fusion protein of Embodiment 51, wherein the ACE2-Fc fusion protein binds to the coronavirus spike protein with Kd of about 1 nM to about 100 nM.

### Embodiment 53

The ACE2-Fc fusion protein of Embodiment 51 or 52, wherein the coronavirus is SARS-CoV-1 or SARS-CoV-2.

### Embodiment 54

The ACE2-Fc fusion protein of Embodiment 51 or 52, wherein the coronavirus is SARS-CoV-1 variant or SARS-CoV-2 variant.

### Embodiment 55

The ACE2-Fc fusion protein of any one of Embodiments 1-49, wherein the ACE2-Fc fusion protein binds and cleaves an ACE2 ligand.

### Embodiment 56

The ACE2-Fc fusion protein of Embodiment 55, wherein the ACE2 ligand is angiotensin I, angiotensin II, apelin, pro-dynorphin, or des-arg9-bradykinin.

### Embodiment 57

The ACE2-Fc fusion protein of Embodiment 18, wherein the one or more point mutations in the ACE2 extracellular domain or fragment thereof decreases the formation of higher-order multimers or aggregates.

### Embodiment 58

The ACE2-Fc fusion protein of Embodiment 18, wherein the one or more point mutations in the ACE2 extracellular domain or fragment thereof decreases binding to angiotensin II and/or decreases enzymatic activity when bound to angiotensin II.

### Embodiment 59

The ACE2-Fc fusion protein of Embodiment 18, wherein the one or more point mutations in the ACE2 extracellular domain or fragment thereof increases binding to viral spike protein.

### Embodiment 60

The ACE2-Fc fusion protein of any one of Embodiments 1-59, wherein the ACE2-Fc fusion protein demonstrates one or more of the following characteristics: (i) transport into the extracellular space via interaction with the FcRn; (ii) prolonged circulating half-life in human (e.g., greater than 24, 48, 72, or 96 hours); (iii) providing replacement ACE2 enzymatic activity in subjects with increased Angiotensin II; and (iv) reduced likelihood of Antibody Dependent Enhancement (ADE) compared to a fusion protein with an Fc domain that binds to low affinity Fc receptors.

### Embodiment 61

A recombinant polynucleotide encoding a monomer of the ACE2-Fc fusion protein of any one of Embodiments 1-60.

### Embodiment 62

The recombinant polynucleotide of Embodiment 61, further comprising a nucleic acid sequence encoding a signal peptide.

### Embodiment 63

An expression vector comprising the recombinant polynucleotide of Embodiment 61 or 62.

### Embodiment 64

A host cell comprising the expression vector of Embodiment 63.

### Embodiment 65

A method of treating or preventing one or more diseases or disorders, the method comprising administering the ACE2-Fc fusion protein of any one of Embodiments 1-60 to a subject in need thereof.

### Embodiment 66

The method of Embodiment 65, wherein the subject is human.

### Embodiment 67

The method of Embodiment 65 or 66, wherein the ACE2-Fc fusion protein is administered once per day, once per week, or multiple times per day or per week.

### Embodiment 68

The method of any one of Embodiments 65-67, wherein the ACE2-Fc fusion protein is administered at dose of about 0.001 mg/kg to about 1000 mg/kg of body weight per day.

### Embodiment 69

The method of any one of Embodiments 65-68, wherein the ACE2-Fc fusion protein is administered intravenously, subcutaneously, orally, intranasally, buccally, sublingually, intraperitoneally, or intramuscularly.

### Embodiment 69A

The method of any one of Embodiments 65-69, wherein the one or more diseases or disorders is caused by influenza.

### Embodiment 70

The method of Embodiment 65-69, wherein the one or more diseases or disorders is caused by a coronavirus.

### Embodiment 71

The method of Embodiment 70, wherein the coronavirus is SARS-CoV-1 or SARS-CoV-2.

### Embodiment 72

The method of Embodiment 71, wherein the coronavirus is a SARS-CoV-1 variant or a SARS-CoV-2 variant.

### Embodiment 73

The method of any one of Embodiments 65-69, wherein the one or more diseases or disorders is selected from the group consisting of cardiovascular disease, hypertension, cardiopulmonary disease, acute lung injury, acute respiratory distress syndrome, pulmonary fibrosis, diabetes-related micro- and macro-vascular diseases, metabolic syndrome, stress-related disorders, liver disease, kidney disease, ocular disorders, endometriosis, a neurodegenerative disease, an endocrine disorder, a granulomatous disease, a non-granulomatous disease, arthritis, cancer, sepsis, a mood or anxiety disorder, inflammation and autoimmunity.

### Embodiment 74

The method of Embodiment 65-73, wherein the ACE2-Fc fusion protein has an EC50 value of less than about 10 μM, less than about 1 μM, less than about 0.1 μM, less than about 0.01 μM, or less than about 0.001 μM when assaying binding of ACE2 to viral spike proteins.

### Embodiment 75

A composition comprising a plurality of ACE2-Fc fusion proteins of any one of Embodiments 1-60, wherein the composition comprises at least 80% homodimers w/w.

### Embodiment 76

The composition of Embodiment 75, comprising at least 85% w/w, at least 90% w/w at least 95% w/w at least 96% w/w, at least 97% w/w, at least 98% w/w, or at least 99% w/w homodimers.

### Embodiment 78

A method of treating a disease or disorder in a subject in need thereof comprising detecting a level of angiotensin II (Ang II) in the subject and administering the ACE2-Fc fusion protein of any one of claims 1-60 to the subject if an elevated level of Ang II is detected.

### Embodiment 79

A method of treating a disease or disorder in a subject in need thereof comprising detecting a level of des-arg-9-bradykinin in the subject and administering the ACE2-Fc fusion protein of any one of claims 1-60 to the subject if an elevated level of des-arg bradykinin is detected.

### Embodiment 80

A method of treating a disease or disorder in a subject in need thereof comprising detecting a level of Ang 1-7 in the subject and administering the ACE2-Fc fusion protein of any one of Embodiments 1-60 to the subject if a diminished level of Ang 1-7 is detected.

### Embodiment 81

A method of treating a disease or disorder in a subject in need thereof comprising detecting a ratio of Ang II to Ang 1-7 administering the ACE2-Fc fusion protein of any one of Embodiments 1-60 to the subject if an elevated level Ang II/Ang 1-7 ratio is detected.

### Embodiment 82

The method of any one of Embodiments 77-81, wherein the detected level of Ang II, elevated level of des-arg-9-bradykinin, or elevated ratio of Ang II to Ang 1-7 is elevated relative to the subject's historical level or ratio.

### Embodiment 83

The method of any one of Embodiments 77-81, wherein the detected level of Ang II, elevated level of des-arg-9-bradykinin, or elevated ratio of Ang II to Ang 1-7 is elevated relative to the level or ratio detected in a healthy control population.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. An angiotensin converting enzyme 2 (ACE2) Fc fusion protein comprising:
an ACE2 extracellular domain or fragment thereof; and
one or more Fc domains.

2. A homodimeric angiotensin converting enzyme 2 (ACE2) Fc fusion protein comprising a first and a second polypeptide monomer, wherein each monomer comprises
an ACE2 extracellular domain or fragment thereof; and
one or more Fc domain monomers,
wherein the one or more Fc domain monomers in the first polypeptide monomer associate with the one or more Fc domain monomers in the second polypeptide monomer to form one or more Fc domains.

3. The ACE2-Fc fusion protein of claim 1 or 2, wherein the one or more Fc domains demonstrate reduced binding to one or more low affinity Fcγ receptors compared to a wild type IgG1 Fc domain.

4. The ACE2-Fc fusion protein of any one of claims 1-3, wherein the one or more Fc domains are IgG4.

5. The ACE2-Fc fusion protein of any one of claims 1-3, wherein the one or more Fc domains are IgG1 or IgG3 Fc domains that have been mutated to reduce binding to one or more low affinity Fcγ receptors.

6. The ACE2-Fc fusion protein of any one of claims 1-5, wherein the ACE2 extracellular domain comprises an amino acid sequence that is at least 90%, 91%, 92%, 93%, 94%, or 95% identical to SEQ ID NO: 6.

7. An angiotensin converting enzyme 2 (ACE2) Fc fusion protein comprising:
an ACE2 extracellular domain fragment; and
one or more Fc domains, wherein the one or more Fc domains demonstrate reduced binding to one or more low affinity Fcγ receptors compared to a wild type IgG1 Fc domain.

8. A dimeric angiotensin converting enzyme 2 (ACE2) Fc fusion protein a first and a second polypeptide monomers, wherein each monomer comprises:
an ACE2 extracellular domain fragment; and
one or more Fc domain monomers,
wherein the one or more Fc domain monomers in the first polypeptide monomer associate with the one or more Fc domain monomers in the second polypeptide monomer to form one or more Fc domains, and
wherein the one or more Fc domains demonstrate reduced binding to one or more low affinity Fey receptors compared to a wild type IgG1 Fc domain.

9. The ACE2-Fc fusion protein of any one of claims 1-8, wherein the ACE2 extracellular domain fragment comprises an amino acid sequence that is at least 90%, 91%, 92%, 93%, 94%, or 95% identical to of SEQ ID NO: 8.

10. A dimeric angiotensin converting enzyme 2 (ACE2) Fc fusion protein comprising a first and a second polypeptide chain,
wherein the first polypeptide chain comprises an ACE2 extracellular domain or ligand-binding fragment thereof, and a first Fc domain monomer polypeptide chain; and
the second polypeptide chain comprises an Fc domain monomer polypeptide chain.

11. The ACE2-Fc fusion protein of claim 10, wherein the second polypeptide chain further comprises an ACE2 extracellular domain or ligand-binding fragment thereof.

12. The ACE2-Fc fusion protein of claim 10 or 11, wherein the first Fc domain monomer polypeptide chain and the second Fc domain monomer polypeptide chain of the second polypeptide chain form an Fc domain.

13. The ACE2-Fc fusion protein of embodiment 12, wherein the ACE2 Fc fusion protein is a homodimer.

14. The ACE2-Fc fusion protein of any one of claims 1-13, wherein the ACE2 extracellular domain is a ligand-binding fragment thereof.

15. The ACE2-Fc fusion protein of any one of claims 10-14, wherein the ACE2 extracellular domain fragment comprises an amino acid sequence that is at least 90%, 91%, 92%, 93%, 94%, or 95% identical to of SEQ ID NO: 8.

16. The ACE2-Fc fusion protein of any one of claims 1-15, wherein the ACE2 extracellular domain or fragment thereof further comprises a signal peptide of SEQ ID NO: 2.

17. The ACE2-Fc fusion protein of claim 15, wherein the signal peptide is cleaved from the mature protein.

18. The ACE2-Fc fusion protein of any one of claims 1-17, wherein the ACE2 extracellular domain or fragment thereof comprises one or more point mutations.

19. The ACE2-Fc fusion protein of claim 18, wherein the ACE2 extracellular domain or fragment thereof comprises a point mutation at position 82 of SEQ ID NO: 5 or SEQ ID NO: 7.

20. The ACE2-Fc fusion protein of claim 19, wherein the ACE2 extracellular domain or fragment thereof comprises the point mutation M82A, M82D, M82N, or M82S.

21. The ACE2-Fc fusion protein of claim 18, wherein the ACE2 extracellular domain or fragment thereof comprises a point mutation at position 30 of SEQ ID NO: 5 or SEQ ID NO: 7.

22. The ACE2-Fc fusion protein of claim 21, wherein the ACE2 extracellular domain or fragment thereof comprises the point mutation D30E or D30Q.

23. The ACE2-Fc fusion protein of claim 18, wherein the ACE2 extracellular domain or fragment thereof comprises a point mutation at position 31, 34, and/or 38 of SEQ ID NO: 5 or SEQ ID NO: 7.

24. The ACE2-Fc fusion protein of claim 23, wherein the ACE2 extracellular domain or fragment thereof comprises a K31T point mutation.

25. The ACE2-Fc fusion protein of claim 23, wherein the ACE2 extracellular domain or fragment thereof comprises a H34Q point mutation.

26. The ACE2-Fc fusion protein of claim 23, wherein the ACE2 extracellular domain or fragment thereof comprises a D38E point mutation.

27. The ACE2-Fc fusion protein of claim 18, wherein the ACE2 extracellular domain or fragment thereof comprises one or more point mutations selected from the group consisting of D30E, K31T, H34Q, and D38E.

28. The ACE2-Fc fusion protein of claim 18, wherein the ACE2 extracellular domain or fragment thereof comprises a point mutation at position 139 of SEQ ID NO: 5 or SEQ ID NO: 7.

29. The ACE2-Fc fusion protein of claim 28, wherein the ACE2 extracellular domain or fragment thereof comprises the point mutation Q139A, Q139S, or Q139V.

30. The ACE2-Fc fusion protein of claim 18, wherein the ACE2 extracellular domain or fragment thereof comprises a point mutation at position 175 of SEQ ID NO: 5 or SEQ ID NO: 7.

31. The ACE2-Fc fusion protein of claim 30, wherein the ACE2 extracellular domain or fragment thereof comprises the point mutation Q175A, Q175S, or Q175V.

32. The ACE2-Fc fusion protein of claim 18, wherein the ACE2 extracellular domain or fragment thereof comprises a point mutation at position 374 and/or position 378 of SEQ ID NO: 5 or SEQ ID NO: 7.

33. The ACE2-Fc fusion protein of claim 32, wherein the ACE2 extracellular domain or fragment thereof comprises the point mutation H374S, H374A, or H374V and/or H378S, H378A, or H378V.

34. The ACE2-Fc fusion protein of claim 18, wherein the ACE2 extracellular domain or fragment thereof comprises the point mutations M82N, Q139A, H374S, and H378S.

35. The ACE2-Fc fusion protein of any one of claim 1-3 or 5-34, wherein the Fc domain is an IgG1 Fc domain.

36. The ACE2-Fc fusion protein of claim 35, wherein the IgG1 Fc domain comprises an IgG1 hinge, an IgG1 CH2 domain, and an IgG1 CH3 domain.

37. The ACE2-Fc fusion protein of claim 36, wherein the IgG1 Fc domain comprises an amino acid sequence of SEQ ID NO: 39.

38. The ACE2-Fc fusion protein of any one of claim 1-4 or 6-34, wherein the Fc domain is an IgG4 Fc domain.

39. The ACE2-Fc fusion protein of claim 38, wherein the IgG4 Fc domain comprises an IgG4 hinge, an IgG4 CH2 domain, and an IgG4 CH3 domain.

40. The ACE2-Fc fusion protein of claim 39, wherein the IgG4 Fc domain comprises an amino acid sequence of SEQ ID NO: 42.

41. The ACE2-Fc fusion protein of any one of claims 1-40, further comprising a signal peptide, wherein the signal peptide comprises an amino acid sequence of SEQ ID NO: 2.

42. The ACE2-Fc fusion protein of claim 41, wherein the signal peptide is cleaved from the ACE2-Fc fusion protein.

43. The ACE2-Fc fusion protein of claim 42, wherein the signal peptide is cleaved between amino acid positions 17 and 18 of the ACE2-Fc fusion protein comprising the amino acid sequence selected from the group consisting of SEQ ID NO: 43-50.

44. The ACE2-Fc fusion protein of claim 42, wherein the signal peptide is cleaved between amino acid positions 19 and 20 of the ACE2-Fc fusion protein comprising the amino acid sequence selected from the group consisting of SEQ ID NO: 43-50.

45. The ACE2-Fc fusion protein of any one of claims 1-44, wherein the ACE2 extracellular domain or fragment thereof comprises an amino acid sequence selected from the group consisting of SEQ ID NOs: 6, 8-38, and 51.

46. The ACE2-Fc fusion protein of any one of claims 1-44, wherein the ACE2-Fc fusion protein comprises an amino acid sequence selected from the group consisting of SEQ ID NOs: 52-59.

47. The ACE2-Fc fusion protein of any one of claims 1-44, wherein the ACE2-Fc fusion protein comprises an amino acid sequence of SEQ ID NO: 59.

48. The ACE2-Fc fusion protein of any one of claims 1-44, wherein the ACE2-Fc fusion protein comprises an amino acid sequence of SEQ ID NO: 50.

49. The ACE2-Fc fusion protein of claim 48, wherein the signal peptide of SEQ ID NO:2 is cleaved from the mature protein.

50. The ACE2-Fc fusion protein of any one of claims 1-49, wherein the ACE2-Fc fusion protein forms a homodimer.

51. The ACE2-Fc fusion protein of any one of claims 1-50, wherein the ACE2-Fc fusion protein binds to a coronavirus spike protein.

52. The ACE2-Fc fusion protein of claim 51, wherein the ACE2-Fc fusion protein binds to the coronavirus spike protein with Kd of about 1 nM to about 100 nM.

53. The ACE2-Fc fusion protein of claim 51 or 52, wherein the coronavirus is SARS-CoV-1 or SARS-CoV-2.

54. The ACE2-Fc fusion protein of claim 51 or 52, wherein the coronavirus is SARS-CoV-1 variant or SARS-CoV-2 variant.

55. The ACE2-Fc fusion protein of any one of claims 1-49, wherein the ACE2-Fc fusion protein binds and cleaves an ACE2 ligand.

56. The ACE2-Fc fusion protein of claim 55, wherein the ACE2 ligand is angiotensin I, angiotensin II, apelin, pro-dynorphin, or des-arg⁹-bradykinin.

57. The ACE2-Fc fusion protein of claim 18, wherein the one or more point mutations in the ACE2 extracellular domain or fragment thereof decreases the formation of higher-order multimers or aggregates.

58. The ACE2-Fc fusion protein of claim 18, wherein the one or more point mutations in the ACE2 extracellular domain or fragment thereof decreases binding to angiotensin II and/or decreases enzymatic activity when bound to angiotensin II.

59. The ACE2-Fc fusion protein of claim 18, wherein the one or more point mutations in the ACE2 extracellular domain or fragment thereof increases binding to viral spike protein.

60. The ACE2-Fc fusion protein of any one of claims 1-59, wherein the ACE2-Fc fusion protein demonstrates one or more of the following characteristics:
(i) transport into the extracellular space via interaction with the FcRn;
(ii) prolonged circulating half-life in human (e.g., greater than 24, 48, 72, or 96 hours);
(iii) providing replacement ACE2 enzymatic activity in subjects with increased Angiotensin II; and
(iv) reduced likelihood of Antibody Dependent Enhancement (ADE) compared to a fusion protein with an Fc domain that binds to low affinity Fc receptors.

61. A recombinant polynucleotide encoding a monomer of the ACE2-Fc fusion protein of any one of claims 1-60.

62. The recombinant polynucleotide of claim 61, further comprising a nucleic acid sequence encoding a signal peptide.

63. An expression vector comprising the recombinant polynucleotide of claim 61 or 62.

64. A host cell comprising the expression vector of claim 63.

65. A method of treating or preventing one or more diseases or disorders, the method comprising administering the ACE2-Fc fusion protein of any one of claims 1-60 to a subject in need thereof.

66. The method of claim 65, wherein the subject is human.

67. The method of claim 65 or 66, wherein the ACE2-Fc fusion protein is administered once per day, once per week, or multiple times per day or per week.

68. The method of any one of claims 65-67, wherein the ACE2-Fc fusion protein is administered at dose of about 0.001 mg/kg to about 1000 mg/kg of body weight per day.

69. The method of any one of claims 65-68, wherein the ACE2-Fc fusion protein is administered intravenously, subcutaneously, orally, intranasally, buccally, sublingually, intraperitoneally, or intramuscularly.

70. The method of claim 65-69, wherein the one or more diseases or disorders is caused by influenza.

71. The method of claim 65-69, wherein the one or more diseases or disorders is caused by a coronavirus.

72. The method of claim 70, wherein the coronavirus is SARS-CoV-1 or SARS-CoV-2.

73. The method of claim 71, wherein the coronavirus is a SARS-CoV-1 variant or a SARS-CoV-2 variant.

74. The method of any one of claims 65-69, wherein the one or more diseases or disorders is selected from the group consisting of cardiovascular disease, hypertension, cardiopulmonary disease, acute lung injury, acute respiratory distress syndrome, acute lung injury, pulmonary fibrosis, diabetes-related micro- and macro-vascular diseases, metabolic syndrome, stress-related disorders, liver disease, kidney disease, ocular disorders, endometriosis, a neurodegenerative disease, an endocrine disorder, a granulomatous disease including sarcoidosis, a non-granulomatous disease, arthritis, cancer, sepsis, a mood or anxiety disorder, inflammation, inflammatory pain, and autoimmunity.

75. The method of claim 65-73, wherein the ACE2-Fc fusion protein has an EC50 value of less than about 10 μM, less than about 1 μM, less than about 0.1 μM, less than about 0.01 μM, or less than about 0.001 μM when assaying binding of ACE2 to viral spike proteins.

76. A composition comprising a plurality of ACE2-Fc fusion proteins of any one of claims 1-60, wherein the composition comprises at least 80% homodimers w/w.

77. The composition of claim 75, comprising at least 85% w/w, at least 90% w/w at least 95% w/w at least 96% w/w, at least 97% w/w, at least 98% w/w, or at least 99% w/w homodimers.

78. A method of treating a disease or disorder in a subject in need thereof comprising detecting a level of angiotensin II (Ang II) in the subject and administering the ACE2-Fc fusion protein of any one of claims 1-60 to the subject if an elevated level of Ang II is detected.

79. A method of treating a disease or disorder in a subject in need thereof comprising detecting a level of des-arg-9-bradykinin in the subject and administering the ACE2-Fc fusion protein of any one of claims 1-60 to the subject if an elevated level of des-arg-9-bradykinin is detected.

80. A method of treating a disease or disorder in a subject in need thereof comprising detecting a level of Ang 1-7 in the subject and administering the ACE2-Fc fusion protein of any one of claims 1-60 to the subject if a diminished level of Ang 1-7 is detected.

81. A method of treating a disease or disorder in a subject in need thereof comprising detecting a ratio of Ang II to Ang 1-7 administering the ACE2-Fc fusion protein of any one of claims 1-60 to the subject if an elevated level Ang II/Ang 1-7 ratio is detected.

82. The method of any one of claims 77-81, wherein the detected level of Ang II, elevated level of des-arg-9-bradykinin, or elevated ratio of Ang II to Ang 1-7 is elevated relative to the subject's historical level or ratio.

83. The method of any one of claims 77-81, wherein the detected level of Ang II, elevated level of des-arg-9-bradykinin, or elevated ratio of Ang II to Ang 1-7 is elevated relative to the level or ratio detected in a healthy control population.


SEQUENCE LISTING (65 sequences)

SEQ ID NO: 1 - protein, 20 aa, Artificial Sequence
  IgG kappa SP
METDTLLLWVLLLWVPGSTG

SEQ ID NO: 2 - protein, 17 aa, Artificial Sequence
  ACE2 SP
MSSSSWLLLSLVAVTAA

SEQ ID NO: 3 - protein, 805 aa, Artificial Sequence [sequence omitted]

SEQ ID NO: 4 - protein, 555 aa, Artificial Sequence
  HuACE2, full length, isoform 2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLL

SEQ ID NO: 5 - protein, 740 aa, Artificial Sequence
  HuACE2-SP-ECD (native SP)
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 6 - protein, 723 aa, Artificial Sequence
  HuACE2-ECD
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 7 - protein, 615 aa, Artificial Sequence
  HuACE2-SP-ECD615 (native SP)
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 8 - protein, 598 aa, Artificial Sequence
  HuACE2-ECD615
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 9 - protein, 598 aa, Artificial Sequence
  HuACE2-ECD615 (M82N)
QSTIEEQAKTFLDKFNHEAEDLFYQSSLASWNYNTNITEENVQNMNNAGDKWSAFLKEQSTLAQNYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 10 - protein, 598 aa, Artificial Sequence
  HuACE2-ECD615 (M82A)
QSTIEEQAKTFLDKFNHEAEDLFYQSSLASWNYNTNITEENVQNMNNAGDKWSAFLKEQSTLAQAYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 11 - protein, 598 aa, Artificial Sequence
  HuACE2-ECD615 (M82D)
QSTIEEQAKTFLDKFNHEAEDLFYQSSLASWNYNTNITEENVQNMNNAGDKWSAFLKEQSTLAQDYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 12 - protein, 598 aa, Artificial Sequence
  HuACE2-ECD615 (M82S)
QSTIEEQAKTFLDKFNHEAEDLFYQSSLASWNYNTNITEENVQNMNNAGDKWSAFLKEQSTLAQSYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 13 - protein, 598 aa, Artificial Sequence
  HuACE2-ECD615 (D30E)
QSTIEEQAKTFLEKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 14 - protein, 598 aa, Artificial Sequence
  HuACE2-ECD615 (K31T)
QSTIEEQAKTFLDTFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 15 - protein, 598 aa, Artificial Sequence
  HuACE2-ECD615 (H34Q)
QSTIEEQAKTFLDKFNQEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 16 - protein, 598 aa, Artificial Sequence
  HuACE2-ECD615 (D38E)
QSTIEEQAKTFLDKFNHEAEELFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 17 - protein, 598 aa, Artificial Sequence
  HuACE2-ECD615 (Q139S)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPSECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 18 - protein, 598 aa, Artificial Sequence
  HuACE2-ECD615 (Q139A)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPAECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 19 - protein, 598 aa, Artificial Sequence
  HuACE2-ECD615 (Q139V)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPVECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 20 - protein, 598 aa, Artificial Sequence
  HuACE2-ECD615 (Q175S)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKSLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 21 - protein, 598 aa, Artificial Sequence
  HuACE2-ECD615 (Q175A)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 22 - protein, 598 aa, Artificial Sequence
  HuACE2-ECD615 (Q175V)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKVLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 23 - protein, 598 aa, Artificial Sequence
  HuACE2-ECD615 (H374S)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHSEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 24 - protein, 598 aa, Artificial Sequence
  HuACE2-ECD615 (H374A)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHAEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 25 - protein, 598 aa, Artificial Sequence
  HuACE2-ECD615 (H374V)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHVEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 26 - protein, 598 aa, Artificial Sequence
  HuACE2-ECD615 (H378S)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGSIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 27 - protein, 598 aa, Artificial Sequence
  HuACE2-ECD615 (H378A)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGAIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 28 - protein, 598 aa, Artificial Sequence
  HuACE2-ECD615 (H378V)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGVIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 29 - protein, 598 aa, Artificial Sequence
  HuACE2-ECD615 (M82N/D38E)
QSTIEEQAKTFLDKFNHEAEELFYQSSLASWNYNTNITEENVQNMNNAGDKWSAFLKEQSTLAQNYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 30 - protein, 598 aa, Artificial Sequence
  HuACE2-ECD615 (M82A/D38E)
QSTIEEQAKTFLDKFNHEAEELFYQSSLASWNYNTNITEENVQNMNNAGDKWSAFLKEQSTLAQAYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 31 - protein, 598 aa, Artificial Sequence
  HuACE2-ECD615 (M82T/D30E)
QSTIEEQAKTFLEKFNHEAEDLFYQSSLASWNYNTNITEENVQNMNNAGDKWSAFLKEQSTLAQTYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 32 - protein, 598 aa, Artificial Sequence
  HuACE2-ECD615 (M82N/Q139A)
QSTIEEQAKTFLDKFNHEAEDLFYQSSLASWNYNTNITEENVQNMNNAGDKWSAFLKEQSTLAQNYPLQEIQNLTVKLQLQALQQNGSSVLSEDKSKRLNTILNTMSTIYSTGKVCNPDNPAECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 33 - protein, 598 aa, Artificial Sequence
  HuACE2-ECD615 (M82N/Q175A)
QSTIEEQAKTFLDKFNHEAEDLFYQSSLASWNYNTNITEENVQNMNNAGDKWSAFLKEQSTLAQNYPLQEIQNLTVKLQLQALQQNGSSVLSEDKSKRLNTILNTMSTIYSTGKVCNPDNPQECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 34 - protein, 598 aa, Artificial Sequence
  HuACE2-ECD615 (M82N/H374S)
QSTIEEQAKTFLDKFNHEAEDLFYQSSLASWNYNTNITEENVQNMNNAGDKWSAFLKEQSTLAQNYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHSEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 35 - protein, 598 aa, Artificial Sequence
  HuACE2-ECD615 (M82N/H378S)
QSTIEEQAKTFLDKFNHEAEDLFYQSSLASWNYNTNITEENVQNMNNAGDKWSAFLKEQSTLAQNYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGSIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 36 - protein, 598 aa, Artificial Sequence
  HuACE2-ECD615 (D30Q/E35K)
QSTIEEQAKTFLQKFNHKAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 37 - protein, 598 aa, Artificial Sequence
  HuACE2-ECD615 (M82N/ Q175A/H374S/H378S)
QSTIEEQAKTFLDKFNHEAEDLFYQSSLASWNYNTNITEENVQNMNNAGDKWSAFLKEQSTLAQNYPLQEIQNLTVKLQLQALQQNGSSVLSEDKSKRLNTILNTMSTIYSTGKVCNPDNPQECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHSEMGSIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 38 - protein, 598 aa, Artificial Sequence
  HuACE2-ECD615 (M82N/Q139A/H374S/H378S)
QSTIEEQAKTFLDKFNHEAEDLFYQSSLASWNYNTNITEENVQNMNNAGDKWSAFLKEQSTLAQNYPLQEIQNLTVKLQLQALQQNGSSVLSEDKSKRLNTILNTMSTIYSTGKVCNPDNPAECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHSEMGSIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 39 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 41 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 42 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 975 aa, Artificial Sequence
  ACE2(ECD)-IgG1
METDTLLLWVLLLWVPGSTGQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 971 aa, Artificial Sequence
  ACE2(ECD)-IgG2
METDTLLLWVLLLWVPGSTGQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 45 - protein, 1022 aa, Artificial Sequence
  ACE2(ECD)-IgG3
METDTLLLWVLLLWVPGSTGQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 46 - protein, 972 aa, Artificial Sequence
  ACE2(ECD)-IgG4
METDTLLLWVLLLWVPGSTGQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 850 aa, Artificial Sequence
  ACE2(ECD615)-IgG1
METDTLLLWVLLLWVPGSTGQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 846 aa, Artificial Sequence
  ACE2(ECD615)-IgG2
METDTLLLWVLLLWVPGSTGQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 49 - protein, 897 aa, Artificial Sequence
  ACE2(ECD615)-IgG3
METDTLLLWVLLLWVPGSTGQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 50 - protein, 847 aa, Artificial Sequence
  ACE2(ECD615)-IgG4
METDTLLLWVLLLWVPGSTGQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 598 aa, Artificial Sequence
  HuACE2-ECD615 (Y41H/Q42E)
QSTIEEQAKTFLDKFNHEAEDLFHESSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 52 - protein, 955 aa, Artificial Sequence
  ACE2(ECD)-IgG1, No signal peptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 951 aa, Artificial Sequence
  ACE2(ECD)-IgG2, No signal peptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 54 - protein, 1002 aa, Artificial Sequence
  ACE2(ECD)-IgG3, No signal peptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 55 - protein, 952 aa, Artificial Sequence
  ACE2(ECD)-IgG4, No signal peptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 830 aa, Artificial Sequence
  ACE2(ECD615)-IgG1, No signal peptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 826 aa, Artificial Sequence
  ACE2(ECD615)-IgG2, No signal peptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 58 - protein, 877 aa, Artificial Sequence
  ACE2(ECD615)-IgG3, No signal peptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 59 - protein, 827 aa, Artificial Sequence
  ACE2(ECD615)-IgG4, No signal peptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 1258 aa, Artificial Sequence
  synthetic coronavirus spike protein
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 61 - protein, 1195 aa, Artificial Sequence
  synthetic coronavirus spike protein
VNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRAAASVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 62 - protein, 1195 aa, Artificial Sequence
  synthetic coronavirus spike protein
VNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRAAASVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 63 - protein, 1198 aa, Artificial Sequence
  synthetic coronavirus spike protein
VNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRAAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWP

SEQ ID NO: 64 - protein, 1198 aa, Artificial Sequence
  synthetic coronavirus spike protein
VNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRAAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 65 - protein, 1198 aa, Artificial Sequence; identical to SEQ ID NO: 64
  synthetic coronavirus spike protein